(19) United States
(12) Patent Application Publication
(10) Pub. No.: US 2023/0293555 A1
(43) Pub. Date: Sep. 21, 2023

(54) COMPOSITIONS COMPRISING CANNABIDIOL AND FLAVANONES

(71) Applicant: YUZU LLC, Las Vegas, NV (US)

(72) Inventors: Nicola Michael SPIRTOS, Las Vegas, NV (US); Chad BREY, Bakersfield, CA (US)

(21) Appl. No.: 18/009,757

(22) PCT Filed: Jul. 1, 2021
(86) PCT No.: PCT/US2021/040115

§ 371 Date: Dec. 12, 2022

Related U.S. Application Data
(60) Provisional application No. 63/055,458, filed on Jul. 23, 2020.
(60) Provisional application No. 63/047,550, filed on Jul. 2, 2020.

(51) Int. Cl. A61K 31/00; A23L 33/105; A23L 33/125; A61K 31/352; A61K 47/36; A61K 31/375

(52) U.S. Cl. CPC A61K 31/658; A23L 33/105; A23L 33/125; A61K 31/352; A61K 47/36; A61K 31/375

(57) ABSTRACT

Disclosed here are compositions comprising a cannabidiol (CBD), a flavanone and one or more additional compounds and uses thereof. A composition described herein may be formulated as a dietary supplement, a pharmaceutical formulation, or a cosmetic. In some cases, the composition described herein can be used to treat arthritis, such as acute arthritis or chronic arthritis. The composition described herein can be administered to a human or an animal such as a pet.

### CROSS REFERENCE TO RELATED APPLICATIONS

This application claims the benefit of U.S. Provisional Application Ser. No. 63/047,550 filed Jul. 2, 2020, and U.S. Provisional Application Ser. No. 63/055,458 filed Jul. 23, 2020 the disclosures of which are incorporated herein by reference in their entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein are compositions. In some embodiments, a composition can comprise a cannabidiol (CBD), a flavanone or a salt thereof, and one or more of: a glucosamine, a salt thereof, a glycosaminoglycan, a salt thereof, a vitamin, a derivative thereof, a salt thereof, or methylsulfonylmethane (MSM). In some embodiments, a composition can be in the form of a capsule, a tablet, a gummy, an oil, a tincture, a lotion, a cream, a balm, a candy, a chocolate, a food, a drink, a liquid, an oil, a syrup or any combination thereof. In some embodiments, a composition can further comprise a monoterpene, a derivative thereof, a salt thereof or any combination thereof. In some embodiments, a composition can further comprise: a mineral, a salt thereof, or a chelated form thereof, an amino acid or a salt thereof, at least a portion of a plant or an extract thereof, an oil, a fatty acid, or any combination thereof. In some embodiments, a composition can further comprise: a sugar, a sucralose, an aspartame, a stevia, or any combination thereof. In some embodiments, a composition can comprise substantially no Δ9-tetrahydrocannabinol (THC). In some embodiments, the composition can further comprise Δ8-THC. In some embodiments, a composition can be a dietary supplement and can be in the form of a capsule, a gummy, a candy, a chocolate, a beverage, a food, a liquid, an oil, a syrup or any combination thereof; or can be a medical food and can be in the form of a capsule, a gummy, a candy, a chocolate, a beverage, a food, a liquid, an oil, a syrup or any combination thereof. In some embodiments, a CBD can comprise cannabidiol, cannabidiolic acid, or any combination thereof. In some embodiments, a CBD can be formulated as a powder, an oil, a liquid, or any combination thereof. In some embodiments, a CBD can be at least partially water soluble. In some embodiments, a composition can comprise a flavanone or salt thereof and the flavanone or salt thereof can comprise naringenin, a salt thereof, a derivative of naringenin, a salt thereof, naringin, a salt thereof, a derivative of naringin, a salt thereof, or any combination thereof. In some embodiments, a flavanone can be in a form of an extract from: a grapefruit, a sour orange, a tart cherry, a citrus fruit, a tomato or any combination thereof. In some embodiments, a composition can comprise the salt of glucosamine and the salt of glucosamine can comprise glucosamine sulfate. In some embodiments, a composition can comprise a salt of glycosaminoglycan and the salt of glycosaminoglycan can comprise chondroitin sulfate, dermatan sulfate, or any combination thereof. In some embodiments, a composition can comprise a glycosaminoglycan or salt thereof and the glycosaminoglycan or salt thereof can comprise hyaluronan or a salt thereof. In some embodiments, a composition can comprise a salt of glycosaminoglycan and the salt of glycosaminoglycan can comprise keratan sulfate. In some embodiments, a composition can comprise a vitamin, a derivative thereof, or a salt thereof and the vitamin can comprise: vitamin A, vitamin C, vitamin D, vitamin E, vitamin K, vitamin B1, vitamin B2, vitamin B3, vitamin B5, vitamin B6, vitamin B7, vitamin B9, vitamin B12, a derivative of any of these, a salt of any of these, or any combination thereof. In some embodiments, a composition can comprise a vitamin, a derivative thereof, or a salt thereof and the vitamin can comprise a vitamin C or a salt thereof. In some embodiments, a vitamin, a derivative thereof, or a salt thereof can comprise a salt of vitamin C and the salt of vitamin C can comprise calcium ascorbate. In some embodiments, a composition can comprise a monoterpene or salt thereof and the monoterpene can comprise D-limonene or a salt thereof. In some embodiments, a composition can comprise a mineral, a chelated form thereof, a salt thereof, or any combination thereof and the mineral can comprise: a calcium, a phosphorus, a potassium, a sulfur, a sodium, a chloride, a magnesium, an iron, a zinc, a copper, a manganese, an iodine, a selenium, a molybdenum, a chromium, a fluoride, a chelated form thereof, a salt thereof, or any combination thereof. In some embodiments, a composition can comprise an at least the portion of a plant or an extract thereof and the at least a portion of a plant or the extract thereof can comprise: Boswellia serrata, lemon grass, eucalyptus, bromelain, capsaicin, cat's claw, curcumin, evening primrose, ginger, ginkgo, green tea, flaxseed, pine bar, rose hips, St. John's wort, Devil's claw, hemp, stinging nettle, thunder god vine, valerian root, any portion thereof, any extract thereof, a tincture thereof, or any combination thereof. In some embodiments, a composition can comprise an oil and the oil can comprise: fish oil, avocado oil, soybean oil, black currant oil, lemon grass oil, eucalyptus oil, frankincense oil, medium-chain triglyceride (MCT) oil, hemp oil, borage oil or any combination thereof. In some embodiments, a composition can comprise a fatty acid and the fatty acid can comprise a short chain fatty acid, a medium chain fatty acid, a long chain fatty acid, an ester of any of these, a salt of any of these, or any combination thereof. In some embodiments, a composition can comprise a fatty acid and the fatty acid can comprise a saturated fatty acid, an unsaturated fatty acid, a monounsaturated fatty acid, a polyunsaturated fatty acid, an ester of any of these, a salt of any of these or any combination thereof. In some embodiments, a fatty acid can comprise a saturated fatty acid, an unsaturated fatty acid, a monounsaturated fatty acid, a polyunsaturated fatty acid, or any combination thereof and the saturated fatty acid, the unsaturated fatty acid, the monounsaturated fatty acid, the polyunsaturated fatty acid, or any combination thereof can comprise arachidonic acid, linoleic acid, palmitic acid, oleic acid, adrenic acid, docosapentaenoic acid, mead acid, an ester of any of these, a salt of any of these or any combination thereof. In some embodiments, a composition can comprise a fatty acid and the fatty acid can comprise an omega-3 fatty acid, an omega-6 fatty acid, an omega-9 fatty acid, an ester of any of these, a salt of any of these or any combination thereof. In some embodiments, a fatty acid can comprise an omega-3 fatty acid and the omega-3 fatty acid can comprise eicosapentaenoic acid (EPA), docosahexaenoic acid (DHA), alpha-linolenic acid (ALA), an ester of any of these, a salt of any of these or any combination thereof. In some embodiments, a capsule can be a hydroxymethylcellulose capsule. In some embodiments, a candy can be in the form of: a caramel, a fruit candy drop, or any combination thereof. In some embodiments, a chocolate can be in the form of: a blueberry chocolate, a cranberry chocolate, a fruit flavored chocolate, a citrus flavored chocolate, a fruit covered chocolate, a chocolate bar, a chocolate caramel, a coffee bean chocolate or any combination thereof. In some embodiments, a balm can further comprise shea butter, cocoa butter, bees wax or any combination thereof. In some embodiments, a cream can further comprise hemp cream. In some embodiments, a gummy can further comprise carrageenan.

Also disclosed herein are methods comprising administering a composition to a subject in unit dose form. In some embodiments, administering can be by oral ingestion, topical application, or inhalation. In some embodiments, administering can comprise oral ingestion and the oral ingestion can comprise oral ingestion of a food, a liquid, a gel, a capsule, or any combination thereof. In some embodiments, administering can comprise topical application and the topical application can comprise topical application of a lotion, a tincture, a balm, a cream, an oil, a gel, a butter, a liquid, a spray, an ointment, a paste, a jelly, or any combination thereof. In some embodiments, administering can comprise inhalation and the inhalation can comprise inhalation by a diffuser. In some embodiments, administering can be performed at least about: 1 time per day, 2 times per day, 3 times per day, 4 times per day, 5 times per day, or 6 times per day. In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or about 50 years to about 130 years. In some embodiments, a subject can have joint pain, joint inflammation, joint immobilization, joint deterioration or any combination thereof. In some embodiments, a subject may have been previously diagnosed with acute arthritis, chronic arthritis or any combination thereof. In some embodiments, a subject may have been previously diagnosed with acute arthritis and the acute arthritis can comprise: an infection, gout, calcium pyrophosphate arthritis, bursitis, tendinitis or any combination thereof. In some embodiments, a subject may have been previously diagnosed with chronic arthritis and the chronic arthritis can comprise: rheumatoid arthritis, psoriatic arthritis, lupus, erythematosus, osteoarthritis, juvenile idiopathic arthritis, fibromyalgia, polymyalgia rheumatica or any combination thereof. In some embodiments, a subject can be a human. In some embodiments, a human can be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old. In some embodiments, a subject can be an animal. In some embodiments, an animal can comprise a pet. In some embodiments, an animal can comprise a pet and the pet can comprise a dog, a cat, a rodent, a horse, a goat, or a rabbit. In some embodiments, an animal can be about 1 day to about 10 months old, from about 9 months to about 24 months old, from about 1 year to about 8 years old, from about 5 years to about 25 years old, or from about 20 years to about 50 years old.

Also disclosed herein are methods of treating or preventing a disease comprising administering a composition to a subject as a pharmaceutical composition in unit dose form. In some embodiments, the disease can comprise treating or preventing acute arthritis, chronic arthritis or any combination thereof. In some embodiments, a disease can comprise acute arthritis and the acute arthritis can comprise: an infection, gout, calcium pyrophosphate arthritis, bursitis, tendinitis or any combination thereof. In some embodiments, a disease can comprise chronic arthritis and the chronic arthritis can comprise: rheumatoid arthritis, psoriatic arthritis, lupus, erythematosus, osteoarthritis, juvenile idiopathic arthritis, fibromyalgia, polymyalgia rheumatica or any combination thereof. In some embodiments, prior to treating, a subject may have been diagnosed with acute arthritis, chronic arthritis, or any combination thereof. In some embodiments, a method can further comprise diagnosing a subject as having acute arthritis, chronic arthritis, or any combination thereof. In some embodiments, a diagnosis can comprise a physical examination, a radiological image, a blood test, an antibody test, or any combination thereof. In some embodiments, a diagnosis can comprise a radiological image and the radiological image can comprise: a computed tomography (CT) image, an X-Ray image, a magnetic resonance image (MRI), an ultrasound image, or any combination thereof. In some embodiments, a method can further comprise administering a second therapy to the subject. In some embodiments, a second therapy can comprise acetaminophen, an opioid, a nonsteroidal anti-inflammatory drug, methotrexate, hydroxychloroquine, prednisone, cortisone, a biological response modifier, a salt thereof, or any combination thereof. In some embodiments, a second therapy can comprise a biological response modifier and the biological response modifier can comprise: abatacept, adalimumab, adalimumab-atto, anakinra, certolizumab pegol, etanercept, etanercept-szzs, golimumab, infliximab, infliximab-dyyb, rituximab, sarilumab, tocilizumab, a biologically active fragment of any of these, a salt of any of these, or any combination thereof. In some embodiments, the second therapy can comprise a nonsteroidal anti-inflammatory drug and the nonsteroidal anti-inflammatory drug can comprise naproxen, ibuprofen, a salt of any of these, or any combination thereof. In some embodiments, a composition can comprise an excipient, a diluent, a carrier, or any combination thereof. In some embodiments, administering can be by oral ingestion, topical application, or inhalation. In some embodiments, administering can comprise oral ingestion and the oral ingestion can comprise oral ingestion of a food, a liquid, a gel, a capsule, a tablet, or any combination thereof. In some embodiments, administering can be by topical application and the topical application can comprise topical application of a lotion, a tincture, a balm, a cream, an oil, a gel, a butter, a liquid, a spray, an ointment, a paste, a jelly, or any combination thereof. In some embodiments, administering can comprise inhalation and the inhalation can comprise inhalation by a diffuser, an inhaler, a nebulizer or any combination thereof. In some embodiments, administering can be performed at least about: 1 time per day, 2 times per day, 3 times per day, 4 times per day, 5 times per day, or 6 times per day. In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or about 50 years to about 130 years. In some embodiments, a subject can be a subject in need thereof. In some embodiments, a subject can be a human. In some embodiments, a human can be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old. In some embodiments, a subject can be an animal. In some embodiments, an animal can comprise a pet. In some embodiments, an animal can comprise a pet and the pet can comprise a dog, a cat, a rodent, a horse, a goat, or a rabbit. In some embodiments, an animal can be about 1 day to about 10 months old, from about 9 months to about 24 months old, from about 1 year to about 8 years old, from about 5 years to about 25 years old, or from about 20 years to about 50 years old.

Also disclosed herein are methods of making compositions. In some embodiments, a method can comprise formulating the following into a composition: CBD, a flavanone or a salt thereof; and one or more of: a glucosamine, a salt thereof, a glycosaminoglycan, a salt thereof, a vitamin, a derivative thereof, a salt thereof, or methylsulfonylmethane (MSM).

Also disclosed herein are methods of making compositions. In some embodiments, a method can comprise formulating the following into a composition: CBD, naringenin or a salt thereof, and one or more of eucalyptus oil or lemon grass oil. In some embodiments, a composition can further comprise formulating D-limonene, a salt thereof, vitamin C, a salt thereof, MSM, a hemp cream, or any combination thereof into the composition. In some embodiments, a CBD can be at least partially water soluble. In some embodiments, a composition can be formulated as a cream or a lotion.

Also disclosed herein are methods of making compositions. In some embodiments, a method can comprise; formulating the following into a composition: a medium-chain triglyceride (MCT) oil, CBD, and naringenin or a salt thereof. In some embodiments, a composition can further comprise formulating shea butter, cocoa butter, bees wax, lemon grass oil, frankincense oil, eucalyptus oil, D-limonene or a salt thereof, MSM, vitamin C or a salt thereof or any combination thereof into a composition. In some embodiments, a composition can further comprise a flavor or a fragrance. In some embodiments, a CBD can be at least partially water soluble. In some embodiments, a composition can be formulated as a balm or a tincture.

Also disclosed herein are kits comprising a composition and a container.

### DETAILED DESCRIPTION

The methods and compositions disclosed herein can be used to supplement the diet, maintain health, or both in a subject. Also, the methods and compositions disclosed herein can be used for treating a disease or condition. In some cases, the methods and compositions disclosed herein can be applied as a cosmetic.

Unless otherwise indicated, open terms for example “contain,” “containing,” “include,” “including,” and the like mean comprising.

The singular forms “a”, “an”, and “the” can be used herein to include plural references unless the context clearly dictates otherwise. Accordingly, unless the contrary is indicated, the numerical parameters set forth in this application can be approximations that can vary depending upon the desired properties sought to be obtained.

When used herein, a percentage of a compound (e.g. THC) of a composition can be with respect to a total weight or a total volume of a composition. In some cases, a percentage of a component of a composition can be with respect to a total weight or a total volume of a composition.

A derivative of a compound disclosed herein, can refer to a chemical substance related structurally a compound disclosed herein. A derivative can be made from the structurally-related parent compound in one or more steps. The general physical and chemical properties of a derivative can be similar to a parent compound. In some embodiments, a compound disclosed herein can be an ester (e.g. a fatty acid). In some cases, an ester can be derived from a carboxylic acid. A carboxylic acid can contain the —COOH group, and in an ester the hydrogen in this group can be replaced by a hydrocarbon group. The hydrocarbon group can be an alkyl group (e.g. methyl or ethyl, or one comprising a benzene ring.) In some cases, an ester can refer to a chemical moiety with formula —COOR, where R can represent an alkyl, a cycloalkyl, an aryl, a heteroaryl, or a heteroalicyclic. In some instances, an ester can be an alkoxycarbonyl group appended to the parent molecule on an available carbon atom. In some cases, an ester can be an alkoxycarbonyl group appended to the parent molecule on one or more available aryl groups, cycloalkyl groups, heterocycle groups, or any combination thereof. In some cases, a compound containing an ester can be a compound considered to be prodrug. In some embodiments, an ester group can comprise, pivaloyloxymethyl, acetoxymethyl, phthalidyl, indanyl and methoxymethyl, or any combination thereof.

The term “subject,” “host,” “individual,” and “patient” can be used interchangeably herein to refer to animals, for example, mammalian animals. Any suitable mammal can be administered a composition as described herein or treated by a method as described herein. In some cases, a subject can be a pet. A pet can be mammal, a bird, a reptile, an amphibian or any animal. For example, a pet can be a dog, a cat, a mouse, a horse, a snake, a goat, a rabbit or any animal. Non-limiting examples of mammals can include humans, non-human primates (e.g., an ape, a gibbon, a chimpanzee, an orangutan, a monkey), domestic animals (e.g., a dog, a cat, a rabbit, and a rodent), farm animals (e.g., a horse, a cow, a goat, a sheep, a pig) and experimental animals (e.g., a mouse, a rat, a rabbit, a guinea pig). A bird can comprise a parakeet, a parrot, a love bird, or any bird. A reptile can comprise a snake, a turtle, a lizard, an alligator, or any combination thereof. Mammals or animals can be any age or at any stage of development, for example a mammal can be neonatal, infant, adolescent, adult or in utero. In some embodiments a mammal can be a human. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 year to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age. In some cases, an animal can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 year to about 100 years old. In some cases, an animal can be more than about: 1, 2, 5, 10, 20, 30, 40, 50, 60, 70, 80, 90, 100, 110, or 120 years of age. In some cases, an animal can be less than about: 1, 2, 5, 10, 20, 30, 40, 50, 60, 70, 80, 90, 100, 110, 120 or 130 years of age. An animal can be a male or a female. In some embodiments a subject can be a human. In some cases, a human can be a male. In some instances, a human can be a female. In some embodiments, a subject may be suspected of having a disease or condition. The subject can be a patient, such as a patient being treated for a condition or a disease, such as an arthritic disease (e.g. acute arthritis or chronic arthritis), a cancer, or a neurological condition. The subject may be predisposed to a risk of developing a condition or a disease such as chronic arthritis. The subject may be in remission from a condition or a disease, such as a cancer patient or an arthritis patient. The subject may be healthy or appear to be healthy. In some instances, the subject may have a comorbidity.

As used herein, the term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges, subranges, or both, of values are provided, the ranges or subranges can include the endpoints of the ranges or subranges. The terms “substantially”, “substantially no”, “substantially free”, and “approximately” may be used when describing a magnitude, a position or both to indicate that the value described can be within a reasonable expected range of values. For example, a numeric value may have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

An “effective amount” can be an amount of a therapeutic agent sufficient to achieve an intended purpose. An effective amount of a composition to treat or ameliorate a disease (e.g. chronic or acute arthritis) can be an amount of the composition sufficient to reduce or remove the symptoms of the disorder.

The terms “treating,” “treatment,” and the like can be used herein to mean obtaining a desired pharmacologic effect, physiologic effect, or any combination thereof. In some instances, an individual suffering from acute arthritis or chronic arthritis can be treated prophylactically with composition described herein and such prophylactic treatment can completely or partially prevent acute arthritis or chronic arthritis. In some instances, the composition herein can be used as a regenerative medicine. In some instances, the composition herein can be used for tissue repair to treat various conditions rising from injury or damage of a joint, a tissue, an organ, or any combination thereof. In some instances, the composition herein can be used to treat a chronic disease such as chronic arthritis. In some instances, an individual can be treated therapeutically (e.g., when an individual can be suffering from chronic arthritis or acute arthritis), such therapeutic treatment can cause a partial or a complete cure for the disease or disorder. In some instances, a treatment can reverse an adverse effect attributable to the disease or disorder. In some cases, the treatment can stabilize the disease or disorder. In some cases, the treatment can delay progression of the disease or disorder. In some instances, the treatment can cause regression of the disease or disorder. In some cases, a treatment's effect can be measured. In some cases, measurements can be compared before and after administration of the composition. For example, a subject can have medical images prior to treatment compared to images after treatment to show improvement in joint health. In some instances, a subject may have an improved blood test result after treatment compared to a blood test before treatment. In some cases, a measurement for improvement can include decreased pain (e.g. joint paint), improved mobility (e.g. joint mobility), decreased deterioration (e.g. joint deterioration), and decreased inflammation (e.g. joint inflammation) relative to the state of the joint prior to treatment. In some instances, measurements can be compared to a standard.

In some embodiments, a subject can have a disease, a disorder, a condition or any combination thereof. In some cases, a subject can have joint pain, joint inflammation, joint immobilization, joint deterioration or any combination thereof. In some cases, a disease can comprise arthritis. In some instances, arthritis can be chronic arthritis or acute arthritis. Chronic arthritis can comprise rheumatoid arthritis, psoriatic arthritis, childhood arthritis, lupus, erythematosus, osteoarthritis, degenerative arthritis, juvenile idiopathic arthritis, fibromyalgia, polymyalgia rheumatica or any combination thereof. Acute arthritis can comprise: arthritis from an infection (e.g. Lyme disease, Hepatitis B, bacterial arthritis, viral arthritis, parasitic arthritis, fungal arthritis), tennis elbow, tarsal tunnel syndrome, gout, calcium pyrophosphate arthritis, bursitis, tendinitis or any combination thereof. In some embodiments, arthritis can comprise: spinal arthritis, reactive arthritis, ankylosing spondylitis, sacroiliitis, polymyalgia rheumatica, Behcet's syndrome, carpal tunnel syndrome, degenerative joint disease, muscular dystrophy, Ethlers-Danlos syndrome, Whipple's disease, Fabry's disease, osteochondromatosis, osteogenesis imperfecta, fibromyalgia, osteoporosis, plantas fasciitis, inflammatory arthritis or any combination thereof. Arthritis can be swelling and tenderness in one or more joints and can comprise pain, stiffness or both in one or more joints. In some instances, symptoms of arthritis can comprise joint stiffness, a tender joint, locking of a joint, a warm joint, a swollen joint, redness of a joint, fatigue, gland swelling (e.g. swollen lymph nodes) fever, loss of appetite, weight loss, fatigue or any combination thereof. In some instances, symptoms of arthritis can comprise limping, weakness, or both. In some cases, arthritis can be a loss of cartilage for example in osteoarthritis. In some instances, arthritis can comprise inflammation in a joint for example in rheumatoid arthritis.

As described herein, the composition can be administered to supplement the diet of a subject. In some cases, the composition can comprise a dietary supplement. The composition can comprise a dietary ingredient, such as, a vitamin, a mineral, an enzyme, an amino acid, an herb, a botanical or another substance that can supplement the diet of an individual. In some cases, a dietary supplement can boost energy or help maintain health of an individual.

A composition described herein can be administered as a therapy to treat or prevent a disease, such as arthritis.

A composition described herein, can be applied as a cosmetic. In some cases, a cosmetic can be a composition that can be intended to be rubbed, poured, sprinkled, sprayed on, introduced onto or otherwise applied to the human body for cleansing, beautifying, promoting attractiveness or altering the appearance. A cosmetic can comprise a moisturizer (e.g. a skin moisturizer), a perfume, a lipstick, a fingernail polish, a makeup (e.g. eye makeup, face makeup), a shampoo, a hair color, a deodorant, a cologne, a fragrant oil, or any composition that can be applied as a cosmetic product.

The section headings used herein are for organizational purposes and are not to be construed as limiting the subject matter described.

### Compositions

In some embodiments, disclosed herein can be a composition that can comprise cannabidiol (CBD), flavanone or salt thereof, and one or more of: a glucosamine, a salt thereof, a glycosaminoglycan, a salt thereof, a vitamin. a derivative thereof, a salt thereof or methylsulfonylmethane (MSM). In some cases, a composition described herein can further comprise a monoterpene, a derivative thereof, a salt thereof, a mineral, a salt thereof, or a chelated form thereof, an amino acid, a salt thereof, at least a portion of a plant or an extract thereof, an oil, a fatty acid, or any combination thereof. In some cases, the composition can comprise a sugar, sucralose, aspartame, stevia, or any combination thereof.

The composition described herein can be comprised in the form of a capsule, a tablet, a gummy, a food, a drink, an oil, a tincture, a lotion, a cream, a balm, a candy, a chocolate, or any combination thereof. In some instances, the composition described herein can be a dietary supplement and can be in the form of a capsule, a tablet, a gummy, a candy, a chocolate, a beverage, or any combination thereof. In some instances, the composition described herein can be a cosmetic. In some instances, the composition described herein can be pharmaceutical formulation.

In some embodiments, THC (tetrahydrocannabinol) can comprise an isoform of THC. In some instances, a composition described herein can comprise an isoform of THC (e.g. Δ8-THC). In some cases, a THC isoform can comprise Δ7-THC, Δ8-THC, Δ9-THC, Δ10-THC, Δ11-THC, or Δ13-THC. In some cases, a composition may not comprise THC. In some cases, a composition may comprise THC. In some cases, THC can comprise tetrahydrocannabivarin. In some cases, THC can comprise tetrahydrocannabinolic acid. In some cases, a composition described herein can comprise Δ8-THC. In some instances, THC can be comprised in a composition described herein in amounts (milligram) of more than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 750, 1000, 2000, 3000, 4000, or 5000 mg. In some instances, THC can be comprised in a composition described herein in amounts (milligram) of less than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 750, 1000, 2000, 3000, 4000, or 5000 mg. In some instances, THC can be comprised in a composition described herein in amounts (milligram) from about: 1 mg to about 100 mg, 1 mg to about 25 mg, 5 mg to about 30 mg, 10 mg to about 50 mg, 15 mg to about 75 mg, 50 mg to about 100 mg, about 75 mg to about 500 mg, about 250 mg to about 1500 mg, about 1000 mg to about 3000 mg, or about 1500 mg to about 5000 mg. In some cases, a composition may not comprise a substantial amount of Δ9-THC. For example, the composition may comprise less than about: 1%, 0.50%, 0.40%, 0.30%, 0.20%, 0.10%, 0.05%, 0.03%, 0.02%, 0.01%, 0.009%, 0.005%, 0.001%, or 0.0001% Δ9-THC. In some cases, the composition may comprise more than about: 1%, 0.50%, 0.40%, 0.30%, 0.20%, 0.10%, 0.05%, 0.01%, 0.009%, 0.005%, 0.001%, or 0.0001% Δ9-THC. In some cases, the composition can comprise from about: 0.0001% Δ9-THC to about 1.0% Δ9-THC, 0.0001% Δ9-THC to about 0.02% Δ9-THC, 0.001% Δ9-THC to about 0.02% Δ9-THC, 0.01% Δ9-THC to about 0.30% Δ9-THC, 0.01% Δ9-THC to about 0.10% Δ9-THC, 0.10% Δ9-THC to about 0.50% Δ9-THC, 0.1% Δ9-THC to about 1.0% Δ9-THC, or from about 0.20% Δ9-THC to about 0.80% Δ9-THC. In some cases, the percentages of THC can be with respect to a total weight or a total volume of a composition. In some instances, THC can comprise trans-THC, cis-THC or both. In some cases, THC can exist as a stereoisomer, such as, (+)-trans-THC; (−)-trans-THC; (+)-cis-THC and (−)-cis-THC. In some cases, cis-TCH can comprise, (+)-cis-THC, (−)-cis-THC, or both. In some cases, trans-THC can comprise (+)-trans-THC, (−)-trans-THC, or both. In some cases, a composition can comprise a ratio (weight to weight) of THC to CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of CBD to THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of trans-THC to cis-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of cis-THC to trans-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. THC can comprise an isomer. In some cases, CBD can be mixed in a composition with Δ8-THC, Δ9-THC, Δ10-THC or a combination thereof. In some cases, a composition can comprise a ratio (weight to weight) of Δ8-THC, Δ9-THC, or Δ10-THC to CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of CBD to Δ8-THC, Δ9-THC, or Δ10-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some instances, THC can comprise an enantiomer, or a diastereomer. In some instances, THC can be a racemate.

In some embodiments, CBD can comprise cannabidiol, cannabidiolic acid or any combination thereof. In some instances, CBD can be comprised in a composition described herein in amounts (milligram) of more than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 750, 1000, 2000, 3000, 4000, or 5000 mg. In some instances, CBD can be comprised in a composition described herein in amounts (milligram) of less than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 750, 1000, 2000, 3000, 4000, or 5000 mg. In some instances, CBD can be comprised in a composition described herein in amounts (milligram) from about: 1 mg to about 100 mg, 1 mg to about 25 mg, 5 mg to about 30 mg, 10 mg to about 50 mg, 15 mg to about 75 mg, 50 mg to about 100 mg, about 75 mg to about 500 mg, about 250 mg to about 1500 mg, about 1000 mg to about 3000 mg, or about 1500 mg to about 5000 mg. In some cases, CBD can comprise an isomer. In some cases, CBD can comprise trans-CBD. In some cases, trans-CBD can comprise (+)-trans-CBD, (−)-trans-CBD, or both. In some instances, CBD can comprise an enantiomer, or a diastereomer. In some instances, CBD can comprise a racemate. In some instances, CBD can comprise trans-CBD, cis-CBD or both. In some cases, CBD can comprise (1R,6R)-CBD, (1R,6S)-CBD, (1S,6R)-CBD, (1S,6S)-CBD, or a combination thereof. In some cases, a composition can comprise a ratio (weight to weight) of trans-CBD to cis-CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of cis-CBD to trans-CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some instances, CBD can be a powder, a liquid, an oil, an emulsion, an aerosol, a solid or a combination thereof. In some cases, CBD can be at least partially water soluble.

In some embodiments, a composition herein can comprise a cannabinoid. In some cases, a cannabinoid can be in oil form. In some cases, a cannabinoid can be at least partially water soluble. In some cases, a cannabinoid may include any of the identified cannabinoids, but not limited to Tetrahydrocannabinol (THC), Tetrahydrocannabinolic acid (THCA); Cannabidiol (CBD); Cannabidiolic Acid (CBDA); Cannabinol (CBN); Cannabigerol (CBG); Cannabichromene (CBC); Cannabicyclol (CBL); Cannabivarin (CBV); Tetrahydrocannabivarin (THCV); Cannabidivarin (CBDV); Cannabichromevarin (CBCV); Cannabigerovarin (CBGV); Cannabigerol Monomethyl Ether (CBGM); Cannabielsoin (CBE); Cannabicitran (CBT); 10-Oxo-delta-6a-tetrahydrocannabinol (OTHC); Cannabichromanon (CBCF); Cannabifuran (CBF); Cannabiglendol; Cannabiripsol; and Cannbicitran. In some cases, a cannabinoid can comprise a cannabinoid from Table 1. In some instances, a cannabinoid can comprise a phytocannabinoid. In some instances, a cannabinoid can comprise an endocannabinoid. In some instances, an endocannabinoid can comprise anandamide (arachidonoyl ethanolamide) or 2-arachidonoyl glycerol (2-AG). In some cases, a cannabinoid can be a full spectrum cannabinoid. In some cases, a cannabinoid can be a broad-spectrum cannabinoid. In some cases, a composition herein can comprise one or more cannabinoids. In some cases, a composition herein can comprise 1, 2, 3, 4, 5, 6, 7, 9, 10 or more cannabinoids.

TABLE 1
List of exemplary cannabinoids

| Cannabinoid | Abbreviation |
|---|---|
| Cannabigerovarin | CBGV |
| Cannabigerovarinic acid | CBGVA |
| Cannabigerol | CBG |
| Cannabigerolic acid | CBGA |
| O-Methlycannabigerol | |
| Cannabigerolic acid methylether | |
| Cannabinerolic acid | |
| Cannabinerol | |
| Carmagerol | |
| rac-6′-Epoxycannabigerol | |
| rac-6′-Epoxycannabigerolic acid | |
| rac-6′-Epoxycannabinerol | |
| rac-6′-Epoxycannabinerolic acid | |
| γ-Eudesmyl cannabigerolate | |
| γ-Cadinyl cannabigerolate | |
| Sesquicannabigerol | |
| Deprenyl O-methyl | |
| cannabigerolic acid (Amorfrutin 2) | |
| 5-Acetyl-4-hydroxycannabigerol | |
| Acetylcannabigeroquinol | |
| Cannabigeroquinone | |
| Abnormal cannabigerol | |
| Acetyl abnormal | |
| hydrocannabigeroquinol | |
| Abnormal cannabigeroquinol | |
| 2′-Hydroxy-1′,2′- | |
| dihydrocannabichromene | |
| (Cyclo-CBG) | |
| Cannabiorcichromene | |
| Cannabiorcichromenic acid | |
| Chlorcannabiorcichromenic acid | |
| Cannabivarichromene | CBCV |
| Cannabichromevarinic acid | |
| Cannabichromene | CBC |
| Cannabichromenic acid | |
| 4-Acetoxycannabichromene | |
| Anthopogochromenic acid | |
| Confluentin | |
| Daurichromenic acid | |
| 8′-Hydroxyisocannabichromene | |
| 4-Acetoxycannabichromene | |
| Cannabidiorcol | |
| Cannabidivarin | CBDV |
| Cannabidivarinic acid | |
| nor-Cannabidiol | |
| Cannabidiol | CBD |
| O-Methlycannabidiol | |
| O-Propylcannabidiol | |
| O-Pentylcannabidiol | |
| Cannabidiolic acid | CBDA |
| CBDA-THC ester | |
| Ferruginene C | |
| Cannabioxepane | |
| Cannabinodivarin | |
| Cannabinodiol | |
| Cannabifuran | |
| Dehydrocannabifuran | |
| trans-Δ-8-Tetrahydrocannabinol | |
| trans-Δ-8-Tetrahydrocannabinolic acid | |
| 10α-Hydroxytrans-Δ-8-tetrahydrocannabinol | |
| 10β-Hydroxytrans-Δ-8-tetrahydrocannabinol | |
| 11-Acetoxy-Δ-8-tetrahydrocannabinoic acid | |
| 10-Hydroxy-9-oxo-Δ-8-tetrahydrocannabinol | |
| Δ-9-trans-Tetrahydrocannabiorcol | |
| Δ-9-trans-Tetrahydrocannabiorcolic acid | |
| Δ-9-trans-Tetrahydrocannabivarin | |
| Δ-9-trans-Tetrahydrocannabivarinic acid | |
| Δ-9-trans-nor-Tetrahydrocannabinol | |
| Δ-9-trans-nor-Tetrahydrocannabinolic acid | |
| Δ-9-trans-Tetrahydrocannabinol | |
| Δ-9-trans-Tetrahydrocannabinolic acid A | |
| Δ-9-trans-Tetrahydrocannabinolic acid B | |
| 8α-Hydroxy-Δ-9-trans-tetrahydrocannabinol | |
| 8β-Hydroxy-Δ-9-trans-tetrahydrocannabinol | |
| 8-Oxo-Δ-9-trans-tetrahydrocannabinol | |
| O-Propyl-Δ-9-trans-tetrahydrocannabinol | |
| O-Pentyl-Δ-9-trans-tetrahydrocannabinol | |
| 2-Formyl-Δ-9-trans-tetrahydrocannabinol | |
| β-Fenchyl Δ-9-trans-Tetrahydrocannabinolate | |
| α-Fenchyl Δ-9-trans-Tetrahydrocannabinolate | |
| Bornyl Δ-9-trans-Tetrahydrocannabinolate | |
| epi-Bornyl Δ-9-trans-Tetrahydrocannabinolate | |
| α-Terpinyl Δ-9-trans-Tetrahydrocannabinolate | |
| 4-Terpinyl Δ-9-trans-Tetrahydrocannabinolate | |
| γ-Eudesmyl Δ-9-trans-Tetrahydrocannabinolate | |
| α-Cadinyl Δ-9-trans-Tetrahydrocannabinolate | |
| Hexahydrocannabinol | |
| Hydroxy Δ-9,11-hexahydrocannabinol | |
| Methylen-bis Δ-9-trans-Tetrahydrocannabinol | |
| (Cannabisol) | |
| Tetrahydrocannabinol epoxide | |
| Δ-9-trans-Tetrahydrocannabinol glycol | |
| (cannabiripsol) | |
| 6a,7,10a-Trihydroxy-Δ-9-tetrahydrocannabinol | |
| Δ-9-cis-Tetrahydrocannabivarin | |
| Δ-9-cis-Tetrahydrocannabinol | |
| Cannabicitran (citrilidene- cannabis) | CBT |
| Cannabiorcicitran | |
| Bis-nor cannabitriol | |
| Bis-nor-Cannabitriol isomer | |
| 10-O-Ethyl bis-nor cannabitriol | |
| Isocannabitriol | |
| Cannabitriol | |
| Cannabitriol isomer | |
| 10-O-Ethyl cannabitriol isomer | |
| 10-Oxo-Δ6a(10a)-tetrahydrocannabinol | |
| 9,10-Anhydrocannabitirol | |
| Cannabiglendol | |
| 7,8-Dehydro-10-O-ethylcannabitriol | |
| Δ-7-cis-Isotetrahydrocannabivarin | |
| Δ-7-trans-Isotetrahydrocannabivarin | |
| Δ-7-trans-isotetrahydrocannabinol | |
| Bis-nor-cannabielsoin | |
| Bis-nor-Cannabielsoic acid B | |
| Cannabielsoin | CBE |
| Cannabielsoic acid A | |
| Cannabielsoic acid B | |
| Ferruginene A | |
| Ferruginene B | |
| Cannabiorcicyclol | |
| Cannabiorcicyclolic acid | |
| Cannabicyclovarin | |
| Cannabicyclol | CBL |
| Cannabicyclolic acid | |
| Anthopogocyclolic acid | |
| Rhododaurichromanic acid A | |
| Cannabiorcol | |
| nor-Cannabivarin | |
| Cannabivarin | CBV |
| nor-Cannabinol | |
| Cannabinol | CBN |
| Cannabinolic acid | |
| O-Methylcannabinol | |
| O-Propylcannabinol | |
| O-Penthylcannabinol | |
| 7-Hydroxcannabinol | |
| 8-Hydroxycannabinol | |
| 8-Hydroxcannabinolic acid | |
| 7,8-Dihydrocannabinol | |
| 4-Terpenyl cannabinolate | |
| Cannabicoumaronone | |
| Cannabicourmarononic acid | |
| Bisnor-Cannabichromanone | |
| Cannabichromanone | |
| (6aR)-Cannabichromanone B | |
| (6aR)-Cannabichromanone C | |
| Cannabichromanone | |
| Cannabimovone | |
| Anhydrocannabimovone | |
| Demethyldecarboxyamorfrutin A | |
| Tetrahydrocannabiphorol | THCP |
| Cannabidiphorol | CBDP |
| Cannabimovone | CBM |

In some embodiments, a cannabinoid can be comprised in a composition described herein in amounts (milligram) of more than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 750, 1000, 2000, 3000, 4000, or 5000 mg. In some instances, a cannabinoid can be comprised in a composition described herein in amounts (milligram) of less than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 750, 1000, 2000, or 3000, 4000, or 5000 mg. In some instances, a cannabinoid can be comprised in a composition described herein in amounts (milligram) from about: 1 mg to about 100 mg, 1 mg to about 25 mg, 5 mg to about 30 mg, 10 mg to about 50 mg, 15 mg to about 75 mg, 50 mg to about 100 mg, about 75 mg to about 500 mg, about 250 mg to about 1500 mg, about 1000 mg to about 3000 mg, or about 1500 mg to about 5000 mg. In some instances, a cannabinoid can comprise an isomer. In some instances, a cannabinoid can comprise an enantiomer, or a diastereomer. In some instances, a cannabinoid can be a racemate.

In some embodiments, a flavanone can comprise naringenin, a salt thereof, naringin, a salt thereof, or any combination thereof. In some cases, a flavanone can comprise a derivative of naringenin, naringin, a flavanone or a salt thereof. In some instances, a flavanone can comprise blumeatin, butin, eriodictyol, hesperetin, hesperidin, homoeriodictyol, isosakuranetin, pinocembrin, poncirin, sakuranetin, sakuranin, sterubin, pinostrobin or any combination thereof. In some cases, a composition herein can comprise 1, 2, 3, 4, 5, 6, 7, 9, 10 or more flavanones. In some instances, a flavanone can be in the form of an extract. For example, a flavanone can be an extract from a grapefruit, a bergamot, a sour orange, a tart cherry, a citrus fruit, a tomato, a cocoa, a Greek oregano, a water mint, a drynaria, a bean, a plant or any combination thereof. In some instances, naringenin, naringin, or a salt thereof can be comprised in a composition described herein in amounts (milligram) of more than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 250, 300, 350, 400, 450, 500 or 1000 mg. In some instances, naringenin, naringin or a salt thereof can be comprised in a composition described herein in amounts of less than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 250, 300, 350, 400, 450, 500 or 1000 mg. In some instances, naringenin, naringin or a salt thereof can be comprised in a composition described herein in amounts from about: 1 mg to about 100 mg, 1 mg to about 25 mg, 5 mg to about 30 mg, 10 mg to about 50 mg, 15 mg to about 75 mg, 50 mg to about 100 mg, about 75 mg to about 500 mg or about 250 mg to about 1000 mg.

In some embodiments, glucosamine can comprise glucosamine sulfate. A glucosamine can comprise an amino sugar. In some cases, glucosamine can comprise a salt of glucosamine (e.g. glucosamine sulfate, glucosamine chloride). In some instances, glucosamine can comprise glucosamine chondroitin, glucosamine hydrochloride, N-acetylglucosamine or a combination thereof. In some instances, a glucosamine can be comprised in a composition described herein in amounts (milligram) of more than about: 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or 5000 mg. In some instances, a glucosamine can be comprised in a composition described herein in amounts of less than about: 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or 5000 mg. In some instances, a glucosamine can be comprised in a composition described herein in amounts from about: 50 mg to about 500 mg, 200 mg to about 800 mg, 300 mg to about 600 mg, 400 mg to about 1000 mg, 600 mg to about 1500 mg, 1000 mg to about 2000 mg, or about 1500 mg to about 5000 mg.

In some embodiments, a composition herein can comprise a vitamin, a derivative thereof or a salt thereof. In some embodiments, a vitamin can comprise vitamin A, vitamin C, vitamin D, vitamin E, vitamin D, vitamin B1, vitamin B2, vitamin B3, vitamin B5, vitamin B6, vitamin B7, vitamin B9, vitamin B12, a derivative of any of these, a salt of any of these, or any combination thereof. In some cases, vitamin C can be a salt. For example, vitamin C can comprise sodium ascorbate, calcium ascorbate, potassium ascorbate, magnesium ascorbate, zinc ascorbate, molybdenum ascorbate, chromium ascorbate, manganese ascorbate or any combination thereof. A vitamin can be fat soluble or water soluble. For example, a vitamin can be comprised in an oil or in an aqueous solution. In some cases, a vitamin can be a powder or a solid. In some cases, a composition can comprise a metabolite of a vitamin, for example, dehydroascorbic acid. In some instances, a metabolite can comprise a derivate of a vitamin or a compound disclosed herein. In some cases, a composition described herein can comprise a choline, carnitine or both. In some cases, a vitamin can comprise vitamin C or a salt thereof (e.g. calcium ascorbate.) In some instances, vitamin C can be comprised in a composition described herein in amounts (milligram) of more than about: 5, 10, 15, 20, 25, 30, 40, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or 5000 mg. In some instances, vitamin C can be comprised in a composition described herein in amounts of less than about: 5, 10, 15, 20, 25, 30, 40, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or 5000 mg. In some instances, vitamin C can be comprised in a composition described herein in amounts from about: 5 mg to about 30 mg, 10 mg to about 60 mg, 20 mg to about 100 mg, 50 mg to about 500 mg, 200 mg to about 800 mg, 300 mg to about 600 mg, 400 mg to about 1000 mg, 600 mg to about 1500 mg, 1000 mg to about 2000 mg, or about 1500 mg to about 5000 mg. In some cases, a composition herein can comprise 1, 2, 3, 4, 5, 6, 7, 9, 10 or more vitamins.

In some embodiments, a glycosaminoglycan can comprise chondroitin sulfate, dermatan sulfate, keratan sulfate, hyaluronan, a salt thereof, or any combination thereof. In some cases, a composition herein can comprise 1, 2 or more glycosaminoglycans. In some cases, glycosaminoglycan can be linear polysaccharides that can have repeating disaccharide units. In some cases, a composition described herein can comprise chondroitin sulfate at a concentration (milligram) of more than about: 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or 5000 mg. In some cases, a composition described herein can comprise chondroitin sulfate at a concentration of less than about: 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 mg, or 5000 mg. In some instances, chondroitin sulfate can be comprised in a composition described herein in amounts from about: 5 mg to about 300 mg, 10 mg to about 650 mg, 20 mg to about 100 mg, 50 mg to about 500 mg, 200 mg to about 800 mg, 300 mg to about 650 mg, 400 mg to about 1000 mg, 550 mg to about 1500 mg, 1000 mg to about 2000 mg, or about 1500 mg to about 5000 mg.

In some embodiments, a composition described herein can comprise a monoterpene, for example D-limonene or a salt thereof. In some instances, D-limonene can be comprised in a composition described herein in amounts (micrograms) of more than about: 10, 20, 30, 40, 50, 60, 70, 80, 90, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or 5000 micrograms (mcg). In some instances, D-limonene can be comprised in a composition described herein in amounts of less than about; 10, 20, 30, 40, 50, 60, 70, 80, 90, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or 5000 mcg. In some instances, D-limonene can be comprised in a composition described herein in amounts from about: 10 mcg to about 200 mcg, 25 mcg to about 125 mcg, 100 mcg to about 300 mcg, 50 mcg to about 500 mcg, 200 mcg to about 800 mcg, 300 mcg to about 600 mcg, 400 mcg to about 1000 mcg, 600 mcg to about 1500 mcg, 1000 mcg to about 2000 mcg, or about 1500 mcg to about 5000 mcg. In some instances, a composition herein can comprise a monoterpene, a derivative thereof, a salt thereof, or any combination thereof. In some instances, a monoterpene can comprise two isoprene units and have a molecular formula C₁₀H₁₆. In some cases, a derivative can comprise a structurally related compound to a monoterpene, a metabolite of a monoterpene, or both. In some cases, a monoterpene can comprise myrcene, menthol, nerol, pinene, camphor, myrcene, phelladrene, terpinene, pinene, camphene, citronellol, carvacrol, thymol, citronellal, thujone, limonene, carvone, linalool or any combination thereof. In some instances, a monoterpene can exist as an enantiomer, such as a D or and L enantiomer. For example, limonene can comprise a D-enantiomer or an L-enantiomer. In some cases, a monoterpene can exist as an isomer, for example α-pinene and β-pinene.

In some embodiments, a composition described herein can comprise methylsulfonylmethane (MSM). MSM can be known as methyl sulfone and dimethyl sulfone. In some instances, MSM can be comprised in a composition described herein in amounts (milligram) of more than about: 5, 10, 20, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, or 8000 mg. In some instances, MSM can be comprised in a composition described herein in amounts of less than about: 5, 10, 20, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, or 8000 mg. In some instances, MSM can be comprised in a composition described herein in amounts from about: 5 mg to about 100 mg, 50 mg to about 500 mg, 200 mg to about 800 mg, 300 mg to about 600 mg, 400 mg to about 1000 mg, 600 mg to about 1500 mg, 1000 mg to about 2000 mg, 1500 mg to about 3000 mg, 1750 mg to about 4000 mg, or about 2000 mg to about 8000 mg.

In some embodiments, a composition herein can comprise a mineral, a salt thereof, or a chelated form thereof. In some cases, a mineral can comprise: a calcium, a phosphorus, a potassium, a sulfur, a sodium, a chloride, a magnesium, an iron, a zinc, a copper, a manganese, an iodine, a selenium, a molybdenum, a chromium, a fluoride, or any combination thereof. A chelated form of a mineral can comprise copper chelate, potassium iodine, di-magnesium malate, magnesium bisglycinate chelate, zinc bisglycinate chelate, selenium glycinate, copper bisglycinate chelate, manganese bisglycinate chelate, chromium chelate, molybdenum glycinate chelate, potassium glycinate chelate or any combination thereof. In some instances, a mineral can comprise sodium chloride.

In some embodiments, a composition described herein can comprise a terpene, for example β-myrcene, β-caryophyllene, ocimene, α-pinene, α-humulene, linalool, p-cymene, camphene, cis-nerolidol, terpinolene, isopulegol, caryophyllene oxide, δ-limonene, geraniol, guaiol, α-bisabolol, 3-carene, β-pinene, γ-terpinene, or a salt of any of these. In some instances, a terpene can be comprised in a composition described herein in amounts (micrograms) of more than about: 10, 20, 30, 40, 50, 60, 70, 80, 90, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or 5000 micrograms (mcg). In some instances, a terpene can be comprised in a composition described herein in amounts of less than about; 10, 20, 30, 40, 50, 60, 70, 80, 90, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or 5000 mcg. In some instances, a terpene can be comprised in a composition described herein in amounts from about: 10 mcg to about 200 mcg, 25 mcg to about 125 mcg, 100 mcg to about 300 mcg, 50 mcg to about 500 mcg, 200 mcg to about 800 mcg, 300 mcg to about 600 mcg, 400 mcg to about 1000 mcg, 600 mcg to about 1500 mcg, 1000 mcg to about 2000 mcg, or about 1500 mcg to about 5000 mcg. In some instances, a terpene can be comprised in a composition described herein in amounts (milligrams) of more than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 milligrams. In some instances, a terpene can be comprised in a composition described herein in amounts (milligrams) of less than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 milligrams. In some instances, a composition herein can comprise a terpene, a derivative thereof, a salt thereof, or any combination thereof. In some cases, a terpene can comprise a terpene in Table 2, a derivative thereof or a salt thereof.

TABLE 1
List of exemplary terpenes

| | | RI | | |
|---|---|---|---|---|
| No. | chemical name | (DB1) | formula | MW |
| 1 | Fusicocca-3,5-diene | 1850 | C20H32 | 272 |
| 2 | 9-epi-Sclarene | 1896 | C20H32 | 272 |
| 3 | Laurenene | 1903 | C20H32 | 272 |
| 4 | Rimuene | 1907 | C20H32 | 272 |
| 5 | Isopimara-8,15-diene | 1922 | C20H32 | 272 |
| 6 | Cembrene | 1938 | C20H32 | 272 |
| 7 | Pimara-8,15-diene | 1942 | C20H32 | 272 |
| 8 | Sclarene | 1943 | C20H32 | 272 |
| 9 | Isohibaene | 1944 | C20H32 | 272 |
| 10 | Rosa-5,15-diene | 1945 | C20H32 | 272 |
| 11 | (E)-2,6-Dimethyl-10-(p-tolyl)- | 1945 | C20H30 | 270 |
| | undeca-2,6-diene | | | |
| 12 | Isocembrene | 1951 | C20H32 | 272 |
| 13 | Beyerene | 1951 | C20H32 | 272 |
| 14 | Pimara-8(14),15-diene | 1955 | C20H32 | 272 |
| 15 | Cembrene A | 1962 | C20H32 | 272 |
| 16 | Labda-7,13,14-triene | 1978 | C20H32 | 272 |
| 17 | Isopimara-8(14),15-diene | 1981 | C20H32 | 272 |
| 18 | Isophyllocladene | 1982 | C20H32 | 272 |
| 19 | Dolabella-6,10,15-triene | 1984 | C20H32 | 272 |
| 20 | (Z)-Biformene | 1988 | C20H32 | 272 |
| 21 | Manool oxide | 2007 | C20H34O | 290 |
| 22 | Geranyllinalool | 2008 | C20H32 | 272 |
| 23 | Isopimara-7,15-diene | 2010 | C20H32 | 272 |
| 24 | 15-Kaurene | 2011 | C20H32 | 272 |
| 25 | Isopimara-8,15-diene | 2016 | C20H32 | 272 |
| 26 | Dolabradiene | 2017 | C20H32 | 272 |
| 27 | Trachylobane | 2022 | C20H32 | 272 |
| 28 | (E)-Biformene | 2017 | C20H32 | 272 |
| 29 | Cembrene C | 2023 | C20H32 | 272 |
| 30 | 13-epi-Manoyl oxide | 2023 | C20H34O | 290 |
| 31 | (E)-Labda-7,12,14-triene | 2036 | C20H32 | 272 |
| 32 | Phyllocladene | 2042 | C20H32 | 272 |
| 33 | Abietatriene | 2046 | C20H30 | 270 |
| 34 | 16-Atisirene | 2051 | C20H32 | 272 |
| 35 | 16-Kaurene | 2056 | C20H32 | 272 |
| 36 | Manool | 2070 | C20H32 | 272 |
| 37 | Aphidicol-15-ene | 2073 | C20H32 | 272 |
| 38 | Valpara-2,15-diene | 2073 | C20H32 | 272 |
| 39 | Abieta-7,13-diene | 2084 | C20H32 | 272 |
| 40 | Labda-7,14-dien-13-ol | 2096 | C20H34O | 290 |
| 41 | Aphidicol-16-ene | 2102 | C20H32 | 272 |
| 42 | Isoabienol | 2124 | C20H34O | 290 |
| 43 | Abieta-8(14),13(15)-diene | 2152 | C20H32 | 272 |
| 44 | (8a,12Z)-Abienol | 2146 | C20H34O | 290 |
| 45 | Incensole | 2193 | C20H34O2 | 306 |
| 46 | Sclareol | 2231 | C20H36O2 | 308 |
| 47 | Labda-8(17),14-dien-6,13-diol | 2248 | C20H34O2 | 306 |
| 48 | Incensol acetate | 2220 | C22H36O3 | 348 |
| 49 | Verticilla-4(20),7,11-triene | 2040 | C20H32 | 272 |
| 50 | m-Camphorene | 1947 | C20H32 | 272 |
| 51 | p-Camphorene | 1980 | C20H32 | 272 |
| 52 | Cembrenol | 2131 | C20H34O2 | 290 |
| 53 | Sterna-13-ene | 2025 | C20H32 | 272 |
| 54 | 2-Allyl-4-methylphenol | 1262 | C10H12O | 148 |
| 55 | 8,9-Dehydrothymol acetate | 1360 | C12H14O2 | 190 |
| 56 | 3,5,5-Trimethyl-4- | 1200 | C10H14O | 150 |
| | methylenecyclohex-2-enone | | | |
| 57 | Cabreuva oxide D | 1467 | C15H24O | 220 |
| 58 | p-Isopropylbenzaldehyd | 1220 | C10H12O | 148 |
| 59 | 3-Methyl-4-(2,6,6- | 1471 | C14H22O | 206 |
| | trimethylcyclohex-2-enyl)-but-3-en- | | | |
| | 2-one | | | |
| 60 | 2,6,6-Trimethyl-3-oxocyclohex-1- | 1110 | C10H14O2 | 166 |
| | ene-1-carbaldehyde | | | |
| 61 | Isophorone | 1100 | C9H14O | 138 |
| 62 | 3,5,5-Trimethylcyclohex-3-enone | 1027 | C9H14O | 138 |
| 63 | trans-Sabinyl acetate | 1278 | C12H16O2 | 192 |
| 64 | Nerol | 1210 | C10H18O | 154 |
| 65 | 3a-Hydroxy-1,8-cineol | 1217 | C10H18O2 | 170 |
| 66 | Carvone | 1214 | C10H14O | 150 |
| 67 | Thymol methyl ether | 1215 | C11H16O | 164 |
| 68 | Pulegone | 1215 | C10H16O | 152 |
| 69 | Neral | 1215 | C10H16O | 152 |
| 70 | p-Anisaldehyde | 1218 | C8H8O2 | 136 |
| 71 | Chavicol | 1219 | C9H10O | 134 |
| 72 | 2,3-Dehydro-1,4-cineol | 1219 | C10H16O | 152 |
| 73 | trans-Isopulegone | 1161 | C101116O | 152 |
| 74 | Piperitone | 1226 | C10H16O | 152 |
| 75 | 1,4-Dimethoxy-2-methylbenzene | 1226 | C9H12O2 | 152 |
| 76 | Carvacrol methyl ether | 1226 | C11H16O | 164 |
| 77 | Isobornyl formate | 1228 | C11H18O2 | 182 |
| 78 | 2-Phenylethyl acetate | 1230 | C10H12O2 | 164 |
| 79 | (E)-Cinnamaldehyde | 1234 | C9H8O | 132 |
| 80 | 2,3-Dehydro-1,8-cineol | 993 | C10H16O | 152 |
| 81 | 2-Hydroxypinan-3-one | 1235 | C10H16O2 | 168 |
| 82 | Geraniol | 1235 | C10H18O | 154 |
| 83 | Pseudodiosphenol | 1245 | C10H16O2 | 168 |
| 84 | cis-Chrysanthenyl acetate | 1253 | C12H18O2 | 194 |
| 85 | trans-Carvone epoxide | 1243 | C10H14O2 | 166 |
| 86 | cis-Sabinene hydrat acetate | 1248 | C12H20O2 | 196 |
| 87 | cis-Ethyl chrysanthemate | 1251 | C12H20O2 | 196 |
| 88 | trans-Sabinen hydrate acetate | 1254 | C12H20O2 | 196 |
| 89 | Citronellyl formate | 1259 | C11H20O2 | 184 |
| 90 | Perilla aldehyde | 1260 | C10H14O | 150 |
| 91 | trans-Ethyl chrysanthemate | 1260 | C12H20O2 | 196 |
| 92 | trans-Anethol | 1262 | C10H12O | 148 |
| 93 | Nonanoic acid | 1263 | C9H18O2 | 158 |
| 94 | Isopulegol acetate (Isomer 1) | 1263 | C12H20O2 | 196 |
| 95 | Methyl nerolate | 1265 | C11H18O2 | 182 |
| 96 | Safrol | 1265 | C10H10O2 | 162 |
| 97 | cis-Thiorose oxide | 1265 | C10H18S | 170 |
| 98 | cis-Verbenyl acetate | 1266 | C12H18O2 | 194 |
| 99 | Thymol | 1267 | C10H14O | 150 |
| 100 | Bornyl acetate | 1270 | C12H20O2 | 196 |
| 101 | Neomenthyl acetate | 1263 | C12H22O2 | 198 |
| 102 | Deca-2,4-dienal | 1270 | C10H16O | 152 |
| 103 | Isopulegol acetate (Isomer 2) | 1271 | C12H20O2 | 196 |
| 104 | 2-Undecanone | 1273 | C10H16O | 152 |
| 105 | 4,8-Dimethylnonanol | 1276 | C11H124O | 172 |
| 106 | Diosphenol | 1276 | C10H16O2 | 168 |
| 107 | Isobornyl acetate | 1276 | C12H20O2 | 196 |
| 108 | Carvacrol | 1278 | C10H14O | 150 |
| 109 | Thujopsadiene | 1470 | C15H22 | 202 |
| 110 | Sesamol | 1280 | C7H6O3 | 138 |
| 111 | Menthyl acetate | 1280 | C12H22O2 | 198 |
| 112 | Geranial | 1244 | C10H16O | 152 |
| 113 | Geranyl formate | 1284 | C11H18O2 | 182 |
| 114 | trans-Thiorose oxide | 1284 | C10H18S | 170 |
| 115 | 2-Undecanol | 1284 | C11H24O | 172 |
| 116 | p-Isopropylbenzyl alcohol | 1285 | C10H14O | 150 |
| 117 | Sencyunolide | 1672 | C12H16O2 | 192 |
| 118 | trans-Pinocarvyl acetate | 1287 | C12H18O2 | 194 |
| 119 | 2,3,6-Trimethylbenzaldehyde | 1287 | C10H12O | 148 |
| 120 | Terpinen-4-ol acetate | 1289 | C12H20O2 | 196 |
| 121 | Chrysanthenone epoxide | 1290 | C10H14O2 | 166 |
| 122 | (E,E)-Deca-2,4-dienal | 1291 | C10H16O | 152 |
| 123 | Puleganolide (Isomer 1) | 1292 | C10H16O2 | 168 |
| 124 | Dihydrocarveol acetate (Isomer 2) | 1295 | C12H20O2 | 196 |
| 125 | Isoascaridol | 1295 | C10H16O2 | 168 |
| 126 | Theaspirane (Isomer 1) | 1299 | C13H22O | 194 |
| 127 | cis-Pinocarvyl acetate | 1300 | C12H18O2 | 194 |
| 128 | Dihydronaginata ketone | 1300 | C10H14O2 | 166 |
| 129 | Naginata ketone alcohol | 1306 | C10H14O3 | 182 |
| 130 | Puleganolide (Isomer 2) | 1305 | C10H16O2 | 168 |
| 131 | Methyl geranate | 1306 | C11H18O2 | 182 |
| 132 | Vinylguaiacol | 1311 | C9H10O2 | 150 |
| 133 | 5-Acetoxylinalool | 1303 | C12H20O3 | 212 |
| 134 | Theaspirane (Isomer 2) | 1313 | C13H22O | 194 |
| 135 | Chavicol acetate | 1313 | C11H12O2 | 176 |
| 136 | Myrtenyl acetate | 1313 | C12H18O2 | 194 |
| 137 | Dihydrocarveol acetate (Isomer 2) | 1314 | C12H20O2 | 196 |
| 138 | Apiol | 1649 | C12H14O4 | 222 |
| 139 | trans-Carvyl acetate | 1318 | C12H18O2 | 194 |
| 140 | Thymol acetate | 1329 | C12H16O2 | 192 |
| 141 | Menthothiophene | 1330 | C10H14S | 166 |
| 142 | 2,3,4-Trimethylbenzaldehyde | 1331 | C10H12O | 148 |
| 143 | Eugenol | 1331 | C10H12O2 | 164 |
| 144 | cis-Dihydrocarvone epoxide | 1333 | C10H16O2 | 168 |
| 145 | Ethyl nerolat | 1335 | C12H20O2 | 196 |
| 146 | Fragranol | 1201 | C12H20O2 | 196 |
| 147 | 7,8-Dihydro-b-ionone | 1422 | C13H22O | 194 |
| 148 | 8-Hydroxylinalool | 1336 | C10H18O2 | 170 |
| 149 | 3,4-Dimethoxystyrene | 1337 | C10H12O2 | 164 |
| 150 | Citronellyl acetate | 1337 | C12H22O2 | 198 |
| 151 | trans-8-Mercapto-p-menthan-3-one | 1340 | C10H18OS | 186 |
| 152 | Anhydroencecalinol | 1640 | C14H16O2 | 216 |
| 153 | Neryl acetate | 1342 | C12H20O2 | 196 |
| 154 | Dihydrocarveol acetate (Isomer 2) | 1342 | C12H20O2 | 196 |
| 155 | exo-Isocamphanyl acetate | 1345 | C12H20O2 | 196 |
| 156 | cis-Carvyl acetate | 1345 | C12H18O2 | 194 |
| 157 | (Z)-Ethyl cinnamate | 1344 | C11H12O2 | 176 |
| 158 | Chavibetol (m-Eugenol) | 1346 | C10H12O2 | 164 |
| 159 | 4-Methoxyphenylethanol | 1347 | C9H12O2 | 152 |
| 160 | (E)-Anethol epoxide | 1347 | C10H12O2 | 164 |
| 161 | trans-Dihydrocarvone epoxide | 1352 | C10H16O2 | 168 |
| 162 | endo-Isocamphanyl acetate | 1352 | C12H20O2 | 196 |
| 163 | (E)-Methyl cinnamate | 1354 | C10H10O2 | 162 |
| 164 | cis-8-Mercapto-p-menthan-3-one | 1356 | C10H18OS | 186 |
| 165 | Dihydrojasmone | 1361 | C10H14O2 | 166 |
| 166 | (E)-b-Damascenone | 1363 | C13H18O | 190 |
| 167 | 3-Allyl-1,4-dimethoxybenzene | 1370 | C11H14O2 | 178 |
| 168 | (Z)-Jasmone | 1371 | C11H16O | 164 |
| 169 | Isobornyl propionate | 1375 | C13H22O2 | 210 |
| 170 | Ethyl geranate | 1377 | C12H20O2 | 196 |
| 171 | Methyl perillate | 1381 | C11H16O2 | 180 |
| 172 | (Z)-Isoeugenol | 1381 | C10H12O2 | 164 |
| 173 | Osmorhizol | 1383 | C11H14O2 | 178 |
| 174 | 2-Dodecanol | 1387 | C12H26O | 186 |
| 175 | 1-Tetradecene | 1387 | C14H28 | 196 |
| 176 | Davanafuran | 1394 | C14H20O2 | 220 |
| 177 | Methyl 4-methoxyphenylacetate | 1398 | C10H12O3 | 180 |
| 178 | (E)-b-Damascone | 1398 | C13H20O | 192 |
| 179 | trans-Carvyl propionate | 1402 | C13H20O2 | 208 |
| 180 | 2,6-Dimethoxycymene | 1402 | C12H18O2 | 194 |
| 181 | Nerylacetone | 1412 | C13H22O | 194 |
| 182 | 2-Hydroxy-1,2-dihydrolavandulyl | 1416 | C12H22O3 | 214 |
| | acetate | | | |
| 183 | (Z)-1,2-Dimethoxy-4- | 1419 | C11H14O2 | 178 |
| | propenylbenzene | | | |
| 184 | (E)-Cinnamyl acetate | 1420 | C11H12O2 | 176 |
| 185 | Isobornyl isobutyrate | 1424 | C14H24O2 | 224 |
| 186 | Citronellyl propionate | 1427 | C13H24O2 | 212 |
| 187 | 3,4-Dimethoxybenzaldehyde | 1428 | C9H10O3 | 166 |
| 188 | (E)-Isoeugenol | 1429 | C10H12O2 | 164 |
| 189 | cis-Carvyl propionate | 1436 | C13H20O2 | 208 |
| 190 | Massoialactone | 1439 | C10H16O2 | 168 |
| 191 | d-Undecanolide | 1565 | C11H20O2 | 184 |
| 192 | Nordavanone | 1451 | C11H18O2 | 182 |
| 193 | 8-Dehydrothymol isobutyrate | 1458 | C14H18O2 | 218 |
| 194 | (E)-1,2-Dimethoxy-4- | 1460 | C11H14O2 | 178 |
| | propenylbenzene | | | |
| 195 | Thymol isobutyrate | 1462 | C14H20O2 | 220 |
| 196 | Isobornyl butyrate | 1462 | CH4H24O2 | 224 |
| 197 | 3,4-Dimethoxybenzyl alcohol | 1464 | C9H12O3 | 168 |
| 198 | Sarisane | 1466 | C11H12O3 | 192 |
| 199 | 2-Tridecanone | 1477 | C13H26O | 198 |
| 200 | 2-Tridecanol | 1490 | C13H28O | 200 |
| 201 | Davana ether | 1489 | C15H22O2 | 234 |
| 202 | a-Campholenyl formate | 1240 | C11H18O2 | 182 |
| 203 | Chavibetyl acetate | 1488 | C12H14O3 | 206 |
| 204 | Homovanilline alcohol | 1494 | C9H12O3 | 168 |
| 205 | Davana ether (Isomer) | 1507 | C15H22O2 | 234 |
| 206 | Isobornyl isovalerate | 1516 | C15H26O2 | 238 |
| 207 | Citronellyl butyrate | 1516 | C14H26O2 | 226 |
| 208 | Elemicine | 1522 | C12H16O3 | 208 |
| 209 | Flavesone | 1526 | C14H20O4 | 252 |
| 210 | allo-Davanone | 1539 | C15H24O2 | 236 |
| 211 | Isodavanone | 1545 | C15H24O2 | 236 |
| 212 | Eupatoriochromene | 1726 | C13H16O3 | 220 |
| 213 | cis-Davanone | 1557 | C15H24O2 | 236 |
| 214 | Geranyl crotonate | 1555 | C14H22O2 | 222 |
| 215 | Diethylphthalate | 1555 | C12H14O4 | 222 |
| 216 | cis-8-Acetylthio-p-menthan-3-one | 1559 | C12H20O2S | 228 |
| 217 | 4-Allyl-2,6-dimethoxyphenol | 1561 | C11H14O3 | 194 |
| 218 | Sandela | 1568 | C16H28O | 236 |
| 219 | trans-8-Acetylthio-p-mentan-3-one | 1570 | C12H20O2S | 228 |
| 220 | (Z)-Asarone | 1584 | C12H16O3 | 208 |
| 221 | (Z)-3-Hexenyl benzoate | 1545 | C13H16O2 | 204 |
| 222 | Geranyl 2-methylbutyrate | 1591 | C15H26O2 | 238 |
| 223 | 1,2-Diacetoxy-4-allylbenzene | 1602 | C13H14O4 | 234 |
| 224 | Leptospermone | 1611 | C15H22O4 | 266 |
| 225 | (Z)-Ethyl p-methoxycinnamate | 1614 | C12H14O3 | 206 |
| 226 | Butylphthalide | 1616 | C12H14O2 | 190 |
| 227 | (E)-Asarone | 1636 | C12H16O3 | 208 |
| 228 | (Z)-Butylidenphthalide | 1644 | C12H12O2 | 188 |
| 229 | 6-Methoxythymol isobutyrate | 1658 | C15H22O3 | 250 |
| 230 | 2-Pentadecanone | 1688 | C15H30O | 226 |
| 231 | (E)-Ethyl p-methoxycinnamate | 1711 | C12H14O3 | 206 |
| 232 | (Z)-Ligustilide | 1732 | C12H14O2 | 190 |
| 233 | Heyderiol | 2374 | C22H30O4 | 358 |
| 234 | (E)-Ligustilide | 1782 | C12H14O2 | 190 |
| 235 | 7,11-Dimethylheptadecane | 1792 | C19H40 | 268 |
| 236 | Avocadynofuran | 1796 | C17H26O | 246 |
| 237 | Galaxolide | 1838 | C18H26O | 258 |
| 238 | Traseolide | 1840 | C18H26O | 258 |
| 239 | Tonalide | 1850 | C18H26O | 258 |
| 240 | 1-Nonadecene | 1875 | C19H38 | 266 |
| 241 | Nonadecane | 1900 | C19H40 | 268 |
| 242 | Falcarinol | 2028 | C17H24O | 244 |
| 243 | Trichocoleine | 1875 | C14H18O4 | 250 |
| 244 | Ambrettolide | 1905 | C16H28O2 | 252 |
| 245 | Methyl 4-Hydroxy-3-methoxy-5- | 1833 | C14H18O4 | 250 |
| | (1,1-dimethylprop-2-enyl)-benzoate | | | |
| 246 | (E)-Benzyl cinnamate | 2023 | C16H14O2 | 238 |
| 247 | trans-Pinocarvyl formate | 1228 | C11H16O2 | 180 |
| 248 | Hex-5-en-1-ol | 820 | C6H12O | 100 |
| 249 | Hex-5-en-3-ol | 832 | C6H12O | 100 |
| 250 | 1-Hexanol | 837 | C6H14O | 102 |
| 251 | (Z)-Hex-3-en-1-ol | 851 | C6H12O | 100 |
| 252 | (E)-Hex-3-en-1-ol | 851 | C6H12O | 100 |
| 253 | (Z)-Hex-2-en-1-ol | 861 | C6H12O | 100 |
| 254 | 2-Heptanone | 871 | C7H14O | 114 |
| 255 | 2-Heptanol | 880 | C7H16O | 116 |
| 256 | 3-Heptanol | 877 | C7H16O | 116 |
| 257 | n-Heptanal | 882 | C7H14O | 114 |
| 258 | Santene | 884 | C9H14 | 122 |
| 259 | 2-Methyl-1-hexanol | 917 | C7H16O | 116 |
| 260 | Tricyclene | 927 | C10H16 | 136 |
| 261 | a-Pinene | 936 | C10H16 | 136 |
| 262 | Benzaldehyde | 941 | C7H6O | 106 |
| 263 | a-Fenchene | 941 | C10H16 | 136 |
| 264 | Thuja-2,4(10)-diene | 946 | C10H14 | 134 |
| 265 | 6-Methyl-2-heptanol | 950 | C8H18O | 130 |
| 266 | Camphene | 950 | C10H16 | 136 |
| 267 | 1-Octen-3-ol | 962 | C8H16O | 128 |
| 268 | 3-Octanone | 969 | C8H16O | 128 |
| 269 | 4-Octanol | 973 | C8H18O | 130 |
| 270 | 2-Octanol | 981 | C8H18O | 130 |
| 271 | 3-Octanol | 981 | C8H18O | 130 |
| 272 | 2-Pentylfuran | 981 | C9H14O | 138 |
| 273 | Yomogialcohol | 991 | C10H18O | 154 |
| 274 | 3,6-Dimethyl-3-heptanol | 990 | C9H20O | 144 |
| 275 | D 2-Carene | 1000 | C10H16 | 136 |
| 276 | a-Phellandrene | 1002 | C10H16 | 136 |
| 277 | (Z)-Hex-3-enyl acetate | 1002 | C8H14O2 | 142 |
| 278 | p-Methylanisol | 1004 | C8H10O | 122 |
| 279 | Benzyl alcohol | 1006 | C7H8O | 108 |
| 280 | D 3-Carene | 1010 | C10H16 | 136 |
| 281 | Phenylacetaldehyde | 1012 | C8H8O | 120 |
| 282 | m-Cymene | 1013 | C10H14 | 134 |
| 283 | p-Cymene | 1015 | C10H14 | 134 |
| 284 | Salicylaldehyde | 1020 | C7H6O2 | 122 |
| 285 | Limonene | 1025 | C10H16 | 136 |
| 286 | 1,8-Cineol | 1024 | C10H18O | 154 |
| 287 | (Z)-b-Ocimene | 1029 | C10H16 | 136 |
| 288 | (E)-2-Octenal | 1034 | C8H14O | 126 |
| 289 | 5,5-Dimethylbut-3-enolide | 916 | C6H8O2 | 112 |
| 290 | Methyl 3-methylfuroate | 1038 | C7H8O3 | 140 |
| 291 | (E)-b-Ocimene | 1041 | C10H16 | 136 |
| 292 | Oct-3-en-1-ol (Isomer 1) | 1044 | C8H16O | 128 |
| 293 | Artemisia ketone | 1044 | C10H16O | 152 |
| 294 | cis-Dihydroroseoxide | 1047 | C10H20O | 156 |
| 295 | d-Terpineol | 1155 | C10H16O | 152 |
| 296 | g-Terpinene | 1051 | C10H16 | 136 |
| 297 | trans-Sabinene hydrate | 1053 | C10H18O | 154 |
| 298 | Dihydromyrcenol | 1058 | C10H20O | 156 |
| 299 | Non-1-en-3-ol | 1058 | C9H18O | 142 |
| 300 | trans-Linalooloxide (furanoid) | 1058 | C10H20O2 | 172 |
| 301 | p-Mentha-3,8-diene | 1059 | C10H16 | 136 |
| 302 | Benzyl formate | 1060 | C8H8O2 | 136 |
| 303 | m-Cresol | 1061 | C7H8O | 108 |
| 304 | p-Cresol | 1062 | C7H8O | 108 |
| 305 | 1-Octanol | 1063 | C8H18O | 130 |
| 306 | Fenchone | 1069 | C10H16O | 152 |
| 307 | o-Guiacol | 1072 | C7H8O2 | 124 |
| 308 | Methyl benzoate | 1072 | C8H8O2 | 136 |
| 309 | cis-Linalool oxide (furanoid) | 1072 | C10H18O2 | 170 |
| 310 | Artemisia alcohol | 1073 | C10H18O | 154 |
| 311 | Dehydrolinalool | 1073 | C10H16O | 152 |
| 312 | trans-Dihydroroseoxide | 1075 | C10H20O | 156 |
| 313 | 4-Nonanol | 1076 | C9H20O | 144 |
| 314 | Terpinolene | 1082 | C10H16 | 136 |
| 315 | cis-Sabinene hydrate | 1082 | C10H18O | 154 |
| 316 | 2-Nonanol | 1085 | C9H20O | 144 |
| 317 | Linalool | 1086 | C10H18O | 154 |
| 318 | Photocitral B | 1086 | C10H16O | 152 |
| 319 | a-Thujone | 1089 | C10H16O | 152 |
| 320 | 2,2′,5,6-Tetramethyl cyclohexanone | 1092 | C10H18O | 154 |
| | (Isomer 1) | | | |
| 321 | 1-Oct-3-enyl acetate | 1093 | C10H18O2 | 170 |
| 322 | 4,8-Dimethyl-1,3,7-nonatriene | 1096 | C11H18 | 150 |
| | (Isomer 1) | | | |
| 323 | a-Pinene epoxide (Isomer 1) | 1096 | C10H16O | 152 |
| 324 | a-Fenchol | 1099 | C10H18O | 154 |
| 325 | cis-Rose oxide | 1100 | C10H18O | 154 |
| 326 | Isochrysanthenone | 1086 | C10H14O | 150 |
| 327 | b-Thujone | 1103 | C10H16O | 152 |
| 328 | a-Campholenal | 1105 | C10H16O | 152 |
| 329 | 2,2′,5,6-Tetramethyl cyclohexanone | 1106 | C10H18O | 154 |
| | (Isomer 2) | | | |
| 330 | 2-Methyl-5-propionylfuran | 1108 | C8H10O2 | 138 |
| 331 | cis-p-Menth-2-en-1-ol | 1108 | C10H18O | 154 |
| 332 | (Z)-Ocimenoxide | 1115 | C10H16O | 152 |
| 333 | 4,8-Dimethylnona-1,3,7-triene | 1115 | C11H18 | 150 |
| | (Isomer 2) | | | |
| 334 | a-Pinene epoxide (Isomer 2) | 1116 | C10H16O | 152 |
| 335 | trans-Rose oxide | 1116 | C10H18O | 154 |
| 336 | Dihydrolinalool | 1118 | C10H20O | 156 |
| 337 | Ipsdienol | 1123 | C10H16O | 152 |
| 338 | Camphor | 1123 | C10H16O | 152 |
| 339 | trans-p-Menth-2-en-1-ol | 1123 | C10H18O | 154 |
| 340 | (E)-Ocimenoxide | 1125 | C10H16O | 152 |
| 341 | p-Mentha-1,5-diene-8-ol | 1127 | C10H16O | 152 |
| 342 | trans-Pinocarveol | 1126 | C10H16O | 152 |
| 343 | cis-Limonene oxide | 1126 | C10H16O | 152 |
| 344 | Photocitral A | 1127 | C10H16O | 152 |
| 345 | o-Cymenene | 1076 | C10H12 | 132 |
| 346 | (E)-Tagetone | 1128 | C10H16O | 152 |
| 347 | (Z)-Tagetone | 1136 | C10H16O | 152 |
| 348 | trans-Limonene oxide | 1130 | C10H16O | 152 |
| 349 | Isopulegol | 1132 | C10H18O | 154 |
| 350 | 1,3-Dimethoxybenzene | 1136 | C8H10O2 | 138 |
| 351 | Menthone | 1136 | C10H18O | 154 |
| 352 | Pinocarvone | 1137 | C10H14O | 150 |
| 353 | b-Terpineol | 1137 | C10H18O | 154 |
| 354 | (E)-Non-2-enal | 1139 | C9H16O | 140 |
| 355 | Isoneral | 1140 | C10H16O | 152 |
| 356 | cis-b-Terpineol | 1141 | C10H18O | 154 |
| 357 | Isoborneol | 1142 | C10H18O | 154 |
| 358 | Karahanaenone | 1142 | C10H16O | 152 |
| 359 | cis- or trans-Linalool oxide | 1144 | C10H18O2 | 170 |
| | (pyranoid) | | | |
| 360 | Isomenthone | 1146 | C10H18O | 154 |
| 361 | cis-Chrysanthenol | 1147 | C10H16O | 152 |
| 362 | 2-Hydroxyethyl-4-methylbenzene | 1147 | C9H12O | 136 |
| 363 | 4-Isopropylcyclohexanone | 1148 | C9H16O | 140 |
| 364 | cis- or trans-Linalool oxide | 1148 | C10H18O2 | 170 |
| | (pyranoid) | | | |
| 365 | cis-Isopulegone | 1148 | C10H16O | 152 |
| 366 | Methyl phenyl acetate | 1148 | C9H10O2 | 150 |
| 367 | cis-Thujol | 1149 | C10H18O | 154 |
| 368 | b-Pinene epoxide | 1149 | C10H16O | 152 |
| 369 | Ethyl benzoate | 1150 | C9H10O2 | 150 |
| 370 | Borneol | 1150 | C10H18O | 154 |
| 371 | Lavandulol | 1150 | C10H18O | 154 |
| 372 | Umbellulone | 1152 | C10H14O | 150 |
| 373 | trans-Chrysanthemol | 1153 | C10H18O | 154 |
| 374 | Neomenthol | 1156 | C10H20O | 156 |
| 375 | Viridene | 1159 | C10H12O | 148 |
| 376 | Isogeranial | 1156 | C10H16O | 152 |
| 377 | cis-Chrysanthemol | 1157 | C10H18O | 154 |
| 378 | Benzoic acid | 1160 | C7H6O2 | 122 |
| 379 | 3-Thujene-10-al | 1158 | C10H14O | 150 |
| 380 | Cryptone | 1160 | C9H14O | 138 |
| 381 | Terpinen-4-ol | 1164 | C10H18O | 154 |
| 382 | b-Pinene epoxide (Isomer) | 1170 | C10H16O | 152 |
| 383 | Nona-2,4-dienal | 1170 | C9H14O | 138 |
| 384 | Methyl salicylate | 1171 | C8H8O3 | 152 |
| 385 | 2-Methyl-2-borneol | 1175 | C11H20O | 168 |
| 386 | 2-Allylphenol | 1174 | C9H10O | 134 |
| 387 | Thujopsa-3-one | 1645 | C15H24O | 220 |
| 388 | 7-Hydroxyhotrienol | 1177 | C10H18O2 | 170 |
| 389 | Myrtenol | 1178 | C10H16O | 152 |
| 390 | cis-Piperitol | 1181 | C10H18O | 154 |
| 391 | Safranal | 1182 | C10H14O | 150 |
| 392 | Estragol (Methylchavicol) | 1175 | C10H12O | 148 |
| 193 | 2-Decanol | 1188 | C10H22O | 158 |
| 394 | g-Terpineol | 1188 | C10H18O | 154 |
| 195 | (E,E)-Nona-2,4-dienal | 1188 | C9H14O | 138 |
| 396 | Methyl a-cyclogeranate | 1190 | C11H18O2 | 182 |
| 197 | trans-Piperitol | 1193 | C10H18O | 154 |
| 398 | Chrysanthenone | 1110 | C10H14O | 150 |
| 399 | Fenchyl acetate | 1205 | C12H20O2 | 196 |
| 400 | Benzylacetone | 1207 | C10H12O | 148 |
| 401 | 2-epi-Thujopsa-3-one | 1634 | C15H24O | 220 |
| 402 | Carvotanacetone | 1220 | C10H16O | 152 |
| 403 | Menthol | 1172 | C10H20O | 156 |
| 404 | Isomenthol | 1176 | C10H20O | 156 |
| 405 | a-Terpinyl acetate | 1335 | C10H16 | 136 |
| 406 | Octyl acetate | 1188 | C10H20O2 | 172 |
| 407 | Dillether | 1170 | C10H16O | 152 |
| 408 | (E)-Ethyl cinnamate | 1439 | C11H12O2 | 176 |
| 409 | b-Ionone | 1468 | C13H20O | 192 |
| 410 | Piperonal | 1294 | C8H6O3 | 150 |
| 411 | Vanilline | 1355 | C8H8O3 | 152 |
| 412 | Coumarin | 1392 | C9H6O2 | 146 |
| 413 | (Z)-2-Hexylcinnamic aldehyde | 1725 | C15H20O | 216 |
| 414 | 1-Phenylethyl acetate | 1166 | C10H12O2 | 164 |
| 415 | (Z)-2-Pentylcinnamaldehyde | 1632 | C14H18O | 202 |
| 416 | Benzyl salicylate | 1847 | C14H12O3 | 228 |
| 417 | Menthofuran | 1150 | C10H14O | 150 |
| 418 | a-Campholenol | 1190 | C10H18O | 154 |
| 419 | Methyl jasmonate | 1611 | C13H20O3 | 224 |
| 420 | Isophytol | 1949 | C20H40O | 296 |
| 421 | Phytol | 2114 | C20H40O | 296 |
| 422 | (E)-Anyl 2-methylbutyrate | 1651 | C14H18O2 | 218 |
| 423 | (E)-4-4Propenylphenol tiglate | 1765 | C14H16O2 | 216 |
| 424 | trans-Epoxypseudoisoeugenyl-2- | 1871 | C15H20O4 | 264 |
| | methylbutyrate | | | |
| 425 | (E)-Pseudoisoeugenyl tiglate | 1895 | C15H18O3 | 246 |
| 426 | trans-Epoxypseudoisoeugenol tiglate | 1942 | C15H18O4 | 262 |
| 427 | Dictyotene | 1155 | C11H18 | 150 |
| 428 | Desmarestene | 1168 | C11H14 | 146 |
| 429 | Dictyopterene A | 1099 | C11H18 | 150 |
| 430 | Ectocarpene | 1136 | C11H16 | 148 |
| 431 | (E)-Ectocarpene | 1147 | C11H16 | 148 |
| 432 | cis-Hormosirene | 1152 | C11H16 | 148 |
| 433 | trans-Hormosirene | 1160 | C11H16 | 148 |
| 434 | (Z)-Multifidene | 1040 | C11H16 | 148 |
| 435 | (E)-Multifidene | 1047 | C11H16 | 148 |
| 436 | (E)-Aucantene | 1062 | C11H16 | 148 |
| 437 | (E)-Aucantene | 1077 | C11H16 | 148 |
| 438 | cis-Dihydromultifidene | 1052 | C11H18 | 150 |
| 439 | trans-Dihydromultifidene | 1058 | C11H18 | 150 |
| 440 | Neothujol | 1136 | C10H16O | 152 |
| 441 | (Z)-Methyl cinnamate | 1270 | C10H10O2 | 162 |
| 442 | Isothujol | 1121 | C10H16O | 152 |
| 443 | Neoisothujol | 1132 | C10H16O | 152 |
| 444 | Citronellal | 1129 | C10H18O | 154 |
| 445 | 2-Methyl-2-pentanol | 944 | C6H14O | 102 |
| 446 | 6-Acetoxy-p-mentha-1(7),8-diene | 1312 | C12H18O2 | 194 |
| | (Isomer 1) | | | |
| 447 | n-Nonanal | 1076 | C9H18O | 142 |
| 448 | 5,7-Dimethylocta-1,6-diene | 911 | C10H18 | 138 |
| 449 | Dec-9-en-1-ol | 1240 | C10H20O | 156 |
| 450 | Elsholtzia ketone | 1175 | C10H14O2 | 166 |
| 451 | a-Dehydroelsholtzia ketone | 1188 | C10H12O2 | 164 |
| 452 | Dehydroelsholtzia ketone | 1277 | C10H12O2 | 164 |
| 453 | 4-Methyl-3-heptanol | 956 | C8H18O | 130 |
| 454 | 6-Methylhept-5-en-2-ol (Sulcatol) | 981 | C8H16O | 128 |
| 455 | b-Helmiscapene | 1446 | C15H24 | 204 |
| 456 | 2,2-Dimethyl-4-oxocyclohexane-1- | 1132 | C9H14O2 | 154 |
| | carbaldehyde | | | |
| 457 | Menth-1-en-9-ol | 1283 | C10H18O | 154 |
| 458 | cis-Dihydrocarvone | 1172 | C10H16O | 152 |
| 459 | trans-Dihydrocarvone | 1177 | C10H16O | 152 |
| 460 | Limonen-10-ol | 1272 | C10H16O | 152 |
| 461 | Tuberolactone | 1437 | C10H14O2 | 166 |
| 462 | trans-Carveol | 1200 | C10H16O | 152 |
| 463 | Dihydrocarveol (Isomer 1) | 1176 | C10H18O | 154 |
| 464 | Dihydrocarveol (Isomer 2) | 1193 | C10H18O | 154 |
| 465 | Dihydrocarveol (Isomer 3) | 1205 | C10H18O | 154 |
| 466 | cis-Carveol | 1210 | C10H16O | 152 |
| 467 | 4-Methoxyphenylacetone | 1343 | C10H12O2 | 164 |
| 468 | 4-Methoxypropiophenone | 1415 | C10H12O2 | 164 |
| 469 | Grandisol | 1200 | C10H18O | 154 |
| 470 | Hotrienol | 1083 | C10H16O | 152 |
| 471 | Isopinocampheol | 1168 | C10H16O | 152 |
| 472 | (E)-Pseudoisoeugenyl-2-methyl | 1823 | C15H20O3 | 248 |
| | butyrate | | | |
| 473 | Falcarinone | 1990 | C17H22O | 242 |
| 474 | Ethyl salicylate | 1245 | C9H10O3 | 166 |
| 475 | 1,2-Dihydro-1,1,6-trimethyl- | 1339 | C13H16 | 172 |
| | naphthalene | | | |
| 476 | g-Hexanolide | 1006 | C6H10O2 | 114 |
| 477 | g-Heptanolide | 1103 | C7H12O2 | 128 |
| 478 | g-Octanolide | 1208 | C8H14O2 | 142 |
| 479 | g-Nonanolide | 1318 | C9H16O2 | 156 |
| 480 | g-Decanolide | 1433 | C10H18O2 | 170 |
| 481 | g-Undecanolide | 1547 | C11H20O2 | 184 |
| 482 | g-Dodecanolide | 1656 | C12H22O2 | 198 |
| 483 | g-Tetradecanolide | 1866 | C14H26O2 | 226 |
| 484 | d-Nonanolide | 1348 | C9H16O2 | 156 |
| 485 | d-Octanolide | 1240 | C8H14O2 | 142 |
| 486 | d-Heptanolide | 1156 | C7H12O2 | 128 |
| 487 | d-Decanolide | 1461 | C10H18O2 | 170 |
| 488 | (E)-a-Damascone | 1375 | C13H20O | 192 |
| 489 | 4-Methyl-3-heptanone | 918 | C8H16O | 128 |
| 490 | a-Ionone | 1409 | C13H20O | 192 |
| 491 | p-Methylacetophenone | 1156 | C9H10O | 134 |
| 492 | b-Cyclocitral | 1195 | C10H16O | 152 |
| 493 | cis-a-Irone | 1520 | C14H22O | 206 |
| 494 | cis-g-Irone | 1525 | C14H22O | 206 |
| 495 | Dodecanal | 1389 | C12H24O | 184 |
| 496 | Methyl linolenate | 2102 | C19H32O2 | 292 |
| 497 | Geranylacetone | 1430 | C13H22O | 194 |
| 498 | trans-Isolimonene | 975 | C10H16 | 136 |
| 499 | cis-Myrtanol | 1238 | C10H18O | 154 |
| 500 | n-Octanal | 981 | C8H16O | 128 |
| 501 | p-Menth-1-ene | 1017 | C15H18 | 198 |
| 502 | (E)-Jasmone | 1356 | C11H16O | 164 |
| 503 | trans-Myrtanol | 1240 | C10H18O | 154 |
| 504 | allo-Ocimene | 1113 | C10H16 | 136 |
| 505 | (4E,6Z)-allo-Ocimene | 1126 | C10H16 | 136 |
| 506 | Dodecanol | 1472 | C12H26O | 186 |
| 507 | 6-Acetoxy-p-menta-1,8-diene | 1341 | C12H18O2 | 194 |
| 508 | b-Citronellene | 943 | C15H18 | 198 |
| 509 | n-Nonanol | 1149 | C9H20O | 144 |
| 510 | trans-Sabinol | 1120 | C10H16O | 152 |
| 511 | 3,5-Dimethoxytoluene | 1231 | C9H12O2 | 152 |
| 512 | Phantolide | 1712 | C17H24O | 244 |
| 513 | Perilla alcohol | 1280 | C10H16O | 152 |
| 514 | b-Phellandrene | 1023 | C10H16 | 136 |
| 515 | b-Phenylethanol | 1085 | C8H10O | 122 |
| 516 | Citronellol | 1213 | C10H20O | 156 |
| 517 | Methyleugenol | 1369 | C11H14O2 | 178 |
| 518 | 2-Nonanone | 1074 | C9H18O | 142 |
| 519 | 2-Decanone | 1176 | C10H20O | 156 |
| 520 | 2-Dodecanone | 1381 | C12H24O | 184 |
| 521 | 4-Isopropylcyclohexanol (Isomer 2) | 1130 | C9H18O | 142 |
| 522 | Moskachane B | 1794 | C13H16O3 | 220 |
| 523 | Moskachane D | 2001 | C15H20O3 | 248 |
| 524 | cis-Verbenol | 1132 | C10H16O | 152 |
| 525 | (Z)-Salvene | 849 | C9H16 | 124 |
| 526 | (E)-Salvene | 859 | C9H16 | 124 |
| 527 | Santolinatriene | 909 | C10H16 | 136 |
| 528 | a-Thujene | 932 | C10H16 | 136 |
| 529 | Sabinene | 973 | C10H16 | 136 |
| 530 | a-Terpinene | 1013 | C10H16 | 136 |
| 531 | trans-Verbenol | 1136 | C10H14O | 150 |
| 532 | Verbenone | 1183 | C10H14O | 150 |
| 533 | Z-Cinnamaldehyde | 1185 | C9H8O | 132 |
| 534 | 3-Phenylpropanol | 1201 | C9H12O | 136 |
| 535 | (E)-Cinnamyl alcohol | 1275 | C9H10O | 134 |
| 536 | b-Irone | 1566 | C14H22O | 206 |
| 537 | Benzyl acetate | 1134 | C9H10O2 | 150 |
| 538 | Indole | 1257 | C8H7N | 117 |
| 539 | d-Jasmolactone | 1450 | C10H16O2 | 168 |
| 540 | N-Acetyl methyl anthranilate | 1565 | C10H11O3N | 193 |
| 541 | Benzyl benzoate | 1730 | C14H12O2 | 212 |
| 542 | 6-Methylhept-5-en-2-one | 978 | C8H14O | 126 |
| 543 | Rosefuran | 1091 | C10H14O | 150 |
| 544 | Rosefuran epoxide | 1161 | C10H14O2 | 166 |
| 545 | b-Pinene | 978 | C10H16 | 136 |
| 546 | Myrcene | 987 | C10H16 | 136 |
| 547 | Oct-3-en-1-ol (Isomer 2) | 1049 | C8H16O | 128 |
| 548 | 6-Acetoxy-p-mentha-1(7),8-diene | 1343 | C12H18O2 | 194 |
| | (Isomer 2) | | | |
| 549 | (E)-4-Propenylphenol angelate | 1751 | C14H16O2 | 216 |
| 550 | cis-Epoxypseudoisoeugenyl-2- | 1870 | C15H20O4 | 264 |
| | methyl butyrate | | | |
| 551 | Myrtenal | 1172 | C10H14O | 150 |
| 552 | (E)-Ocimenone | 1219 | C10H14O | 150 |
| 553 | (Z)-Ocimenone | 1209 | C10H14O | 150 |
| 554 | Isomenthyl acetate | 1298 | C12H22O2 | 198 |
| 555 | Thymoquinone | 1215 | C10H12O2 | 164 |
| 556 | Cymen-9-ol | 1157 | C10H14O | 150 |
| 557 | 8,9-Dehydrothymol | 1190 | C10H12O | 148 |
| 558 | (Z)-Methyl p-hydroxycinnamate | 1603 | C10H10O3 | 178 |
| 559 | b-Thujaplicine | 1449 | C10H12O2 | 164 |
| 560 | n-Undecane | 1100 | C11H24 | 156 |
| 561 | n-Nonane | 906 | C9H20 | 128 |
| 562 | Pinocamphone | 1139 | C10H16O | 152 |
| 563 | Isopinocamphone | 1151 | C10H16O | 152 |
| 564 | Methyl 2-methylbutyrate | 954 | C6H12O2 | 116 |
| 565 | 6-Methylhept-5-enal | 985 | C8H14O | 126 |
| 566 | Furomyrcenol | 1256 | C10H14O2 | 166 |
| 567 | a-Ionone epoxide (Isomer 1) | 1516 | C13H20O2 | 208 |
| 568 | o-Cresol | 1037 | C7H8O | 108 |
| 569 | (E)-2-Hexenal | 832 | C6H10O | 98 |
| 570 | Ethyl 2-methylbutyrate | 843 | C7H14O2 | 130 |
| 571 | p-Mentha-2,4(8)-diene | 1077 | C10H16 | 136 |
| 572 | p-Mentha-1,3,8-triene | 1101 | C10H16 | 136 |
| 573 | Neroloxide | 1137 | C10H16O | 152 |
| 574 | Neoisopulegol | 1150 | C10H18O | 154 |
| 575 | (E,E)-Nona-3,6-dien-1-ol | 1145 | C9H16O | 140 |
| 576 | n-Pentylbenzene | 1150 | C11H16 | 148 |
| 577 | (Z)-Ethyl oct-5-enoate | 1174 | C10H18O2 | 170 |
| 578 | a-Terpineol | 1176 | C10H18O | 154 |
| 579 | 2a-Hydroxy-1,8-cineol | 1196 | C10H18O2 | 170 |
| 580 | cis-Pulegol | 1215 | C10H18O | 154 |
| 581 | 3b-Hydroxy-1,8-cineol | 1229 | C10H18O2 | 170 |
| 582 | Linalyl acetate | 1239 | C12H20O2 | 196 |
| 583 | Isopiperitenone | 1240 | C10H14O | 150 |
| 584 | Piperitenone | 1318 | C10H14O | 150 |
| 585 | Piperitenone oxide | 1335 | C10H14O2 | 166 |
| 586 | Geranyl acetate | 1362 | C12H20O2 | 196 |
| 587 | 2-Methylbutyl benzoate | 1419 | C12H16O2 | 192 |
| 588 | Myristicine | 1489 | C11H12O3 | 192 |
| 589 | Acetophenone | 1036 | C8H8O | 120 |
| 590 | Dihydrotagetone | 1047 | C10H18O | 154 |
| 591 | p-Cymenene | 1075 | C10H12 | 132 |
| 592 | Piperiton epoxid | 1232 | C10H16O2 | 168 |
| 593 | Nepetalacton (Isomer 2) | 1360 | C10H14O2 | 166 |
| 594 | 3,4-Dimethyl-5-pentyl-5H-furan-2- | 1481 | C11H18O2 | 182 |
| | one | | | |
| 595 | Methyl p-methoxybenzoate | 1338 | C9H10O3 | 166 |
| 596 | (E)-o-Methoxycinnamyl alcohol | 1488 | C10H12O2 | 164 |
| 597 | (E)-m-Methoxycinnamyl alcohol | 1511 | C10H12O2 | 164 |
| 598 | (E)-p-Methoxycinnamyl alcohol | 1523 | C10H12O2 | 164 |
| 599 | Perillene | 1090 | C10H14O | 150 |
| 600 | Methyl anthranilate | 1308 | C8H9O2N | 151 |
| 601 | 1-(3-Methoxyphenyl)-2- | 1735 | C15H16O | 212 |
| | phenylethane | | | |
| 602 | 1-Phenyl-2-(3,5-dimethoxyphenyl)- | 1962 | C16H18O2 | 242 |
| | ethane | | | |
| 603 | 1-(3-Methoxyphenyl)-2-(4- | 1988 | C16H18O2 | 242 |
| | methoxyphenyl)-ethane | | | |
| 604 | Neryl isobutyrate | 1468 | C14H24O2 | 224 |
| 605 | Zingiberenol | 1596 | C14H24O | 208 |
| 606 | Encecalin | 1813 | C14H16O3 | 232 |
| 607 | Ethyl p-methoxybenzoate | 1415 | C10H12O3 | 180 |
| 608 | Albanone | 1389 | C12H18O | 178 |
| 609 | 7,10-Anhydro-11,12- | 1449 | C15H24O | 220 |
| | dihydrochiloscypholone | | | |
| 610 | 1(11)-Africanen-8-ol | 1486 | C15H24O | 220 |
| 611 | Atractylone | 1497 | C15H20O | 216 |
| 612 | Conocephalenol | 1497 | C15H26O | 222 |
| 613 | Cubebol | 1514 | C15H26O | 222 |
| 614 | Photosantalol | 1511 | C15H24O | 220 |
| 615 | Cyperene epoxide | 1524 | C15H24O | 220 |
| 616 | Isoafricanol | 1529 | C15H26O | 222 |
| 617 | cis-Cadina-4,6-dien-11-ol | 1531 | C15H24O | 220 |
| 618 | Elema-1,3-dien-7-ol | 1531 | C15H24O | 220 |
| 619 | Tamariscol | 1535 | C15H26O | 222 |
| 620 | Pacifigorgiol | 1539 | C15H26O | 222 |
| 621 | (E,E)-Methyl 10-oxofarnesoate | 1896 | C16H26O3 | 266 |
| 622 | b-Caryophyllene oxide | 1546 | C15H24O | 220 |
| 623 | Africanone | 1547 | C15H22O | 218 |
| 624 | 1,8-Oxidocadin-4-ene | 1551 | C15H24O | 220 |
| 625 | 4bH,5aH-cis-Eudesm-6-en-11-ol | 1555 | C15H26O | 222 |
| 626 | Dactylol | 1556 | C15H26O | 222 |
| 627 | cis-Sesquisabinenhydrate | 1558 | C15H26O | 222 |
| 628 | 11,12-Dihydrochiloscyphone | 1558 | C15H24O | 220 |
| 629 | Aromadendran-5-ol | 1562 | C15H26O | 222 |
| 630 | Oxidohimachalene | 1557 | C15H22O | 218 |
| 631 | (+)-Marsupellol | 1564 | C15H24O | 220 |
| 632 | b-Himachalol | 1638 | C15H26O | 222 |
| 633 | Maaliol | 1565 | C15H26O | 222 |
| 634 | Deoxopinguisone | 1563 | C15H22O | 218 |
| 635 | Palustrol | 1569 | C15H26O | 222 |
| 636 | 4a-Hydroxygermacra-1(10),5-diene | 1571 | C15H26O | 222 |
| 637 | Spathulenol | 1572 | C15H24O | 220 |
| 638 | 4-Dehydroviridiflorol | 1572 | C15H24O | 220 |
| 639 | Caryophyllene oxide | 1578 | C15H24O | 220 |
| 640 | 7-Acetoxyelema-1,3,8-triene | 1584 | C17H26O2 | 262 |
| 641 | Globulol | 1589 | C15H26O | 222 |
| 642 | Cubeban-11-ol | 1591 | C15H26O | 222 |
| 643 | Salvial-4(14)-en-1-one | 1592 | C15H24O | 220 |
| 644 | Bisabola-2,10-diene 1,9-oxide | 1592 | C15H24O | 220 |
| 645 | b-Oplopenone | 1595 | C15H24O | 220 |
| 646 | Longiborneol | 1597 | C15H26O | 222 |
| 647 | Rosifoliol | 1599 | C15H26O | 222 |
| 648 | Ledol | 1600 | C15H26O | 222 |
| 649 | 2-Methyl-1-(octahydro-7,7a- | 1601 | C15H26O | 222 |
| | dimethyl-1H-inden-1-yl)-propan-1- | | | |
| | one | | | |
| 650 | Eudesm-4-en-7-ol | 1604 | C15H26O | 222 |
| 651 | Rearrangement product from | 1608 | C15H22O | 218 |
| | Grimaldone | | | |
| 652 | Maalian-5-ol | 1607 | C15H26O | 222 |
| 653 | 10-epi-g-Eudesmol | 1609 | C15H26O | 222 |
| 654 | ar-Curcumen-7-ol | 1610 | C15H22O | 218 |
| 655 | Amorpha-4,7-dien-11-ol | 1610 | C15H24O | 220 |
| 656 | 5-Guaiene-11-ol | 1619 | C15H26O | 222 |
| 657 | g-Eudesmol | 1618 | C15H26O | 222 |
| 658 | Alismol | 1619 | C15H24O | 220 |
| 659 | Gymnomitrone | 1620 | C15H22O | 218 |
| 660 | Isospathulenol | 1625 | C15H24O | 220 |
| 661 | Isogymnomitrol | 1625 | C15H24O | 220 |
| 662 | Furanoeudesm-1,3-diene | 1630 | C15H18O | 214 |
| 663 | Amorpha-4-en-7-ol | 1629 | C15H26O | 222 |
| 664 | Eudesm-3,11-dien-5-ol | 1632 | C15H24O | 220 |
| 665 | Hinesol | 1632 | C15H26O | 222 |
| 666 | T-Muurolol | 1633 | C15H26O | 222 |
| 667 | (E,E)-Germacradiene-11-ol | 1633 | C15H26O | 222 |
| 668 | T-Cadinol | 1633 | C15H26O | 222 |
| 669 | Gymnomitr-3(15)-en-4-one | 1635 | C15H22O | 218 |
| 670 | 1(10)-Spirovetivene-7b-ol | 1636 | C15H26O | 222 |
| 671 | Muurola-3,7(11)-dien-1-ol | 1637 | C15H24O | 220 |
| 672 | 6-Himachalen-9b-ol | 1638 | C15H26O | 222 |
| 673 | Gymnomitran-4-one | 1639 | C15H24O | 220 |
| 674 | b-Eudesmol | 1641 | C15H26O | 222 |
| 675 | Furanoeremophilene | 1642 | C15H22O | 218 |
| 676 | 2-Himachalen-7b-ol | 1642 | C15H26O | 222 |
| 677 | a-Cadinol | 1643 | C15H26O | 222 |
| 678 | Eudesm-4(15)-en-7-ol | 1643 | C15H24O | 220 |
| 679 | 2-Methyl-1-(octahydro-7,7a- | 1645 | C15H28O | 224 |
| | dimethyl-1H-inden-1-yl)-propan-1- | | | |
| | ol | | | |
| 680 | Eudesm-11-en-4a-ol | 1649 | C15H26O | 222 |
| 681 | 1(10)-Valencen-7b-ol | 1646 | C15H26O | 222 |
| 682 | Valerianol | 1647 | C15H26O | 222 |
| 683 | Eudesm-3-en-7-ol | 1650 | C15H26O | 222 |
| 684 | 10-epi-trans-Dracunculifoliol | 1591 | C15H26O | 222 |
| 685 | 7-epi-a-Eudesmol | 1653 | C15H26O | 222 |
| 686 | Acorenol B | 1654 | C15H26O | 222 |
| 687 | Bisabolol oxide B | 1654 | C15H26O2 | 238 |
| 688 | Aromadendran-12-ol | 1654 | C15H24O | 220 |
| 689 | Grimaldone | 1656 | C15H22O | 218 |
| 690 | Gymnomitrol | 1657 | C15H24O | 220 |
| 691 | Eudesm-4(15)-en-6-ol | 1656 | C15H26O | 222 |
| 692 | Saccogynol | 1660 | C15H22O | 218 |
| 693 | Valeranone | 1664 | C15H26O | 222 |
| 694 | 4-epi-Acorenone | 1664 | C15H24O | 220 |
| 695 | Gymnomitr-3(15)-en-4a-ol | 1665 | C15H24O | 220 |
| 696 | Acorenol | 1667 | C15H26O | 222 |
| 697 | epi-Cyclosantalal | 1668 | C15H24O | 220 |
| 698 | (Z)-g-Atlantone | 1669 | C15H22O | 218 |
| 699 | a-Alasken-6-ol | 1674 | C15H24O | 220 |
| 700 | Bisabolone oxide A | 1675 | C15H24O2 | 236 |
| 701 | Amorpha-4,7(11)-dien-8-one | 1679 | C15H22O | 218 |
| 702 | Amorpha-4,9-dien-2-ol | 1679 | C15H24O | 220 |
| 703 | Amorpha-4,9-dien-14-al | 1685 | C15H22O | 218 |
| 704 | Eudesm-3-en-6-ol | 1679 | C15H26O | 222 |
| 705 | Khusiol | 1680 | C15H26O | 222 |
| 706 | (E)-g-Atlantone | 1681 | C15H22O | 218 |
| 707 | Acorenone | 1681 | C15H24O | 220 |
| 708 | Cadina-1(10),4-dien-8a-ol | 1682 | C15H24O | 220 |
| 709 | Bicyclogermacren-14-al | 1684 | C15H22O | 218 |
| 710 | Cyperotundone | 1684 | C15H22O | 218 |
| 711 | (Z)-a-Atlantone | 1689 | C15H22O | 218 |
| 712 | Lanceol oxide | 1695 | C15H24O | 220 |
| 713 | Farnesol (Isomer 1) | 1694 | C15H26O | 222 |
| 714 | 6a-Hydroxygermacra-1(10),4-diene | 1687 | C15H26O | 222 |
| 715 | Acora-7(11),9-dien-2-one | 1706 | C15H22O | 218 |
| 716 | Valerenal | 1706 | C15H22O | 218 |
| 717 | a-Herbertenol | 1711 | C15H22O | 218 |
| 718 | Dihydrochiloscypholone | 1711 | C15H26O2 | 238 |
| 719 | Italicen-4-one | 1717 | C15H22O | 218 |
| 720 | Farnesol (Isomer 2) | 1718 | C15H26O | 222 |
| 721 | 10-epi-1,8-Oxidocadina-4-ene | 1539 | C15H24O | 220 |
| 722 | Neopetasone | 1733 | C15H22O | 218 |
| 723 | 7,14-Anhydroamorpha-4,9-diene | 1733 | C15H22O | 218 |
| 724 | Lepidozenal | 1744 | C15H22O | 218 |
| 725 | 7-Acetoxyelema-1,3-dien-8-ol | 1793 | C17H28O3 | 280 |
| 726 | Naviculol | 1734 | C15H26O | 222 |
| 727 | Bisabolol oxide A | 1740 | C15H26O2 | 238 |
| 728 | a-Cyperone | 1741 | C15H22O | 218 |
| 729 | Cyclocolorenone | 1745 | C15H22O | 218 |
| 730 | Gymnomitrol acetate | 1751 | C17H26O2 | 262 |
| 731 | b-Herbertenol | 1751 | C15H22O | 218 |
| 732 | (E)-a-Atlantone | 1754 | C15H22O | 218 |
| 733 | (Z)-Lanceol | 1755 | C15H24O | 220 |
| 734 | Cuparophenol | 1763 | C15H22O | 218 |
| 735 | Cedryl acetate | 1764 | C17H28O2 | 264 |
| 736 | 14-Oxocalamenene | 1768 | C15H20O | 216 |
| 737 | Isovalencenol | 1779 | C15H24O | 220 |
| 738 | Drimenol | 1750 | C15H26O | 222 |
| 739 | cis-5-Hydroxycalamenene | 1790 | C15H22O | 218 |
| 740 | Khusienol acetate | 1789 | C17H26O2 | 262 |
| 741 | Fukinanolide | 1798 | C15H22O2 | 234 |
| 742 | Bisabola-2,7(Z),10(Z)-triene-13-ol | 1806 | C15H24O | 220 |
| 743 | Cyperadione | 1820 | C15H24O2 | 236 |
| 744 | cis-Spiroether | 1850 | C13H12O2 | 200 |
| 745 | trans-Spiroether | 1853 | C13H12O2 | 200 |
| 746 | trans-4,8a-Dimethyl-4a,5- | 1350 | C12H20O | 180 |
| | epoxydecaline | | | |
| 747 | Peculiaroxide | 1416 | C15H26O | 222 |
| 748 | Furanoelemene | 1485 | C15H20O | 216 |
| 749 | Guaioxide | 1487 | C15H26O | 222 |
| 750 | Elemol | 1541 | C15H26O | 222 |
| 751 | Lemnalol | 1579 | C15H24O | 220 |
| 752 | Fokienol | 1582 | C15H24O | 220 |
| 753 | Thujopsane-2b-ol | 1593 | C15H26O | 222 |
| 754 | a-Alasken-8-ol | 1600 | C15H24O | 220 |
| 755 | 6-epi-Cubenol | 1602 | C15H26O | 222 |
| 756 | Widdrol | 1601 | C15H26O | 222 |
| 757 | Marsupellone | 1604 | C15H22O | 218 |
| 758 | Axinyssene | 1860 | C20H32 | 272 |
| 759 | Selina-1,3,7(11)-trien-8-one | 1616 | C15H20O | 216 |
| 760 | Myliol | 1617 | C15H22O | 218 |
| 761 | a-Acorenol | 1623 | C15H26O | 222 |
| 762 | Furanogermacrene | 1624 | C15H20O | 216 |
| 763 | Acora-3,7(11)-dien-6-ol | 1626 | C15H24O | 220 |
| 764 | b-Acorenol | 1626 | C15H26O | 222 |
| 765 | a-Alaskene-8-ol | 1632 | C15H24O | 220 |
| 766 | Microbiotol | 1632 | C15H26O | 222 |
| 767 | Isopinguisanine | 1638 | C15H20O2 | 232 |
| 768 | Gymnomitr-3(15)-en-4b-ol | 1653 | C15H24O | 220 |
| 769 | a-Eudesmol | 1653 | C15H26O | 222 |
| 770 | Isorotundenol | 1659 | C15H26O | 222 |
| 771 | Bulnesol | 1665 | C15H26O | 222 |
| 772 | Bicyclohumulenone | 1668 | C15H24O | 220 |
| 773 | Selina-4(15),11-dien-8-ol | 1670 | C15H24O | 220 |
| 774 | Smyrnicordifuran | 1673 | C15H18O2 | 230 |
| 775 | b-Sinensal | 1675 | C15H22O | 218 |
| 776 | Isocyperol | 1676 | C15H24O | 220 |
| 777 | a-Cuparenone | 1681 | C15H20O | 216 |
| 778 | Cyperol | 1681 | C15H24O | 220 |
| 779 | Gymnomitr-3-en-15-ol | 1688 | C15H24O | 220 |
| 780 | Pinguisanine | 1706 | C15H20O2 | 232 |
| 781 | Acora-3,7(11)-dien-8-one | 1709 | C15H22O | 218 |
| 782 | Vetiselinol | 1709 | C15H24O | 220 |
| 783 | 10,11-Dihydro-a-cuparenone | 1712 | C15H22O | 218 |
| 784 | Oxidoselina-1,3,7(11)-trien-8-one | 1725 | C15H20O2 | 232 |
| 785 | a-Sinensal | 1726 | C15H22O | 218 |
| 786 | Plagiochilide | 1729 | C15H20O2 | 232 |
| 787 | (E,E)-Methyl 10,11-epoxyfarnesoate | 1875 | C16H26O3 | 266 |
| 788 | Eudesma-3,11-dien-2-one | 1776 | C15H22O | 218 |
| 789 | Zizaenic acid | 1791 | C15H22O2 | 234 |
| 790 | Acutifolene B | 1806 | C15H20O3 | 248 |
| 791 | a-Vetivone | 1821 | C15H22O | 218 |
| 792 | (E,E)-Farnesylacetate | 1822 | C17H28O2 | 264 |
| 793 | Acutifolene A | 1833 | C16H22O3 | 262 |
| 794 | Furanoeremophilone | 1855 | C15H20O2 | 232 |
| 795 | 2-Acetoxyfuranoelemene | 1876 | C17H22O3 | 274 |
| 796 | Guaia-3,10(14)-dien-6,12-olide | 1938 | C15H20O2 | 232 |
| 797 | Guaia-3,7(11),10(14)-trien-6,12- | 1950 | C15H18O2 | 230 |
| | olide | | | |
| 798 | 1b-Acetoxyfurano-4(15)-eudesmene | 1964 | C17H22O3 | 274 |
| 799 | 1b-Acetoxyfurano-3-eudesmene | 1978 | C17H22O3 | 274 |
| 800 | Maalioxide | 1508 | C15H26O | 222 |
| 801 | Kessane | 1533 | C15H26O | 222 |
| 802 | Humulene epoxide 3 | 1626 | C15H24O | 220 |
| 803 | 8-Hydroxybicyclogermacrene | 1661 | C15H24O | 220 |
| 804 | Lactarovioline | 2068 | C15H14O | 210 |
| 805 | 5-epi-Pinguisenol | 1764 | C15H26O | 222 |
| 806 | b-Santalol acetate | 1800 | C17H26O2 | 262 |
| 807 | Bisacumol (Isomer 1) | 1596 | C15H22O | 218 |
| 808 | Bisacumol (Isomer 2) | 1619 | C15H22O | 218 |
| 809 | Bisabola-1,3(15),10-trien-9-ol | 1666 | C15H24O | 220 |
| | (Isomer 1) | | | |
| 810 | Bisabola-1,3(15),10-trien-9-ol | 1678 | C15H24O | 220 |
| | (Isomer 2) | | | |
| 811 | trans-Sesquisabinen hydrate | 1564 | C15H26O | 222 |
| 812 | 1bH-Presilphiperfolane-9a-ol | 1510 | C15H26O | 222 |
| 813 | 1aH-Presilphiperfolan-9b-ol | 1499 | C15H26O | 222 |
| 814 | Presilphiperfolane-9a-ol | 1519 | C15H26O | 222 |
| 815 | ar-Curcumen-15-al | 1681 | C15H20O | 216 |
| 816 | Sesquicineol | 1507 | C15H26O | 222 |
| 817 | Italicen-13-al | 1671 | C15H22O | 218 |
| 818 | a-Copaen-8-ol | 1551 | C15H24O | 220 |
| 819 | Khusimol | 1720 | C15H24O | 220 |
| 820 | Zizanol | 1656 | C15H24O | 220 |
| 821 | Oxidocadalene | 1644 | C15H18O | 214 |
| 822 | Eremoligenol | 1614 | C15H26O | 222 |
| 823 | Isohumbertiol D (Isomer 2) | 1519 | C15H24O | 220 |
| 824 | Isohumbertiol D (Isomer 1) | 1490 | C15H24O | 220 |
| 825 | Brachylaenalone B | 1824 | C15H20O2 | 232 |
| 826 | Khusien-12-al | 1580 | C15H22O | 218 |
| 827 | Eudesma-4(15),7(11)-dien-8-one | 1713 | C15H22O | 218 |
| 828 | Elemenone | 1589 | C15H22O | 218 |
| 829 | b-Cedrene epoxide | 1610 | C15H24O | 220 |
| 830 | b-Panasinsen-5a-ol | 1621 | C15H24O | 220 |
| 831 | Eudesm-7(11)-en-4a-ol | 1676 | C15H26O | 222 |
| 832 | 5,8-Cyclocaryophyllan-4-ol | 1514 | C15H26O | 222 |
| 833 | Khusol | 1769 | C15H24O | 220 |
| 834 | cis-10-Hydroxycalamenene | 1643 | C15H22O | 218 |
| 835 | trans-10-Hydroxycalamenene | 1635 | C15H22O | 218 |
| 836 | Bryopterine A | 1735 | C16H20O3 | 260 |
| 837 | Isoitalicene epoxide | 1501 | C15H24O | 220 |
| 838 | Italicene epoxide | 1535 | C15H24O | 220 |
| 839 | a-Agarofuran | 1537 | C15H24O | 220 |
| 840 | Longipin-3-en-10-ol | 1560 | C15H24O | 220 |
| 841 | Dihydrosesquicineol | 1467 | C15H28O | 224 |
| 842 | Dehydrosesquicineol | 1466 | C15H24O | 220 |
| 843 | Longicamphenilone | 1549 | C14H22O | 206 |
| 844 | Longicamphenilol | 1578 | C14H24O | 208 |
| 845 | Isobutyl angelate | 1027 | C9H16O2 | 156 |
| 846 | Isoacorone | 1774 | C15H24O2 | 236 |
| 847 | Dehydrosesquicineyl-12-ol | 1707 | C15H24O2 | 236 |
| 848 | Dihydrobryopterine A | 1763 | C16H22O3 | 262 |
| 849 | (E)-Nuciferal | 1705 | C15H20O | 216 |
| 850 | (Z)-Nuciferal | 1695 | C15H20O | 216 |
| 851 | Pinguisanene | 1544 | C15H20O | 216 |
| 852 | (E)-Methyl 10-hydroxy-3,7,11- | 1930 | C16H26O3 | 266 |
| | trimethyldodeca-2,6,11-trienoate | | | |
| 853 | Dihydroagarofuran | 1500 | C15H26O | 222 |
| 854 | Isolongifolol | 1717 | C15H26O | 222 |
| 855 | (Z)-Nerolidol | 1522 | C15H26O | 222 |
| 856 | (E)-Nerolidol | 1553 | C15H26O | 222 |
| 857 | a-Cedrene oxide | 1571 | C15H24O | 220 |
| 858 | Caryolan-1-ol | 1567 | C15H26O | 222 |
| 859 | Thujopsan-2a-ol | 1584 | C15H26O | 222 |
| 860 | Curcerenone | 1588 | C15H18O2 | 230 |
| 861 | a-Guaiol | 1593 | C15H26O | 222 |
| 862 | Viridiflorol | 1592 | C15H26O | 222 |
| 863 | Epicurcerenone | 1593 | C15H18O2 | 230 |
| 864 | Carotol | 1594 | C15H26O | 222 |
| 865 | Cedrol | 1603 | C15H26O | 222 |
| 866 | 12-epi-Cedrol | 1620 | C15H26O | 222 |
| 867 | 1-epi-Cubenol | 1623 | C15H26O | 222 |
| 868 | ar-Turmerone | 1643 | C15H20O | 216 |
| 869 | 3(15)-Cedren-4-ol | 1647 | C15H24O | 220 |
| 870 | a-Turmerone | 1649 | C15H22O | 218 |
| 871 | Patchouli alcohol | 1661 | C15H26O | 222 |
| 872 | (Z)-a-Santalol | 1669 | C14H22O | 206 |
| 873 | a-Bisabolol | 1673 | C15H26O | 222 |
| 874 | Acorenone B | 1679 | C15H24O | 220 |
| 875 | Germacrone | 1684 | C15H22O | 218 |
| 876 | Curcuphenol | 1693 | C15H22O | 218 |
| 877 | 2-Butylfuran | 869 | C8H12O | 124 |
| 878 | Pinguisone | 1705 | C15H20O2 | 232 |
| 879 | (Z)-b-Santalol | 1702 | C15H24O | 220 |
| 880 | Xanthorhizol | 1732 | C15H22O | 218 |
| 881 | cis-2-Hydroxycalamenene | 1762 | C15H22O | 218 |
| 882 | Furanogermenone | 1770 | C15H20O2 | 232 |
| 883 | Alantolactone | 1873 | C15H20O2 | 232 |
| 884 | Dihydroisoalantolactone | 1875 | C15H22O2 | 234 |
| 885 | Frullanolide | 1900 | C15H20O2 | 232 |
| 886 | Isoalantolactone | 1912 | C15H20O2 | 232 |
| 887 | ent-Diplophyllolide | 1937 | C15H20O2 | 232 |
| 888 | Guaia-6,9-dien-4b-ol | 1565 | C15H24O | 220 |
| 889 | Guaia-6,10(14)-diene-4b-ol | 1610 | C15H24O | 220 |
| 890 | Cedrenone | 1722 | C15H22O | 218 |
| 891 | 8bH-Cedran-9-one | 1608 | C15H24O | 220 |
| 892 | Deodarone | 1676 | C15H24O2 | 236 |
| 893 | Pogostol | 1647 | C15H26O | 222 |
| 894 | Dihydro-ar-turmerone | 1570 | C15H22O | 218 |
| 895 | Norpatchoulenol | 1551 | C14H22O | 206 |
| 896 | Caryophyllan-2,6-a-oxide | 1412 | C15H26O | 222 |
| 897 | Caryophyllen-2,6-b-oxide | 1422 | C15H26O | 222 |
| 898 | b-Atlantol (Isomer 1) | 1436 | C15H24O | 220 |
| 899 | b-Atlantol (Isomer 2) | 1443 | C15H24O | 220 |
| 900 | 1,11-Oxidocalamenene | 1474 | C15H20O | 216 |
| 901 | Furopelargone A | 1517 | C15H22O2 | 234 |
| 902 | Isohumbertiol B | 1522 | C15H24O | 220 |
| 903 | Silphiperfolene-5-ol | 1549 | C15H24O | 220 |
| 904 | b-Funebrene epoxide | 1591 | C15H24O | 220 |
| 905 | b-Himachalene epoxide | 1594 | C15H24O | 220 |
| 906 | Copaborneol | 1595 | C15H26O | 222 |
| 907 | 10-epi-Italicen-4-one | 1615 | C15H22O | 218 |
| 908 | ar-Bisabolol | 1619 | C15H22O | 218 |
| 909 | allo-Aromadendrene epoxide | 1623 | C15H240 | 220 |
| 910 | Amorph-4-en-10a-ol | 1634 | C15H26O | 222 |
| 911 | alio-Himachalol | 1648 | C15H26O | 222 |
| 912 | Farnesal (Isomer 1) | 1655 | C15H24O | 220 |
| 913 | b-Sesquiphellandrone | 1677 | C15H22O | 218 |
| 914 | Aromadendran-14-ol | 1679 | C15H26O | 222 |
| 915 | Farnesal (Isomer 2) | 1683 | C15H24O | 220 |
| 916 | Farnesal (Isomer 3) | 1707 | C15H24O | 220 |
| 917 | Longifolol | 1707 | C15H26O | 222 |
| 918 | Sesquichamaenol | 1744 | C15H22O2 | 234 |
| 919 | (E,E)-Methyl farnesoate | 1765 | C16H26O2 | 250 |
| 920 | trans-2-Hydroxycalamenene | 1753 | C15H22O | 218 |
| 921 | a-Santalol acetate | 1756 | C17H26O2 | 262 |
| 922 | 8-Acetoxyelemol | 1759 | C17H26O2 | 262 |
| 923 | Nootkatone | 1782 | C15H22O | 218 |
| 924 | Striatol | 1550 | C15H26O | 222 |
| 925 | Eremophila-1(10),11-dien-9b-ol | 1552 | C15H24O | 220 |
| 926 | Longipinanol | 1559 | C15H26O | 222 |
| 927 | Brachyl oxide | 1599 | C15H24O | 220 |
| 928 | Humulene epoxide 2 | 1602 | C15H24O | 220 |
| 929 | Copaen-15-ol | 1661 | C15H24O | 220 |
| 930 | Isonaviculol | 1743 | C15H26O | 222 |
| 931 | Cyperenal | 1741 | C15H22O | 218 |
| 932 | g-Curcumen-15-al | 1744 | C15H22O | 218 |
| 933 | Brachylaenalone A | 1802 | C15H20O2 | 232 |
| 934 | Muurola-4,10(14)-dien-8a-ol | 1594 | C15H24O | 220 |
| 935 | Cadina-1(10),4-dien-8a-ol | 1637 | C15H24O | 220 |
| 936 | Hexyl acetate | 1006 | C8H16O2 | 144 |
| 937 | Muurola-4,10(14)-dien-8b-ol | 1675 | C15H24O | 220 |
| 938 | (Z)-Nuciferol | 1695 | C15H22O | 218 |
| 939 | (Z)-g-Curcumen-12-ol | 1701 | C15H24O | 220 |
| 940 | (E)-Nuciferol | 1715 | C15H22O | 218 |
| 941 | (Z)-g-Curcumyl acetate | 1767 | C17H26O2 | 262 |
| 942 | (Z)-Nuciferyl acetate | 1793 | C17H24O2 | 260 |
| 943 | (Z)-Nuciferyl isobutyrate | 1916 | C19H28O2 | 288 |
| 944 | (Z)-g-Curcumenyl isobutyrate | 1920 | C19H30O2 | 290 |
| 945 | (Z)-Nuciferyl 2-methylbutyrate | 2003 | C20H30O2 | 302 |
| 946 | (Z)-g-Curcumyl 2-methylbutyrate | 2011 | C20H32O2 | 304 |
| 947 | Drim-8-en-7-one | 1778 | C15H24O | 220 |
| 948 | 1-Oxo-a-longipinene | 1639 | C15H22O | 218 |
| 949 | g-Bicyclohomofarnesal | 1784 | C16H26O | 234 |
| 950 | Geosmin | 1392 | C12H22O | 182 |
| 951 | Muurol-4-en-6a-ol | 1609 | C15H26O | 222 |
| 952 | Veticadine oxide | 1482 | C15H24O | 220 |
| 953 | Cubenol | 1630 | C15H26O | 222 |
| 954 | 4-epi-Cubebol | 1490 | C15H26O | 222 |
| 955 | Muurol-4-en-3,8-dione | 1753 | C15H22O2 | 234 |
| 956 | 3-Acetoxyamorpha-4,7(11)-dien-8- | 1950 | C17H24O3 | 276 |
| | one | | | |
| 957 | (E)-4,8-Dimethylnona-1,3,7-triene | 1103 | C11H18 | 150 |
| 958 | Geijerene | 1139 | C12H18 | 162 |
| 959 | Albene | 1154 | C12H18 | 162 |
| 960 | Trinoranastreptene | 1197 | C12H16 | 160 |
| 961 | 1,4a-Dimethyl-1,2,3,4,4a,5,6,7- | 1233 | C12H20 | 164 |
| | octahydro-naphthalene | | | |
| 962 | Pregeijerene | 1288 | C12H18 | 162 |
| 963 | (Z)-2,6,10-Trimethylundeca-2,6- | 1305 | C14H26 | 194 |
| | diene | | | |
| 964 | Isocyclobazzanene | 1319 | C15H24 | 204 |
| 965 | 8,9-Didehydrocycloisolongifolene | 1320 | C15H22 | 202 |
| 966 | (E)-2,6,10-Trimethylundeca-2,6- | 1321 | C14H26 | 194 |
| | diene | | | |
| 967 | Cyprotene | 1322 | C14H24 | 192 |
| 968 | Presilphiperfol-1-ene | 1325 | C15H24 | 204 |
| 969 | 7aH-Silphiperfol-5-ene | 1329 | C15H24 | 204 |
| 970 | Brasila-5,10-diene | 1335 | C15H24 | 204 |
| 971 | Bicycloax-4(15)-ene | 1336 | C15H24 | 204 |
| 972 | Bicycloelemene | 1338 | C15H24 | 204 |
| 973 | d-Elemene | 1340 | C15H24 | 204 |
| 974 | 3,10-Dihydro-1,4-dimethylazulene | 1342 | C12H14 | 158 |
| 975 | Presilphiperfol-7-ene | 1342 | C15H24 | 204 |
| 976 | Pentalenene | 1343 | C15H24 | 204 |
| 977 | African-5-ene | 1350 | C15H24 | 204 |
| 978 | African-2(6)-ene | 1350 | C15H24 | 204 |
| 979 | Maali-1,3-diene | 1347 | C15H22 | 202 |
| 980 | Silphin-1-ene | 1350 | C15H24 | 204 |
| 981 | 7bH-Silphiperfol-5-ene | 1352 | C15H24 | 204 |
| 982 | a-Cubebene | 1355 | C15H24 | 204 |
| 983 | Tamariscene | 1355 | C15H24 | 204 |
| 984 | Africa-1,5-diene | 1355 | C15H22 | 202 |
| 985 | African-1-ene | 1356 | C15H24 | 204 |
| 986 | Bicycloax-3-ene | 1357 | C15H24 | 204 |
| 987 | Silphiperfola-5,7(14)-diene | 1360 | C15H22 | 202 |
| 988 | a-Longipinene | 1360 | C15H24 | 204 |
| 989 | Clovene | 1365 | C15H24 | 204 |
| 990 | Cyperadiene | 1365 | C15H22 | 202 |
| 991 | Cyclomyltaylane | 1366 | C15H24 | 204 |
| 992 | 1-epi-a-Pinguisene | 1367 | C15H24 | 204 |
| 993 | Brasila-5(10),6-diene | 1370 | C15H24 | 204 |
| 994 | Anastreptene | 1373 | C15H22 | 202 |
| 995 | Capnell-9(12)-ene | 1372 | C15H24 | 204 |
| 996 | a-Ylangene | 1376 | C15H24 | 204 |
| 997 | Isopatchoula-3,5-diene | 1377 | C15H22 | 202 |
| 998 | Cyclosativene | 1378 | C15H24 | 204 |
| 999 | Hirsutene | 1378 | C15H24 | 204 |
| 1000 | a-Copaene | 1379 | C15H24 | 204 |
| 1001 | a-Bourbonene | 1378 | C15H24 | 204 |
| 1002 | Daucene | 1380 | C15H24 | 204 |
| 1003 | Silphiperfol-6-ene | 1378 | C15H24 | 204 |
| 1004 | Bourbon-7-ene | 1381 | C15H24 | 204 |
| 1005 | a-Elemene | 1381 | C15H24 | 204 |
| 1006 | Isodauca-4,7(14)-diene | 1381 | C15H24 | 204 |
| 1007 | Isoledene | 1382 | C15H24 | 204 |
| 1008 | Protoillud-6-ene | 1382 | C15H24 | 204 |
| 1009 | Longicyclene | 1382 | C15H24 | 204 |
| 1010 | Modhephene | 1383 | C15H24 | 204 |
| 1011 | Pacifigorgia-1(9),10-diene | 1384 | C15H24 | 204 |
| 1012 | 3-epi-African-5-ene | 1384 | C15H24 | 204 |
| 1013 | 10-epi-Italicene | 1384 | C15H24 | 204 |
| 1014 | Asterisca-3(15),6-diene | 1385 | C15H24 | 204 |
| 1015 | a-Funebrene | 1385 | C15H24 | 204 |
| 1016 | b-Panasinsene | 1385 | C15H24 | 204 |
| 1017 | Bicycloopposit-4-ene | 1386 | C15H24 | 204 |
| 1018 | b-Bourbonene | 1386 | C15H24 | 204 |
| 1019 | Isodauca-4,6-diene | 1385 | C15H24 | 204 |
| 1020 | African-2-ene | 1387 | C15H24 | 204 |
| 1021 | 7-epi-Sesquithujene | 1387 | C15H24 | 204 |
| 1022 | b-Patchoulene | 1388 | C15H24 | 204 |
| 1023 | a-Duprezianene | 1388 | C15H24 | 204 |
| 1024 | b-Elemene | 1389 | C15H24 | 204 |
| 1025 | a-Isocomene | 1389 | C15H24 | 204 |
| 1026 | 1,5-di-epi-a-Bourbonene | 1389 | C15H24 | 204 |
| 1027 | b-Cubebene | 1390 | C15H24 | 204 |
| 1028 | 1,5-di-epi-b-Bourbonene | 1390 | C15H24 | 204 |
| 1029 | African-3-ene | 1391 | C15H24 | 204 |
| 1030 | Bicyclo-4(15)-oppositene | 1391 | C15H24 | 204 |
| 1031 | Isolongifolene | 1393 | C15H24 | 204 |
| 1032 | Isodauca-6,9-diene | 1393 | C15H24 | 204 |
| 1033 | Sativene | 1394 | C15H24 | 204 |
| 1034 | Pacifigorgia-1,10-diene | 1400 | C15H24 | 204 |
| 1035 | Petasitene | 1398 | C15H24 | 204 |
| 1036 | Sesquithujene | 1399 | C15H24 | 204 |
| 1037 | African-3(15)-ene | 1400 | C15H24 | 204 |
| 1038 | Cyperene | 1402 | C15H24 | 204 |
| 1039 | b-Longipinene | 1403 | C15H24 | 204 |
| 1040 | 7-epi-a-Cedrene | 1404 | C15H24 | 204 |
| 1041 | Helifolene | 1406 | C15H24 | 204 |
| 1042 | 7-epi-Helifolene | 1406 | C15H24 | 204 |
| 1043 | Italicene | 1408 | C15H24 | 204 |
| 1044 | Isocaryophyllene | 1409 | C15H24 | 204 |
| 1045 | b-Isocomene | 1411 | C15H24 | 204 |
| 1046 | Longifolene | 1411 | C15H24 | 204 |
| 1047 | Ylanga-2,4(15)-diene | 1411 | C15H22 | 202 |
| 1048 | cis-a-Bergamotene | 1411 | C15H24 | 204 |
| 1049 | allo-Isolongifolene | 1412 | C15H24 | 204 |
| 1050 | Cycloseychellene | 1413 | C15H24 | 204 |
| 1051 | a-Gurjunene | 1413 | C15H24 | 204 |
| 1052 | a-Barbatene | 1414 | C15H24 | 204 |
| 1053 | b-Funebrene | 1418 | C15H24 | 204 |
| 1054 | b-Maaliene | 1414 | C15H24 | 204 |
| 1055 | Pacifigorgia-1(6),10-diene | 1414 | C15H24 | 204 |
| 1056 | Cascarilladiene | 1416 | C15H24 | 204 |
| 1057 | Isosativene | 1416 | C15H24 | 204 |
| 1058 | Tritomarene | 1416 | C15H24 | 204 |
| 1059 | a-Microbiotene | 1414 | C15H24 | 204 |
| 1060 | Aristolene | 1423 | C15H24 | 204 |
| 1061 | a-Cedrene | 1418 | C15H24 | 204 |
| 1062 | Pacifigorgia-2,10-diene | 1422 | C15H24 | 204 |
| 1063 | b-Ylangene | 1420 | C15H24 | 204 |
| 1064 | (Z)-b-Farnesene | 1420 | C15H24 | 204 |
| 1065 | Acora-3,5-diene | 1421 | C15H24 | 204 |
| 1066 | (E)-b-Caryophyllene | 1421 | C15H24 | 204 |
| 1067 | a-Santalene | 1422 | C15H24 | 204 |
| 1068 | Spirovetiva-1(10),6-diene | 1422 | C15H24 | 204 |
| 1069 | b-Duprezianene | 1423 | C15H24 | 204 |
| 1070 | Opposita-4(15),7-diene | 1423 | C15H24 | 204 |
| 1071 | b-Cedrene | 1424 | C15H24 | 204 |
| 1072 | Opposita-4(15),11-diene | 1424 | C15H24 | 204 |
| 1073 | Selina-3,6-diene | 1424 | C15H24 | 204 |
| 1074 | Bourbon-11-ene | 1424 | C15H24 | 204 |
| 1075 | Dauca-3,8-diene | 1428 | C15H24 | 204 |
| 1076 | Elema-1,3,7(11),8-tetraene | 1428 | C15H22 | 202 |
| 1077 | g-Elemene | 1429 | C15H24 | 204 |
| 1078 | Isobarbatene | 1428 | C15H24 | 204 |
| 1079 | g-Maaliene | 1428 | C15H24 | 204 |
| 1080 | Aristola-1(10),8-diene | 1429 | C15H22 | 202 |
| 1081 | Chenopodene | 1430 | C15H24 | 204 |
| 1082 | b-Copaene | 1430 | C15H24 | 204 |
| 1083 | Thujopsene | 1434 | C15H24 | 204 |
| 1084 | Selina-4(15),5-diene | 1433 | C15H24 | 204 |
| 1085 | Pacifigorgia-2(10),11-diene | 1435 | C15H24 | 204 |
| 1086 | trans-a-Bergamotene | 1434 | C15H24 | 204 |
| 1087 | a-Pinguisene | 1436 | C15H24 | 204 |
| 1088 | b-Sesquifenchene | 1437 | C15H24 | 204 |
| 1089 | Sesquisabinene A | 1435 | C15H24 | 204 |
| 1090 | Calarene | 1437 | C15H24 | 204 |
| 1091 | Cubeb-11-ene | 1445 | C15H24 | 204 |
| 1092 | b-Gorgonene | 1440 | C15H24 | 204 |
| 1093 | a-Maalinene | 1440 | C15H24 | 204 |
| 1094 | Cyclofarnesa-5(14),8,10-triene | 1441 | C15H24 | 204 |
| 1095 | a-Guaiene | 1440 | C15H24 | 204 |
| 1096 | Acora-3,9-diene | 1442 | C15H24 | 204 |
| 1097 | Aromadendrene | 1443 | C15H24 | 204 |
| 1098 | Brasila-1(6),5(10)-diene | 1442 | C15H24 | 204 |
| 1099 | Isobazzanene | 1442 | C15H24 | 204 |
| 1100 | Guaia-6,9-diene | 1443 | C15H24 | 204 |
| 1101 | Nardosina-7,9,11-triene | 1444 | C15H22 | 202 |
| 1102 | 4aH,10aH-Guaia-1(5),6-diene | 1445 | C15H24 | 204 |
| 1103 | Isogermacrene D | 1445 | C15H24 | 204 |
| 1104 | Selina-5,11-diene | 1444 | C15H24 | 204 |
| 1105 | Eremophila-1(10),6-diene | 1445 | C15H24 | 204 |
| 1106 | b-Barbatene | 1445 | C15H24 | 204 |
| 1107 | Cadina-4,11-diene | 1458 | C15H24 | 204 |
| 1108 | Erythrodiene | 1446 | C15H24 | 204 |
| 1109 | epi-b-Santalene | 1446 | C15H24 | 204 |
| 1110 | Sesquisabinene B | 1446 | C15H24 | 204 |
| 1111 | Seychellene | 1447 | C15H24 | 204 |
| 1112 | Cadina-3,5-diene | 1448 | C15H24 | 204 |
| 1113 | (E)-b-Farnesene | 1446 | C15H24 | 204 |
| 1114 | 4bH,10aH-Guaia-1(5),6-diene | 1448 | C15H24 | 204 |
| 1115 | Selina-4(15),6-diene | 1450 | C15H24 | 204 |
| 1116 | a-Himachalene | 1450 | C15H24 | 204 |
| 1117 | Prezizaene | 1452 | C15H24 | 204 |
| 1118 | Bourbon-7(11)-ene | 1454 | C15H24 | 204 |
| 1119 | a-Humulene | 1455 | C15H24 | 204 |
| 1120 | e-Muurolene | 1455 | C15H24 | 204 |
| 1121 | a-Panasinsene | 1455 | C15H24 | 204 |
| 1122 | Zizaene | 1456 | C15H24 | 204 |
| 1123 | a-Neoclovene | 1456 | C15H24 | 204 |
| 1124 | Valerena-4,7(11)-diene | 1456 | C15H24 | 204 |
| 1125 | Acora-3,10(14)-diene | 1457 | C15H24 | 204 |
| 1126 | Selina-4(15),7-diene | 1457 | C15H24 | 204 |
| 1127 | b-Spathulene | 1457 | C15H22 | 202 |
| 1128 | Muurola-4,11-diene | 1458 | C15H24 | 204 |
| 1129 | Selina-2,4-diene | 1462 | C15H24 | 204 |
| 1130 | (Z,Z)-a-Farnesene | 1460 | C15H24 | 204 |
| 1131 | b-Santalene | 1460 | C15H24 | 204 |
| 1132 | 7bH,10bH-Cadina-1(6),4-diene | 1460 | C15H24 | 204 |
| 1133 | Rotundene | 1461 | C15H24 | 204 |
| 1134 | Selina-3,7-diene | 1460 | C15H24 | 204 |
| 1135 | Striatene | 1458 | C15H24 | 204 |
| 1136 | allo-Aromadendrene | 1462 | C15H24 | 204 |
| 1137 | Aromadendr-9-ene | 1463 | C15H24 | 204 |
| 1138 | a-Patchoulene | 1467 | C15H24 | 204 |
| 1139 | a-Acoradiene | 1464 | C15H24 | 204 |
| 1140 | Carota-5,8-diene | 1465 | C15H24 | 204 |
| 1141 | b-Acoradiene | 1465 | C15H24 | 204 |
| 1142 | 4,5-di-epi-Aristolochene | 1470 | C15H24 | 204 |
| 1143 | Selina-4,7-diene | 1469 | C15H24 | 204 |
| 1144 | 2-epi-(E)-b-Caryophyllene | 1467 | C15H24 | 204 |
| 1145 | g-Muurolene | 1474 | C15H24 | 204 |
| 1146 | Amorpha-4,11-diene | 1472 | C15H24 | 204 |
| 1147 | 7aH,10bH-Cadina-1(6),4-diene | 1472 | C15H24 | 204 |
| 1148 | ar-Curcumene | 1473 | C15H22 | 202 |
| 1149 | Eudesma-1,4(15),11-triene | 1472 | C15H22 | 202 |
| 1150 | Eudesma-2,4,11-triene | 1471 | C15H22 | 202 |
| 1151 | g-Gurjunene | 1472 | C15H24 | 204 |
| 1152 | Ishwarane | 1468 | C15H24 | 204 |
| 1153 | Valenca-2,9,11-trIene | 1473 | C15H22 | 202 |
| 1154 | b-Chamigrene | 1474 | C15H24 | 204 |
| 1155 | (3E,6Z)-a-Farnesene | 1475 | C15H24 | 204 |
| 1156 | Selina-4,11-diene | 1475 | C15H24 | 204 |
| 1157 | b-Microbiotene | 1473 | C15H24 | 204 |
| 1158 | a-Amorphene | 1477 | C15H24 | 204 |
| 1159 | g-Curcumene | 1475 | C15H24 | 204 |
| 1160 | Herbertene | 1476 | C15H22 | 202 |
| 1161 | Zierene | 1476 | C15H22 | 202 |
| 1162 | a-Neocallitropsene | 1475 | C15H24 | 204 |
| 1163 | Amorpha-4,7(11)-diene | 1476 | C15H24 | 204 |
| 1164 | 5-epi-Aristolochene | 1477 | C15H24 | 204 |
| 1165 | Isobicyclogermacrene | 1477 | C15H24 | 204 |
| 1166 | b-Neoclovene | 1475 | C15H24 | 204 |
| 1167 | trans-b-Bergamotene | 1480 | C15H24 | 204 |
| 1168 | g-Himachalene | 1479 | C15H24 | 204 |
| 1169 | Laurene | 1483 | C15H20 | 200 |
| 1170 | Germacrene D | 1479 | C15H24 | 204 |
| 1171 | (3Z,6E)-a-Farnesene | 1480 | C15H24 | 204 |
| 1172 | a-Vetispirene | 1481 | C15H22 | 202 |
| 1173 | e-Cadinene | 1483 | C15H24 | 204 |
| 1174 | g-Humulene | 1483 | C15H24 | 204 |
| 1175 | Isolepidozene | 1483 | C15H24 | 204 |
| 1176 | cis-Eudesma-6,11-diene | 1484 | C15H24 | 204 |
| 1177 | Nardosina-9,11-diene | 1484 | C15H24 | 204 |
| 1178 | Nardosina-1(10),11-diene | 1484 | C15H24 | 204 |
| 1179 | Eudesma-3,5,11-triene | 1485 | C15H22 | 202 |
| 1180 | Aristolochene | 1486 | C15H24 | 204 |
| 1181 | Eremophilene | 1486 | C15H24 | 204 |
| 1182 | d-Selinene | 1490 | C15H24 | 204 |
| 1183 | b-Vetispirene | 1486 | C15H22 | 202 |
| 1184 | Bicyclosesquiphellandrene | 1487 | C15H24 | 204 |
| 1185 | b-Selinene | 1486 | C15H24 | 204 |
| 1186 | g-Amorphene | 1492 | C15H24 | 204 |
| 1187 | allo-Aromadendr-9-ene | 1489 | C15H24 | 204 |
| 1188 | Eremophila-1(10),7-diene | 1488 | C15H24 | 204 |
| 1189 | Selina-3,5-diene | 1486 | C15H24 | 204 |
| 1190 | Zingiberene | 1489 | C15H24 | 204 |
| 1191 | b-Alaskene | 1495 | C15H24 | 204 |
| 1192 | Ledene | 1491 | C15H24 | 204 |
| 1193 | Drim-8(12)-ene | 1497 | C15H26 | 206 |
| 1194 | Valencene | 1494 | C15H24 | 204 |
| 1195 | epi-Zonarene | 1494 | C15H24 | 204 |
| 1196 | (Z)-a-Bisabolene | 1494 | C15H24 | 204 |
| 1197 | a-Selinene | 1494 | C15H24 | 204 |
| 1198 | Bicyclogermacrene | 1494 | C15H24 | 204 |
| 1199 | Caparratriene | 1493 | C15H26 | 206 |
| 1200 | Eudesma-2,4(15),11-triene | 1495 | C15H22 | 202 |
| 1201 | Hinesene | 1495 | C15H24 | 204 |
| 1202 | a-Muurolene | 1496 | C15H24 | 204 |
| 1203 | Aciphyllene | 1495 | C15H24 | 204 |
| 1204 | a-Cuprenene | 1497 | C15H24 | 204 |
| 1205 | Cuparene | 1498 | C15H22 | 202 |
| 1206 | g-Patchoulene | 1497 | C15H24 | 204 |
| 1207 | e-Amorphene | 1498 | C15H24 | 204 |
| 1208 | g-Guaiene | 1499 | C15H24 | 204 |
| 1209 | b-Pinguisene | 1500 | C15H24 | 204 |
| 1210 | d-Amorphene | 1499 | C15H24 | 204 |
| 1211 | (E,E)-a-Farnesene | 1498 | C15H24 | 204 |
| 1212 | 1aH,10aH-Guaia-4,6-diene | 1500 | C15H24 | 204 |
| 1213 | b-Himachalene | 1500 | C15H24 | 204 |
| 1214 | D7(14)-ar-Himachalene | 1501 | C15H20 | 200 |
| 1215 | b-Bisabolene | 1503 | C15H24 | 204 |
| 1216 | a-Chamigrene | 1503 | C15H24 | 204 |
| 1217 | Eremophila-1(10),8,11-triene | 1504 | C15H22 | 202 |
| 1218 | Germacrene A | 1503 | C15H24 | 204 |
| 1219 | Isorotundene | 1503 | C15H24 | 204 |
| 1220 | a-Bulnesene | 1503 | C15H24 | 204 |
| 1221 | b-Curcumene | 1503 | C15H24 | 204 |
| 1222 | Drimenene | 1503 | C15H24 | 204 |
| 1223 | Pseudowiddrene | 1503 | C15H24 | 204 |
| 1224 | a-Alaskene | 1512 | C15H24 | 204 |
| 1225 | (Z)-g-Bisabolene | 1505 | C15H24 | 204 |
| 1226 | g-Cadinene | 1507 | C15H24 | 204 |
| 1227 | Nootkatene | 1512 | C15H22 | 202 |
| 1228 | Cyclobazzanene | 1514 | C15H24 | 204 |
| 1229 | cis-Calamenene | 1517 | C15H22 | 202 |
| 1230 | b-Sesquiphellandrene | 1516 | C15H24 | 204 |
| 1231 | D7,8-ar-Himachalene | 1518 | C15H20 | 200 |
| 1232 | 7-epi-a-Selinene | 1519 | C15H24 | 204 |
| 1233 | b-Bazzanene | 1519 | C15H24 | 204 |
| 1234 | d-Cadinene | 1520 | C15H24 | 204 |
| 1235 | (E)-g-Bisabolene | 1521 | C15H24 | 204 |
| 1236 | trans-Calamenene | 1517 | C151122 | 202 |
| 1237 | Zonarene | 1521 | C15H24 | 204 |
| 1238 | b-Cadinene | 1526 | C15H24 | 204 |
| 1239 | g-Cuprenene | 1523 | C15H24 | 204 |
| 1240 | Spirovetiva-1(10),7(11)-diene | 1523 | C15H24 | 204 |
| 1241 | g-Vetivenene | 1525 | C15H22 | 202 |
| 1242 | Cadina-1,4-diene | 1523 | C15H24 | 204 |
| 1243 | w-Cadinene | 1526 | C15H24 | 204 |
| 1244 | a-Calacorene | 1527 | C15H20 | 200 |
| 1245 | Eremophila-1(10),7(11)-diene | 1527 | C15H24 | 204 |
| 1246 | ar-Himachalene | 1528 | C15H22 | 202 |
| 1247 | (E)-a-Bisabolene | 1530 | C15H24 | 204 |
| 1248 | 5-epi-Laurene | 1531 | C15H20 | 200 |
| 1249 | 1,4-Dimethylazulene | 1532 | C12H12 | 156 |
| 1250 | Selina-4(15),7(11)-diene | 1534 | C15H24 | 204 |
| 1251 | a-Cadinene | 1534 | C15H24 | 204 |
| 1252 | w-Amorphene | 1540 | C15H24 | 204 |
| 1253 | d-Cuprenene | 1546 | C15H24 | 204 |
| 1254 | (1(10)E,4Z)-Germacrene B | 1543 | C15H24 | 204 |
| 1255 | Selina-3,7(11)-diene | 1542 | C15H24 | 204 |
| 1256 | Germacrene B | 1552 | C15H24 | 204 |
| 1257 | b-Vetivenene | 1552 | C15H22 | 202 |
| 1258 | g-Calacorene | 1554 | C15H20 | 200 |
| 1259 | (3E,7E)-4,8,12-Trimethyltrideca- | 1565 | C16H26 | 218 |
| | 1,3,7,11-tetraene | | | |
| 1260 | Cadalene | 1659 | C15H18 | 198 |
| 1261 | Daucalene | 1671 | C15H18 | 198 |
| 1262 | Chamazulene | 1719 | C14H16 | 184 |
| 1263 | Guaiazulene | 1761 | C15H18 | 198 |
| 1264 | 6-epi-b-Cubebene | 1449 | C15H24 | 204 |
| 1265 | e-Cuprenene | 1524 | C15H24 | 204 |
| 1266 | Gymnomitra-3(15),4-diene | 1413 | C15H22 | 202 |
| 1267 | Tenuifolene | 1570 | C15H22 | 202 |
| 1268 | ar-Tenuifolene | 1528 | C15H20 | 200 |
| 1269 | trans-Eudesma-3,5-diene | 1490 | C15H24 | 204 |
| 1270 | Pethybrene | 1440 | C15H24 | 204 |
| 1271 | Premnaspirodiene | 1516 | C15H24 | 204 |
| 1272 | Spirolepechinene | 1450 | C15H24 | 204 |
| 1273 | trans-Dauca-4(11),7-diene | 1554 | C15H24 | 204 |
| 1274 | trans-Dauca-4(11),8-diene | 1529 | C15H24 | 204 |
| 1275 | Cadina-1(10),3,7(11)-triene | 1575 | C15H22 | 202 |
| 1276 | 7,8-Dehydro-a-acoradiene | 1450 | C15H22 | 202 |
| 1277 | cis-Muurola-4(15),5-diene | 1462 | C15H24 | 204 |
| 1278 | Patchoula-2,4(15)-diene | 1434 | C15H22 | 202 |
| 1279 | Norrotundene | 1421 | C14H22 | 190 |
| 1280 | cis-b-Guaiene | 1488 | C15H24 | 204 |
| 1281 | Bisaboia-1,3,5,11-tetraene | 1461 | C15H24 | 204 |
| 1282 | 4-epi-b-Patchoulene | 1376 | C15H24 | 204 |
| 1283 | d-Patchoulene | 1466 | C15H24 | 204 |
| 1284 | e-Patchoulene | 1473 | C15H24 | 204 |
| 1285 | 10-epi-Muurola-4,11-diene | 1458 | C15H24 | 204 |
| 1286 | Dauca-8,11-diene | 1431 | C15H24 | 204 |
| 1287 | Neotrifaradiene | 1365 | C15H24 | 204 |
| 1288 | Sandvicene | 1399 | C15H24 | 204 |
| 1289 | Trifara-9,14-diene | 1403 | C15H24 | 204 |
| 1290 | cis-Muurola-3,5-diene | 1447 | C15H24 | 204 |
| 1291 | Pacifigorgia-6,10-diene | 1429 | C15H24 | 204 |
| 1292 | b-Bulnesene | 1558 | C15H24 | 204 |
| 1293 | Isocalamenene | 1527 | C15H22 | 202 |
| 1294 | Myltayl-4(12)-ene | 1452 | C15H24 | 204 |
| 1295 | 3,7-di-epi-Trifara-9,14-diene | 1399 | C15H24 | 204 |
| 1296 | 6-epi-a-Cubebene | 1418 | C15H24 | 204 |
| 1297 | 2-Sterpurene | 1351 | C15H24 | 204 |
| 1298 | a-Corocalen | 1602 | C15H20 | 200 |
| 1299 | Lactarazulene | 1796 | C15H16 | 196 |
| 1300 | Prenyllimonene (Isomer 1) | 1436 | C15H24 | 204 |
| 1301 | Prenyllimonene (Isomer 2) | 1450 | C15H24 | 204 |
| 1302 | Cadina-1(10),7(11)-diene | 1538 | C15H24 | 204 |
| 1303 | Elema-1,3,7-triene | 1346 | C15H24 | 204 |
| 1304 | 7-epi-Cadina-1(10),11-diene | 1525 | C15H24 | 204 |
| 1305 | Cadina-1(10),11-diene | 1480 | C15H24 | 204 |
| 1306 | Vetivazulene | 1790 | C15H18 | 198 |
| 1307 | Mintsulphide | 1734 | C15H24S | 236 |
| 1308 | Brasila-1,10-diene | 1307 | C15H24 | 204 |
| 1309 | Drim-8-ene | 1442 | C15H26 | 206 |
| 1310 | Selina-4(15),7,11-triene | 1469 | C15H22 | 202 |
| 1311 | 5,6-Dehydroalaskene | 1371 | C15H22 | 202 |
| 1312 | (all-Z)-6,9,12,15-Heneicosatetraene | 2048 | C21H36 | 288 |
| 1313 | Isoperillene | 1073 | C10H14O | 150 |
| 1314 | (E)-Cinnamyl isovalerate | 1641 | C14H18O2 | 218 |
| 1315 | (E)-Cinnamyl isobutyrate | 1543 | C13H16O2 | 204 |
| 1316 | (E)-Cinnamyl propionate | 1500 | C12H14O2 | 190 |
| 1317 | (Z)-Isobutyl cinnamate | 1593 | C13H16O2 | 204 |
| 1318 | Phenylethyl tiglate | 1547 | C13H16O2 | 204 |
| 1319 | 7-epi-Eremophila-1(10),8,11-triene | 1508 | C15H22 | 202 |
| 1320 | 5-Hydroxymarsupellyl acetate | 1814 | C17H26O3 | 278 |
| 1321 | Marsupellyl acetate | 1681 | C17H26O2 | 262 |
| 1322 | 4-epi-Marsupellyl acetate | 1733 | C17H26O2 | 262 |
| 1323 | (E)-Methyl p-methoxycinnamate | 1625 | C11H12O3 | 192 |
| 1324 | (Z)-Methyl p-methoxycinnamate | 1543 | C11H12O3 | 192 |
| 1325 | 4-epi-Marsupellol | 1614 | C15H24O | 220 |
| 1326 | (Z)-Cinnamyl propionate | 1552 | C12H14O2 | 190 |
| 1327 | (E)-Isobutyl cinnamate | 1633 | C13H16O2 | 204 |
| 1328 | Methyl 4-methoxymandelate | 1511 | C10H12O4 | 196 |
| 1329 | (E)-Isoamyl cinnamate | 1697 | C14H18O2 | 218 |
| 1330 | Pentadecanoic acid | 1823 | C15H30O2 | 242 |
| 1331 | Methyl o-methoxybenzoate | 1300 | C9H10O3 | 166 |
| 1332 | Patchenol | 1305 | C11H18O | 166 |
| 1333 | Syringa aldehyde | 1599 | C9H10O4 | 182 |
| 1334 | Methyl 3-methylorsellinate | 1674 | C10H12O4 | 196 |
| 1335 | Dihydroactinidiolide | 1487 | C11H16O2 | 180 |
| 1336 | b-Ionone epoxide | 1460 | C13H20O2 | 208 |
| 1337 | Oxoisophorone | 1111 | C9H12O2 | 152 |
| 1338 | Sabina ketone | 1132 | C9H14O | 138 |
| 1339 | 2,6-Di-tert-butyl-4-methylphenol | 1492 | C15H24O | 220 |
| 1340 | Cadin-1(10)-ene 5,11-oxide | 1574 | C15H24O | 220 |
| 1341 | 6,11-Epoxyisodaucane | 1463 | C15H26O | 222 |
| 1342 | 3-Acetoxy-b-ionone | 1752 | C15H22O3 | 250 |
| 1343 | Nardosina-7,9-dien-11-ol | 1596 | C15H24O | 220 |
| 1344 | Porosadienol | 1627 | C15H24O | 220 |
| 1345 | a-Ionone epoxide (Isomer 2) | 1512 | C13H20O2 | 208 |
| 1346 | Cabreuva oxide A | 1437 | C15H24O | 220 |
| 1347 | Cabreuva oxide B | 1452 | C15H24O | 220 |
| 1348 | Cabreuva oxide C | 1456 | C15H24O | 220 |
| 1349 | (E)-o-Methoxycinnamaldehyde | 1477 | C10H10O2 | 162 |
| 1350 | (Z)-o-Methoxycinnamaldehyde | 1408 | C10H10O2 | 162 |
| 1351 | Hydrocinnamyl acetate | 1336 | C11H14O2 | 178 |
| 1352 | N-Methyl methyl anthranilate | 1372 | C9H11O2N | 165 |
| 1353 | Abietal | 2261 | C20H30O | 286 |
| 1354 | trans-Totarol | 2241 | C20H30O | 286 |
| 1355 | Dehydrogeosmin | 1362 | C12H20O | 180 |
| 1356 | 1bH,5aH,7bH-Guaia-3,10(14)-dien- | 1646 | C15H24O | 220 |
| | 11-ol | | | |
| 1357 | 9a,11-Epoxy-1bH,5aH,7bH,9bH- | 1587 | C15H22O | 218 |
| | guaia-3,10(14)-diene | | | |
| 1358 | 4-(4-Hydroxyphenyl)-2-butanone | 1508 | C10H12O2 | 164 |
| 1359 | 15-Norlabdan-8-ol | 1943 | C19H36O | 280 |
| 1360 | Oxoisoambrox | 1819 | C16H26O2 | 250 |
| 1361 | Sclareolide | 2022 | C16H24O3 | 264 |
| 1362 | 1-Decanol | 1264 | C10H22O | 158 |
| 1363 | Amberone | 1810 | C17H26O | 246 |
| 1364 | Methyl arachidonate | 2217 | C21H34O2 | 318 |
| 1365 | Cyclomyltaylan-15-ol | 1641 | C15H24O | 220 |
| 1366 | Tridenson | 1633 | C15H26O | 222 |
| 1367 | Tridensenal | 1617 | C15H26O | 222 |
| 1368 | 6b-Acetoxyeudesm-4(15)-en-7b-ol | 1898 | C18H30O2 | 278 |
| 1369 | Tridensenone | 1815 | C15H20O | 216 |
| 1370 | 2,6,6-Trimethylcyclohexanone | 1023 | C9H16O | 140 |
| 1371 | 2,6,6-Trimethylcyclohex-2-enone | 1045 | C9H14O | 138 |
| 1372 | Acetoxycedren-13-ol | 1782 | C17H26O2 | 262 |
| 1373 | 4-Isopropylcyclohexanol (Isomer 1) | 1126 | C9H18O | 142 |
| 1374 | 3-Hydroxy-4-methoxybenzyl | 1421 | C8H10O3 | 154 |
| | alcohol | | | |
| 1375 | a-Ambrinol (Isomer 1) | 1382 | C13H22O | 194 |
| 1376 | a-Ambrinol (Isomer 2) | 1410 | C13H22O | 194 |
| 1377 | Thymohydroquinone | 1509 | C10H14O2 | 166 |
| 1378 | Oreodaphnenol | 1484 | C15H24O | 220 |
| 1379 | Ambrox | 1747 | C16H28O | 236 |
| 1380 | 4-Isopropylphenol | 1201 | C9H12O | 136 |
| 1381 | Scopoletine | 1888 | C10H8O4 | 192 |
| 1382 | 2,5-Dimethoxy-4-isopropyltoluene | 1400 | C12H18O2 | 194 |
| 1383 | Silphiperfol-5-en-3-one | 1533 | C15H22O | 218 |
| 1384 | Clovenol | 1575 | C15H24O | 220 |
| 1385 | trans-6-Hydroxyisocalamenene | 1782 | C15H22O | 218 |
| 1386 | 1,4-trans-6-Methoxyisocalamenene | 1722 | C16H24O | 232 |
| 1387 | Non-1-ene | 837 | C9H18 | 126 |
| 1388 | Mintoxide | 1565 | C15H24O | 220 |
| 1389 | 6-Methylheptan-2,4-dione | 975 | C8H14O2 | 142 |
| 1390 | 5-Methylheptan-2,4-dione | 966 | C8H14O2 | 142 |
| 1391 | 2,2-Dimethyl-7-isobutyl-2H,5H- | 1770 | C14H18O3 | 234 |
| | pyrano[4.3-b]pyran-5-one | | | |
| 1392 | 2,2-Dimethyl-7-secbutyl-2H,5H- | 1764 | C14H18O3 | 234 |
| | pyrano[4.3-b]pyran-5-one | | | |
| 1393 | Cyclo-b-ionone | 1329 | C13H20O | 192 |
| 1394 | Germacra-4(15),5,10(14)-trien-1a-ol | 1680 | C15H24O | 220 |
| 1395 | Eudesma-4(15),7-dien-1b-ol | 1671 | C15H24O | 220 |
| 1396 | Cadina-4,10(14)-dien-1a-ol | 1662 | C15H24O | 220 |
| 1397 | b-Calacorene | 1541 | C15H20 | 200 |
| 1398 | 1a,10a-Epoxyamorph-4-ene | 1569 | C15H24O | 220 |
| 1399 | Muurola-4,10(14)-dien-1-ol | 1626 | C15H24O | 220 |
| 1400 | Caryophylla-3(15),7(14)-dien-6-ol | 1635 | C15H24O | 220 |
| 1401 | 4(15)-Dehydroglobulol | 1597 | C15H24O | 220 |
| 1402 | trans-Bisabola-1(6),10-dien-2,3-diol | 1758 | C15H26O2 | 238 |
| 1403 | 6,10-Epoxybisabol-2-en-12-al | 1664 | C15H24O2 | 236 |
| 1404 | 6,10-Epoxybisabol-3-en-12-al | 1677 | C15H24O2 | 236 |
| 1405 | 11-epi-6,10-Epoxybisabol-3-en-12- | 1649 | C15H24O2 | 236 |
| | al | | | |
| 1406 | Acora-3,5-dien-11-ol | 1574 | C15H24O | 220 |
| 1407 | Acora-2,4(15)-dien-11-ol | 1616 | C15H24O | 220 |
| 1408 | 7-epi-Bisabol-1-one | 1718 | C15H24O | 220 |
| 1409 | (E)-trans-a-Bergamota-2,10-dien-12- | 1679 | C15H22O | 218 |
| | al | | | |
| 1410 | Helifolen-12-al (syn-syn-syn) | 1611 | C16H24O | 232 |
| 1411 | Italicene ether | 1531 | C15H24O | 220 |
| 1412 | 7-epi-b-Bisabolol | 1657 | C15H26O | 222 |
| 1413 | Bisabol-1-one | 1712 | C15H24O | 220 |
| 1414 | Humulene epoxide 1 | 1593 | C14H22O | 206 |
| 1415 | 10-epi-Italicene ether | 1511 | C15H24O | 220 |
| 1416 | 3-Hydroxybisabola-1(6),10-dien-2- | 1748 | C15H24O2 | 236 |
| | one | | | |
| 1417 | b-Bisabolol | 1659 | C15H26O | 222 |
| 1418 | 10-epi-Junenol | 1581 | C15H26O | 222 |
| 1419 | Junenol | 1617 | C15H26O | 222 |
| 1420 | 1,10-di-epi-Cubenol | 1615 | C15H26O | 222 |
| 1421 | Carquejyl acetate | 1284 | C12H16O2 | 192 |
| 1422 | (E)-Dendrolasin | 1566 | C15H22O | 218 |
| 1423 | Artemisyl acetate | 1164 | C12H20O2 | 196 |
| 1424 | Artedouglasia oxide C | 1507 | C15H22O3 | 250 |
| 1425 | Artedouglasia oxide A | 1517 | C15H22O3 | 250 |
| 1426 | 1-Undecanol | 1363 | C11H24O | 172 |
| 1427 | Laciniata furanone H | 1530 | C15H22O3 | 250 |
| 1428 | Lanciniata furanone F | 1514 | C15H22O3 | 250 |
| 1429 | Artedouglasia oxide B | 1561 | C15H22O3 | 250 |
| 1430 | Artedouglasia oxide D | 1542 | C15H22O3 | 250 |
| 1431 | Cymen-8-ol | 1169 | C10H14O | 150 |
| 1432 | 2,2,9-Trimethyl-1,6- | 1079 | C10H14O2 | 166 |
| | dioxaspiro[4.4]nona-3,8-diene | | | |
| 1433 | Menthyl formate | 1230 | C11H20O2 | 184 |
| 1434 | Folifolone | 1090 | C10H14O | 150 |
| 1435 | Santolina alcohol | 1029 | C10H18O | 154 |
| 1436 | cis-p-Mentha-1(7),8-dien-2-ol | 1217 | C10H16O | 152 |
| 1437 | trans-p-Mentha-1(7),8-dien-2-ol | 1176 | C10H16O | 152 |
| 1438 | trans-p-Menth-2-en-1-ol | 1116 | C10H18O | 154 |
| 1439 | cis-p-Mentha-2,8-dien-1-ol | 1125 | C10H16O | 152 |
| 1440 | trans-p-Mentha-2,8-dien-1-ol | 1113 | C10H16O | 152 |
| 1441 | Dehydrosabinaketone | 1100 | C9H12O | 136 |
| 1442 | 4-Hydroxy-4-methylcyclohex-2- | 1089 | C7H10O2 | 126 |
| | enone | | | |
| 1443 | 2-(1-Hydroxyethyl)-5-methyl-5- | 1054 | C9H16O2 | 156 |
| | vinyltetrahydrofuran | | | |
| 1444 | trans-Arbusculone | 1036 | C9H14O2 | 154 |
| 1445 | Lavender lactone | 1006 | C7H10O2 | 126 |
| 1446 | Pulegone epoxide | 1238 | C10H16O2 | 168 |
| 1447 | 3-Methylcyclohexanone | 928 | C7H12O | 112 |
| 1448 | trans-Linalool oxide acetate | 1274 | C12H20O3 | 212 |
| 1449 | Fragranyl acetate | 1331 | C11H18O2 | 182 |
| 1450 | 6-Methyl-6-(3-methylphenyl)-2- | 1609 | C15H22O | 218 |
| | heptanone | | | |
| 1451 | 3-exo-Acetoxybornyl acetate | 1520 | C14H22O4 | 254 |
| 1452 | 3-exo-Acetoxyborneol | 1402 | C12H20O3 | 212 |
| 1453 | 3-exo-Hydroxybornyl acetate | 1393 | C12H20O3 | 212 |
| 1454 | Lavandulyl acetate | 1275 | C12H20O2 | 196 |
| 1455 | 5-Hydroxymarsupellol | 1776 | C15H24O2 | 236 |
| 1456 | b-Isolongibornene | 1440 | C15H24 | 204 |
| 1457 | Geranyl propionate | 1486 | C13H22O2 | 210 |
| 1458 | (E)-Isosafrol | 1356 | C10H10O2 | 162 |
| 1459 | 2,3-Dihydrofarnesol | 1674 | C15H28O | 224 |
| 1460 | Methyl 4-hydroxymandelate | 1572 | C9H10O4 | 182 |
| 1461 | Methyl 3-(4-methoxyphenyl)- | 1494 | C11H14O3 | 194 |
| | propionate | | | |
| 1462 | Methyl 3,5-dimethoxyphenylacetate | 1603 | C11H14O4 | 210 |
| 1463 | 2a-Hydroxyamorpha-4,7(11)-diene | 1678 | C15H24O | 220 |
| 1464 | l-Hepten-3-one | 956 | C7H12O | 112 |
| 1465 | Ferulyl angelate | 1682 | C15H20O3 | 248 |
| 1466 | Undecanal | 1290 | C11H22O | 170 |
| 1467 | n-Hexadecanoic acid | 1951 | C16H32O2 | 256 |
| 1468 | n-Tetradecanoic acid | 1748 | C14H28O2 | 228 |
| 1469 | n-Dodecanoic acid | 1554 | C12H24O2 | 200 |
| 1470 | n-Decanal | 1180 | C10H20O | 156 |
| 1471 | Axenol (Gleenol) | 1574 | C15H26O | 222 |
| 1472 | epi-Methyl jasmonate | 1637 | C13H20O3 | 224 |
| 1473 | 5-Ethylcyclopent-1-enecarbaldehyde | 1010 | C8H12O | 124 |
| 1474 | Methyl hexanoate | 905 | C7H14O2 | 130 |
| 1475 | Methyl undecanoate | 1400 | C12H24O2 | 200 |
| 1476 | Methyl dodecanoate | 1500 | C13H26O2 | 214 |
| 1477 | Methyl tridecanoate | 1600 | C14H28O2 | 228 |
| 1478 | Methyl myristoleate | 1683 | C15H28O2 | 240 |
| 1479 | (Z)-Methyl pentadec-10-enoate | 1786 | C16H30O2 | 254 |
| 1480 | Methyl palmitoleate | 1877 | C17H32O2 | 268 |
| 1481 | (Z)-Methyl Heptadec-10-enoate | 1978 | C18H34O2 | 282 |
| 1482 | Methyl heptadecanoate | 2001 | C17H34O2 | 270 |
| 1483 | Methyl oleate | 2082 | C19H36O2 | 296 |
| 1484 | Dihydroedulan | 1290 | C13H22O | 194 |
| 1485 | 2-Methylbenzofuran | 1149 | C9H8O | 132 |
| 1486 | (all-Z)-Methyl Docosa- | 2395 | C23H34O2 | 342 |
| | 4,7,10,13,16,19-hexaenoate | | | |
| 1487 | (Z,Z)-Methyl docosa-13,16-dienoate | 2433 | C23H42O2 | 350 |
| 1488 | Methyl erucate | 2440 | C23H44O2 | 352 |
| 1489 | Methyl behenate | 2459 | C23H46O2 | 354 |
| 1490 | Methyl tricosanoate | 2558 | C24H48O2 | 368 |
| 1491 | Methyl nervonate | 2650 | C25H48O2 | 380 |
| 1492 | (Z)-Methyl eicosa-11-enoate | 2248 | C21H40O2 | 324 |
| 1493 | Methyl lignocerate | 2695 | C25H50O2 | 382 |
| 1494 | Methyl arachidate | 2306 | C21H42O2 | 326 |
| 1495 | Methyl heneicosanoate (C-21) | 2412 | C22H44O2 | 340 |
| 1496 | Methyl stearate | 2104 | C19H38O2 | 298 |
| 1497 | (all-Z)-Methyl eicosa-11,14-dienoate | 2243 | C22H40O2 | 336 |
| 1498 | Methyl elaidate | 2084 | C19H36O2 | 296 |
| 1499 | Methyl linolenate | 2036 | C19H32O2 | 292 |
| 1500 | Methyl linoleate | 2046 | C19H34O2 | 294 |
| 1501 | Methyl palmitate | 1901 | C17H34O2 | 270 |
| 1502 | Methyl pentadecanoate | 1796 | C16H32O2 | 256 |
| 1503 | Methyl myristate | 1700 | C15H30O2 | 242 |
| 1504 | Methyl octanoate | 1100 | C91H8O2 | 158 |
| 1505 | Methyl nonanoate | 1208 | C10H20O2 | 172 |
| 1506 | Methyl decanoate | 1300 | C11H22O2 | 186 |
| 1507 | (3Z,9E)-Isoligustilide | 1824 | C12H14O2 | 190 |
| 1508 | (Z)-3-Butyliden-4,5,6,7- | 1697 | C12H16O2 | 192 |
| | tetrahydrophthalide | | | |
| 1509 | Neophytadiene (Isomer 1) | 1807 | C20H38 | 278 |
| 1510 | Neophytadiene (Isomer 2) | 1830 | C20H38 | 278 |
| 1511 | Neophytadiene (Isomer 3) | 1849 | C20H38 | 278 |
| 1512 | Eudesm-3-ene 6,7-oxide | 1787 | C15H24O | 220 |
| 1513 | Eudesma-3,7(11)-dien-8-one | 1745 | C15H22O | 218 |
| 1514 | 5-Methylfurfural | 941 | C6H6O2 | 110 |
| 1515 | g-Costol | 1732 | C15H24O | 220 |
| 1516 | a-Costol | 1761 | C15H24O | 220 |
| 1517 | b-Costol | 1754 | C15H24O | 220 |
| 1518 | Dehydrocostunolide | 1956 | C15H18O2 | 230 |
| 1519 | Dihydrodehydrocostus lactone | 1903 | C15H20O2 | 232 |
| 1520 | Eudesma-4(15),11-dien-8-one | 1643 | C15H22O | 218 |
| 1521 | (E)-15,16-Bisnorlabda-8(17),12- | 2051 | C18H28O | 260 |
| | dien-14-al | | | |
| 1522 | (E)-15,16-Bisnorlabda-8(17),11- | 1958 | C18H28O | 260 |
| | dien-13-one | | | |
| 1523 | Albicanol | 1736 | C16H28O | 236 |
| 1524 | g-Bicyclofarnesal | 1656 | C15H24O | 220 |
| 1525 | trans-6,6,10-Trimethyl-2-decalone | 1505 | C13H22O | 194 |
| 1526 | Coronarin E | 2095 | C20H28O | 284 |
| 1527 | 10-Hydroxy-4-oplopanone | 1708 | C15H26O2 | 238 |
| 1528 | Valerenic acid | 1843 | C15H22O2 | 234 |
| 1529 | Vulgarone A | 1580 | C15H22O | 218 |
| 1530 | Artemisiatriene | 923 | C10H16 | 136 |
| 1531 | 2-Methylbutyl octanoate | 1427 | C13H26O2 | 214 |
| 1532 | Hexyl hexanoate | 1363 | C12H24O2 | 200 |
| 1533 | (E)-2-Decenal | 1240 | C10H18O | 154 |
| 1534 | 2-methylbutyl hexanoate | 1235 | C11H22O2 | 186 |
| 1535 | n-Hexyl 2-methylbutanoate | 1220 | C11H22O2 | 186 |
| 1536 | n-Hexyl butanoate | 1176 | C10H20O2 | 172 |
| 1537 | (E)-2-Heptenal | 942 | C7H12O | 112 |
| 1538 | Fenchyl acetate (Isomer) | 1224 | C12H20O2 | 196 |
| 1539 | 11-Nordrim-8-en-12-al | 1609 | C14H22O | 206 |
| 1540 | Undecanoic acid | 1452 | C11H22O2 | 186 |
| 1541 | Allyl-2,4-di-acetoxybenzene | 1592 | C13H14O4 | 234 |
| 1542 | n-Decane | 993 | C10H22 | 142 |
| 1543 | n-Dodecane | 1200 | C12H26 | 170 |
| 1544 | n-Tridecane | 1300 | C13H28 | 184 |
| 1545 | n-Tetradecane | 1392 | C14H30 | 198 |
| 1546 | n-Pentadecane | 1500 | C15H32 | 212 |
| 1547 | n-Hexadecane | 1600 | C16H34 | 226 |
| 1548 | n-Heptadecane | 1700 | C17H36 | 240 |
| 1549 | n-Octadecane | 1792 | C18H38 | 254 |
| 1550 | n-Eicosane (C-20) | 2000 | C20H42 | 282 |
| 1551 | n-Heneicosane (C-21) | 2100 | C21H44 | 296 |
| 1552 | n-Docosane (C-22) | 2200 | C22H46 | 310 |
| 1553 | n-Tricosane (C-23) | 2301 | C23H48 | 324 |
| 1554 | n-Tetracosane (C-24) | 2400 | C24H50 | 338 |
| 1555 | n-Pentacosane (C-25) | 2498 | C25H52 | 352 |
| 1556 | n-Hexacosane (C-26) | 2598 | C26H54 | 366 |
| 1557 | Verbenene | 982 | C10H14 | 134 |
| 1558 | trans-Chrysanthenyl acetate | 1214 | C12H18O2 | 194 |
| 1559 | trans-Chrysanthenol | 1096 | C10H16O | 152 |
| 1560 | (Z)-2,6-Dimethylocta-1,5,7-trien-3- | 1048 | C10H16O | 152 |
| | ol | | | |
| 1561 | (E)-2,6-Dimethylocta-1,5,7-trien-3- | 1058 | C10H16O | 152 |
| | ol | | | |
| 1562 | Dihydrodiplophylline | 1896 | C15H22O2 | 234 |
| 1563 | Diplophylline | 1965 | C15H20O2 | 232 |
| 1564 | Ginsensene | 1353 | C15H24 | 204 |
| 1565 | 2-Methyl-2,5-divinyltetrahydrofuran | 900 | C9H14O | 138 |
| 1566 | 5-Ethyl-2-methyl-2- | 893 | C9H16O | 140 |
| | vinyltetrahydrofuran | | | |
| 1567 | (all-E)-1,7-Dimethylcyclodeca- | 1274 | C12H18 | 162 |
| | 1,4,7-triene | | | |
| 1568 | Salviadienol | 1545 | C15H24O | 220 |
| 1569 | Torilenol | 1599 | C13H20O | 192 |
| 1570 | Betaer-13-ene | 2040 | C20H32 | 272 |
| 1571 | Ethylbenzene | 843 | C8H10 | 106 |
| 1572 | 4-epi-11-Noraristola-9,11-diene | 1399 | C14H20 | 188 |
| 1573 | 4-epi-11-Noraristola-1,9,11-triene | 1419 | C14H18 | 186 |
| 1574 | 4-epi-11-Noraristola-1(10),11-diene | 1409 | C14H20 | 188 |
| 1575 | 4-Ethylguiacol | 1257 | C9H12O2 | 152 |
| 1576 | 2-Hydroxy-4-methoxyacetophenone | 1294 | C9H10O3 | 166 |
| 1577 | 4-Vinylanisol | 1134 | C9H10O | 134 |
| 1578 | p-Ethylanisol | 1099 | C9H12O | 136 |
| 1579 | 1-Acetoxy-4-ethylbenzene | 1238 | C10H12O2 | 164 |
| 1580 | Tetradecanal | 1596 | C14H28O | 212 |
| 1581 | 4-Ethylphenol | 1139 | C8H10O | 122 |
| 1582 | Dehydropinguisenol | 1800 | C15H20O2 | 232 |
| 1583 | Fusicocca-2,5-diene | 2020 | C20H32 | 272 |
| 1584 | Crispatanolide | 1760 | C15H22O2 | 234 |
| 1585 | (+)-Himachala-2,4-diene | 1433 | C15H24 | 204 |
| 1586 | Polygodial | 1839 | C15H22O2 | 234 |
| 1587 | 9-epi-Polygodial | 1960 | C15H22O2 | 234 |
| 1588 | Dihydrofrullanolide | 1874 | C15H22O2 | 234 |
| 1589 | Eudesma-3,11-dien-8-one | 1666 | C15H22O | 218 |
| 1590 | 5,8a-Dimethyl-3,4,4a,7,8,8a- | 1464 | C12H18O | 178 |
| | hexahydro-1H-naphthalen-2-one | | | |
| 1591 | 8a-Hydroxyeudesma-3,11-diene | 1668 | C15H24O | 220 |
| 1592 | 6,11-Epoxyeudesmane | 1521 | C15H26O | 222 |
| 1593 | Eudesma-5,7(11)-diene | 1543 | C15H24 | 204 |
| 1594 | 6b-Hydroxyeudesm-11-ene | 1643 | C15H26O | 222 |
| 1595 | 6a-Hydroxyeudesm-11-ene | 1598 | C15H26O | 222 |
| 1596 | 6,7-seco-Eudesm-7(11)-en-6-al | 1615 | C15H26O | 222 |
| 1597 | Ipsenol | 1083 | C10H18O | 154 |
| 1598 | Phytane | 1808 | C20H42 | 282 |
| 1599 | Farnesane | 1375 | C15H32 | 212 |
| 1600 | b-n-Octyl-g-butanolide | 1655 | C12H22O2 | 198 |
| 1601 | Crocetane | 1810 | C20H42 | 282 |
| 1602 | Pristane | 1706 | C19H40 | 268 |
| 1603 | cis-Eudesma-4,11-dien-8-ol | 1648 | C15H24O | 220 |
| 1604 | Bisabola-1(6),2,10Z-trien-12-al | 1733 | C15H22O | 218 |
| 1605 | 8,9-Epoxyselina-4,11-diene | 1597 | C15H22O | 218 |
| 1606 | Eudesma-4(15),11-dien-5-ol | 1629 | C15H24O | 220 |
| 1607 | cis-Eudesma-4(15),11-dien-5-ol | 1623 | C15H24O | 220 |
| 1608 | Pentadecanal | 1702 | C15H30O | 226 |
| 1609 | 3-Methylbutanolide | 909 | C5H8O2 | 100 |
| 1610 | 2-n-Propyl-g-butanolide | 1100 | C7H12O2 | 128 |
| 1611 | 2-n-Pentyl-g-butanolide | 1311 | C9H16O2 | 156 |
| 1612 | 2-Ethylbutanolide | 1000 | C6H10O2 | 114 |
| 1613 | 2-Methylbutanolide | 902 | C5H8O2 | 100 |
| 1614 | 4-Allyl-g-butanolide | 1090 | C7H10O2 | 126 |
| 1615 | d-Tetradecalactone | 1893 | C14H26O2 | 226 |
| 1616 | d-Tridecanolide | 1786 | C13H24O2 | 212 |
| 1617 | d-Dodecanolide | 1675 | C12H22O2 | 198 |
| 1618 | g-n-Tetradecyl-g-butanolide | 2720 | C18H34O2 | 282 |
| 1619 | d-Hexaolide | 1044 | C6H10O2 | 114 |
| 1620 | N-2-[(4-Hydroxyphenyl)-ethyl]- | 2325 | C13H17O2N | 219 |
| | tiglamide | | | |
| 1621 | 4-Hydroxy-b-ionone | 1628 | C13H20O2 | 208 |
| 1622 | (E)-Megastigm-7-en-3,9-dione (t) | 1572 | C13H20O2 | 208 |
| 1623 | a-Helmiscapene | 1447 | C15H24 | 204 |
| 1624 | Methyl 2-(2-methylbutyroxy)-3- | 1339 | C12H22O4 | 230 |
| | methylpentanoate | | | |
| 1625 | 7,8-Dihydro-b-ionol | 1431 | C13H24O | 196 |
| 1626 | Dodecyl acetate | 1585 | C14H28O2 | 228 |
| 1627 | (Z)-Heptadec-8-ene | 1666 | C17H34 | 238 |
| 1628 | cis-Dracunculifolione | 1500 | C15H24O | 220 |
| 1629 | Italicen-13-ol | 1670 | C15H24O | 220 |
| 1630 | 10-epi-cis-Dracunculifoliol | 1533 | C15H26O | 222 |
| 1631 | cis-Dracunculifoliol | 1534 | C15H26O | 222 |
| 1632 | trans-Dracunculifoliol | 1581 | C15H26O | 222 |
| 1633 | 3-Hydroxy-b-ionone | 1647 | C13H20O2 | 208 |
| 1634 | 3-Hydroxy-5,6-dihydro-b-ionone | 1609 | C13H22O2 | 210 |
| 1635 | (E)-b-Santalol | 1680 | C15H24O | 220 |
| 1636 | o-Cymene | 976 | C10H14 | 134 |
| 1637 | Methyl 11-methyltridecanoate | 1668 | C15H30O2 | 242 |
| 1638 | Libocedrol | 2326 | C22H30O4 | 358 |
| 1639 | Aristol-1(10)-en-12-ol | 1712 | C15H24O | 220 |
| 1640 | Costunolide | 1914 | C15H20O2 | 232 |
| 1641 | 7-Hydroxyeudesm-4-en-6-one | 1703 | C15H24O2 | 236 |
| 1642 | Aristol-1(10)-en-12-al | 1704 | C15H22O | 218 |
| 1643 | Methyl 10-methyldodecanoate | 1575 | C14H28O2 | 228 |
| 1644 | Dotriacontane | 3200 | C32H66 | 450 |
| 1645 | Eudesma-4(15),7(11),9-trien-12- | 1971 | C15H18O2 | 230 |
| | olide | | | |
| 1646 | Isogermafurenolide | 1867 | C15H20O2 | 232 |
| 1647 | Chloranthalactone A | 1941 | C15H16O2 | 228 |
| 1648 | Ethyl decanoate | 1375 | C12H24O2 | 200 |
| 1649 | Ethyl palmitate | 1954 | C18H36O2 | 284 |
| 1650 | 7,11-Epoxymegastigm-5-en-9-one | 1551 | C13H20O2 | 208 |
| 1651 | Neoiso-isopulegol | 1164 | C10H18O | 154 |
| 1652 | b-Ionol | 1400 | C13H22O | 194 |
| 1653 | 8-Hydroxylinalyl tiglate | 1760 | C15H24O3 | 252 |
| 1654 | (Z)-Methyl 4-(geranyloxy)- | 2334 | C20H26O3 | 314 |
| | cinnamate | | | |
| 1655 | (E)-Methyl 4-(geranyloxy)- | 2461 | C20H26O3 | 314 |
| | cinnamate | | | |
| 1656 | Tetradecyl acetate | 1775 | C16H32O2 | 256 |
| 1657 | Benzyl 3-methylbutyrate | 1366 | C12H16O2 | 192 |
| 1658 | Benzyl 2-methylbutyrate | 1360 | C12H16O2 | 192 |
| 1659 | Decanoic acid | 1347 | C10H20O2 | 172 |
| 1660 | n-Octanoic acid | 1156 | C8H16O2 | 144 |
| 1661 | Dihydromayurone | 1591 | C14H22O | 206 |
| 1662 | Ethyl hexadecanoate | 1990 | C18H36O2 | 284 |
| 1663 | 8-Hydroxylinalyl propionate | 1551 | C13H22O3 | 226 |
| 1664 | 4-Acetoxy-3-methoxyacetophenone | 1434 | C11H12O4 | 208 |
| 1665 | o-Anisaldehyde | 1202 | C8H8O2 | 136 |
| 1666 | 2-Octanone | 964 | C8H16O | 128 |
| 1667 | (E)-trans-Bergamotol | 1680 | C15H24O | 220 |
| 1668 | Methyl 3-methylpentanoate | 853 | C7H14O2 | 130 |
| 1669 | Methyl 3,7-dimethyloctanoate | 1207 | C11H22O2 | 186 |
| 1670 | Methyl 4-methylhexanoate | 974 | C8H16O2 | 144 |
| 1671 | Muurolan-4,7-oxide | 1480 | C15H26O | 222 |
| 1672 | cis-Totarol | 2252 | C20H30O | 286 |
| 1673 | 4-Butyl-3-methyl-g-butanolide | 1252 | C9H16O2 | 156 |
| 1674 | Isosaccogynol | 1740 | C15H22O | 218 |
| 1675 | Isosaccogynone | 1744 | C15H20O | 216 |
| 1676 | Taylorione | 1617 | C15H22O | 218 |
| 1677 | 2-a-Acetoxy-11-methoxyamorpha- | 1846 | C18H28O3 | 292 |
| | 4,7-diene | | | |
| 1678 | 2-a-Acetoxyamorpha-4,7(11)-dien- | 1963 | C17H24O3 | 276 |
| | 8-one | | | |
| 1679 | Neryl formate | 1265 | C11H18O2 | 182 |
| 1680 | Geranyl butyrate | 1534 | C15H26O2 | 238 |
| 1681 | Nerylpropionate | 1451 | C14H24O2 | 224 |
| 1682 | Geranyl tiglate | 1670 | C15H24O2 | 236 |
| 1683 | 2-Methylbutyl angelate | 1130 | C10H18O2 | 170 |
| 1684 | 3-Methylbutyl angelate | 1125 | C10H18O2 | 170 |
| 1685 | Methallyl angelate | 1040 | C9H14O2 | 154 |
| 1686 | 3-Methylbutyl methacrylate | 1018 | C9H16O2 | 156 |
| 1687 | 3-Methylbutyl isobutyrate | 994 | C9H18O2 | 158 |
| 1688 | 3-Methylpentyl isobutyrate | 1095 | C10H20O2 | 172 |
| 1689 | 3-Methylpentyl angelate | 1230 | C11H20O2 | 184 |
| 1690 | Butyl angelate | 1065 | C9H16O2 | 156 |
| 1691 | 10-Acetoxy-4-oplopanone | 1874 | C17H28O3 | 280 |
| 1692 | Butyl benzoate | 1556 | C11H14O2 | 178 |
| 1693 | Propyl benzoate | 1347 | C10H12O2 | 164 |
| 1694 | Phenylacetonitrile | 1085 | C8H7N | 117 |
| 1695 | Hexadecyl acetate | 1847 | C18H36O2 | 284 |
| 1696 | Octadecyl acetate | 2084 | C20H40O2 | 312 |
| 1697 | Octadecanal | 2012 | C18H36O | 268 |
| 1698 | Hexadecanal | 1782 | C16H32O | 240 |
| 1699 | Heptacosane | 2700 | C27H56 | 380 |
| 1700 | Docosanal | 2338 | C22H44O | 324 |
| 1701 | cis-b-Elemene | 1381 | C15H24 | 204 |
| 1702 | Eicosanal | 2170 | C20H40O | 296 |
| 1703 | 1-Methyl-3-(2-oxopropyl)-4-(1- | 1409 | C13H20O | 192 |
| | methylethenyl)-cyclohexene | | | |
| 1704 | Ginsenol | 1621 | C15H26O | 222 |
| 1705 | 4-n-Propylanisol | 1254 | C10H14O | 150 |
| 1706 | n-Decyl acetate | 1390 | C12H24O2 | 200 |
| 1707 | Dimethyl-tetrasulfide | 1181 | C2H6S4 | 158 |
| 1708 | Dimethyl trisulfide | 942 | C2H6S3 | 126 |
| 1709 | S,S-Dimethyl dithiocarbonate | 935 | C3H6OS2 | 122 |
| 1710 | 2,5-Diethyltetrahydrofuran | 875 | C8H16O | 128 |
| 1711 | Neoiso-isopulegol acetate | 1366 | C12H20O2 | 196 |
| 1712 | Methyl 2-hydroxy-4-methoxy-6- | 1555 | C10H12O4 | 196 |
| | methylbenzoate | | | |
| 1713 | 1-Octen-3-yl 3-methylbutyrate | 1315 | C13H24O2 | 212 |
| 1714 | 1-Octen-3-yl 2-methylbutyrate | 1310 | C13H24O2 | 212 |
| 1715 | Oct-1-en-3-yl butyrate | 1266 | C12H22O2 | 198 |
| 1716 | 1-Octen-3-yl isobutyrate | 1223 | C12H22O2 | 198 |
| 1717 | l-Octen-3-yl propanoate | 1181 | C11H20O2 | 184 |
| 1718 | 14-Hydroxy-4,5-dihydro-b- | 1692 | C15H26O | 222 |
| | caryophyllene | | | |
| 1719 | 14-Hydroxy-b-caryophyllene | 1656 | C15H24O | 220 |
| 1720 | 4,5-Dihydro-b-caryophyllen-14-al | 1610 | C15H24O | 220 |
| | (Isomer 1) | | | |
| 1721 | 4,5-Dihydro-b-caryophyllen-14-al | 1621 | C15H24O | 220 |
| | (Isomer 2) | | | |
| 1722 | 4-Desmethylcaryophyll-8(14)-en-5- | 1521 | C14H22O | 206 |
| | one | | | |
| 1723 | Isocaryophyllen-14-al (b-Betulenal) | 1630 | C15H22O | 218 |
| 1724 | 1-Angeloyloxyverbenone | 1694 | C15H20O3 | 248 |
| 1725 | 4-Hydroxy-2-methylacetophenone | 1254 | C9H10O2 | 150 |
| 1726 | 7-epi-1,2-Dehydrosesquicineole | 1460 | C15H24O | 220 |
| 1727 | 1,2-Dimethoxybenzene (Veratrol) | 1117 | C8H10O2 | 138 |
| 1728 | (E)-Isoelemicin | 1614 | C12H16O3 | 208 |
| 1729 | (Z)-Isoelemicin | 1559 | C12H16O3 | 208 |
| 1730 | 1,2,4-Trimethoxybenzene | 1330 | C9H12O3 | 168 |
| 1731 | p-Menth-1-en-9-al (Isomer 1) | 1188 | C10H16O | 152 |
| 1732 | p-Menth-1-en-9-al (Isomer 2) | 1190 | C10H16O | 152 |
| 1733 | (Methoxymethyl)-benzene | 964 | C8H10O | 122 |
| 1734 | 1,4-Dimethoxybenzene | 1132 | C8H10O2 | 138 |
| 1735 | 6,10,14-Trimethylpentadecan-2-one | 1817 | C18H36O | 268 |
| 1736 | (Z)-a-Damascone | 1343 | C13H20O | 192 |
| 1737 | Lilac alcohol (2R,2′R,5′S) | 1210 | C10H18O2 | 170 |
| 1738 | Lilac alcohol (2R,2′S,5′S)) | 1196 | C10H18O2 | 170 |
| 1739 | Lilac alcohol (2S,2′S,5′S) | 1185 | C10H18O2 | 170 |
| 1740 | Lilac aldehyde (2R,2′R,5′S) | 1146 | C10H16O2 | 168 |
| 1741 | Lilac aldehyde (2R,2′S,5′S) | 1133 | C10H16O2 | 168 |
| 1742 | Lilac aldehyde (2S,2′S,5′S) | 1124 | C10H16O2 | 168 |
| 1743 | Methyl citronellate | 1245 | C11H20O2 | 184 |
| 1744 | Myli-4(15)-ene | 1418 | C15H22 | 202 |
| 1745 | Maali-4(15)-en-1-ol | 1624 | C15H24O | 220 |
| 1746 | (E)-Taylopyran | 1530 | C15H22O | 218 |
| 1747 | 7-epi-Bourbon-3-ene 5,11-oxide | 1473 | C15H22O | 218 |
| 1748 | Mylian-3-one | 1593 | C15H22O | 218 |
| 1749 | Myli-4(15)-en-3-one | 1610 | C15H20O | 216 |
| 1750 | 5,5-Dimethyl-1-vinylbicyclo- | 931 | C10H16 | 136 |
| | [2.1.1] hexane | | | |
| 1751 | Cara-2,4-diene | 900 | C10H14 | 134 |
| 1752 | Eudesm-4(15)en-6-one | 1616 | C15H24O | 220 |
| 1753 | Eudesm-4-en-6-one | 1605 | C15H24O | 220 |
| 1754 | Guaia-3,9-diene 5,11-oxide | 1519 | C15H22O | 218 |
| 1755 | Guaia-3,10(14)-diene 5,11-oxide | 1555 | C15H22O | 218 |
| 1756 | 3-Ethylacetophenone | 1260 | C10H12O | 148 |
| 1757 | 4-Ethylacetophenone | 1240 | C10H12O | 148 |
| 1758 | (6Z,8E)-Megastigma-4,6,8-trien-3- | 1553 | C13H18O | 190 |
| | one | | | |
| 1759 | (E,E)-Megastigma-4,6,8-trien-3-one | 1598 | C13H18O | 190 |
| 1760 | Aromadendra-1(10),4(15)-diene | 1506 | C15H22 | 202 |
| 1761 | Perfora-1,7-diene | 1543 | C15H24 | 204 |
| 1762 | Guaia-1(10),11-diene | 1516 | C15H24 | 204 |
| 1763 | Guaia-9,11-diene | 1522 | C15H24 | 204 |
| 1764 | Norpinguisone | 1600 | C14H18O2 | 218 |
| 1765 | Methyl norpinguisonate | 1776 | C15H18O4 | 262 |
| 1766 | Bisabola-1,3,5,7(14)-tetraene | 1484 | C15H22 | 202 |
| 1767 | Lemnalone | 1611 | C15H22O | 218 |
| 1768 | Methyl 2,4-Dimethoxy-6- | 1588 | C11H14O4 | 210 |
| | methylbenzoate | | | |
| 1769 | Methyl 6-Methoxy-2-methyl-3,4- | 1661 | C11H12O5 | 224 |
| | methylendioxybenzoate | | | |
| 1770 | Methyl 6-Hydroxy-2-methyl-3,4- | 1640 | C10H10O5 | 210 |
| | methylendioxybenzoate | | | |
| 1771 | Methyl 3,4,6-trimethoxy-2- | 1705 | C12H16O5 | 240 |
| | methylbenzoate | | | |
| 1772 | 4-Methoxyphenylacetaldehyde | 1255 | C9H10O2 | 150 |
| 1773 | Aromadendra-4,9-diene | 1534 | C15H22 | 202 |
| 1774 | Aromadendra-1(10),4-diene | 1462 | C15H22 | 202 |
| 1775 | Aromadendra-4,10(14)-diene | 1440 | C15H22 | 202 |
| 1776 | 5,6-Dihydro-1,4-dimethylazulene | 1428 | C12H14 | 158 |
| 1777 | 3,4,5,6-Tetrahydro-1,4- | 1246 | C12H16 | 160 |
| | dimethylazulene | | | |
| 1778 | 2,3,3a,4,5,6-Hexahydro-1,4- | 1447 | C12H18O | 178 |
| | dimethylazulen-3-ol | | | |
| 1779 | 3a-Acetoxyamorpha-4,7(11)-diene | 1780 | C17H26O2 | 262 |
| 1780 | Amorpha-2,4,7(11)-triene | 1449 | C15H22 | 202 |
| 1781 | Amorpha-4,7(11)-dien-2-one | 1645 | C15H22O | 218 |
| 1782 | 2a-Acetoxyamorpha-4,7(11)-diene | 1796 | C17H26O2 | 262 |
| 1783 | 2b-Acetoxyamorpha-4,7(11)-diene | 1722 | C17H26O2 | 262 |
| 1784 | 9a-Hydroxyamorpha-4,7(11)-diene | 1680 | C15H24O | 220 |
| 1785 | 7b-Hydroxyamorpha-4,11-diene | 1615 | C15H24O | 220 |
| 1786 | 3a-Hydroxyamorpha-4,7(11)-diene | 1665 | C15H24O | 220 |
| 1787 | Amorpha-4,7(11)-dien-3-one | 1677 | C15H22O | 218 |
| 1788 | Eudesma-4,11-dien-9-one | 1649 | C15H22O | 218 |
| 1789 | 2,8-Epoxyamorpha-4,7(11)-diene | 1597 | C15H22O | 218 |
| 1790 | 5,9-Epoxyamorpha-3,7(11)-diene | 1594 | C15H22O | 218 |
| 1791 | n-Tridecanal | 1493 | C13H26O | 198 |
| 1792 | (E)-Non-2-en-4-one 306 | 1098 | C9H16O | 140 |
| 1793 | (E)-3-Methylnon-2-en-4-one | 1190 | C10H18O | 154 |
| 1794 | Isotheaspirane (Isomer 1) | 1263 | C13H22O | 194 |
| 1795 | Isotheaspirane (Isomer 1) | 1279 | C13H22O | 194 |
| 1796 | Chiloscyphone | 1576 | C15H22O | 218 |
| 1797 | 2-Hydroxy-3,5-dimethoxy-9,10- | 2251 | C16H16O3 | 256 |
| | dihydrophenanthrene | | | |
| 1798 | 4,5-Dihydroxy-3-methoxy-9,10- | 2330 | C15H14O3 | 242 |
| | dihydrophenanthrene | | | |
| 1799 | Isozierene | 1556 | C15H22 | 202 |
| 1800 | Isogermacrene A | 1502 | C15H24 | 204 |
| 1801 | Iso-b-elemene | 1359 | C15H24 | 204 |
| 1802 | n-Decyl butanoate | 1567 | C14H28O2 | 228 |
| 1803 | g-Palmitolactone | 2081 | C16H30O2 | 254 |
| 1804 | n-Octyl butanoate | 1371 | C12H24O2 | 200 |
| 1805 | Benzyl butanoate | 1313 | C11H14O2 | 178 |
| 1806 | n-Butyl butyrate | 970 | C8H16O2 | 144 |
| 1807 | n-Heptyl butanoate | 1270 | C11H22O2 | 186 |
| 1808 | 2-Phenylethyl butyrate | 1412 | C12H16O2 | 192 |
| 1809 | Ethyl 2-phenylhexanoate | 1617 | C14H20O2 | 220 |
| 1810 | Methyl 4-hydroxybenzoate | 1414 | C8H8O3 | 152 |
| 1811 | n-Propyl 4-hydroxybenzoate | 1584 | C10H12O3 | 180 |
| 1812 | n-Octyl hexanoate | 1567 | C14H28O2 | 228 |
| 1813 | 11-b-Hydroxykauren-15-a-yl acetate | 2459 | C22H34O3 | 346 |
| 1814 | a-Campholenic acid | 1304 | C10H16O2 | 168 |
| 1815 | 5-Acetoxybornan-2-one | 1399 | C12H18O3 | 210 |
| 1816 | n-Heptadecanal | 1908 | C17H34O | 254 |
| 1817 | Ventricos-7(13)-ene | 1357 | C15H24 | 204 |
| 1818 | Helminthogermacrene | 1503 | C15H24 | 204 |
| 1819 | allo-Aromadendra-4(15),10(14)- | 1457 | C15H22 | 202 |
| | diene | | | |
| 1820 | Methyl 3-phenylpropanoate | 1242 | C10H12O2 | 164 |
| 1821 | Nepetalactone (Isomer 1) | 1331 | C10H14O2 | 166 |
| 1822 | (all-E)-Geranylcitronellol | 2160 | C20H36O | 292 |
| 1823 | Cyclooctatetraene | 837 | C8H8 | 104 |
| 1824 | 4-(4-Hydroxyphenyl)-butan-2-ol | 1518 | C10H14O2 | 166 |
| 1825 | Lowry's phenol | 1684 | C12H16O4 | 224 |
| 1826 | Platyphyllol | 1588 | C12H16O4 | 224 |
| 1827 | Eugenitine | 1944 | C12H12O4 | 220 |
| 1828 | Isoeugenitine | 1963 | C12H12O4 | 220 |
| 1829 | Dihydrocolumellarine | 1889 | C15H22O2 | 234 |
| 1830 | Myltaylenol | 1727 | C15H24O | 220 |
| 1831 | a-Gorgonene | 1490 | C15H24 | 204 |
| 1832 | Aromadendra-4(15),10(14)dien-1- | 1579 | C15H22O | 218 |
| | ol | | | |
| 1833 | 10-epi-Dihydroagarofuran | 1520 | C15H26O | 222 |
| 1834 | 3,7-Dimethyl-3,7-dihydroxyoct-1- | 1198 | C10H20O2 | 172 |
| | ene | | | |
| 1835 | Agarospirol | 1635 | C15H26O | 222 |
| 1836 | 10a-Hydroxy-12-prenylguai-11-ene | 2111 | C20H34O | 290 |
| 1837 | 5-Formyl-2-hydroxy-(3- | 1617 | C12H14O3 | 206 |
| | methylbutyro)-phenone | | | |
| 1838 | 4-Hydroxybenzaldehyde | 1316 | C7H6O2 | 122 |
| 1839 | 1,3,5-Trimethyl-1,3,5-triazin-2,4,6- | 1327 | C6H9O3N3 | 171 |
| | trione | | | |
| 1840 | 5-Formyl-2-hydroxy-(3-hydroxy-3- | 1660 | C12H14O4 | 222 |
| | methylbutyro)-phenone | | | |
| 1841 | Octadecanoic acid | 2182 | C18H36O2 | 284 |
| 1842 | Longipinanol, high temp. | 1563 | C15H26O | 222 |
| 1843 | Artemiseol | 970 | C10H16O | 152 |
| 1844 | a-Cyclocitral | 1103 | C10H16O | 152 |
| 1845 | (3E,5Z)-Undeca-1,3,5-triene (Isomer | 1133 | C11H18 | 150 |
| | 2) | | | |
| 1846 | Undeca-1,3,5-triene (Isomer 1) | 1117 | C11H18 | 150 |
| 1847 | 4-(4-Methoxyphenyl)-butan-2-one | 1453 | C11H14O2 | 178 |
| 1848 | Atranol | 1511 | C8H8O3 | 152 |
| 1849 | Chloroatranol | 1466 | C8H703Cl | 186 |
| 1850 | Myrtenyl methyl ether | 1145 | C11H18O | 166 |
| 1851 | 8,14-Cedrane oxide | 1536 | C15H24O | 220 |
| 1852 | Camphene hydrate | 1143 | C10H18O | 154 |
| 1853 | 6-Camphenone | 1082 | C10H14O | 150 |
| 1854 | Desmethoxyencecalin | 1617 | C13H14O2 | 202 |
| 1855 | Bisabola-1,3,5,7-tetraene | 1554 | C15H22 | 202 |
| 1856 | Myltayl-4-ene | 1383 | C15H24 | 204 |
| 1857 | Gorgon-11-en-4-ol | 1617 | C15H26O | 222 |
| 1858 | a-Taylorione | 1586 | C15H22O | 218 |
| 1859 | Taylocyclan | 1477 | C15H22O | 218 |
| 1860 | Taynudol | 1709 | C15H22O | 218 |
| 1861 | Taylofuran | 1635 | C15H24O2 | 236 |
| 1862 | 3-Acetoxytaylorione | 1918 | C17H24O3 | 276 |
| 1863 | Copalol | 2265 | C20H34O | 290 |
| 1864 | 3a-Acetoxybicyclogermacrene | 1769 | C17H26O2 | 262 |
| 1865 | Plagiooxide | 1420 | C15H26O | 222 |
| 1866 | Gymnomitr-3(15)-en-5b-ol | 1653 | C15H24O | 220 |
| 1867 | 5b-Acetoxy-gymnomitr-3(15)-ene | 1758 | C17H26O2 | 262 |
| 1868 | 4b,5b-Diacetoxygymnomitr-3(15)- | 1943 | C19H28O4 | 320 |
| | ene | | | |
| 1869 | 15-Acetoxygymnomitr-3-ene | 1797 | C17H26O2 | 262 |
| 1870 | 3,15-b-Epoxy-4b- | 1875 | C17H26O3 | 278 |
| | acetoxygymnomitrane | | | |
| 1871 | 3,15-a-Epoxy-4b- | 1887 | C17H26O3 | 278 |
| | acetoxygymnomitrane | | | |
| 1872 | Iso-a-humulene | 1474 | C15H24 | 204 |
| 1873 | cis-Anethol | 1230 | C10H12O | 148 |
| 1874 | Cadina-4,11-dien-15-al | 1704 | C15H22O | 218 |
| 1875 | Cadina-4,11-dien-15-ol | 1713 | C15H26O | 222 |
| 1876 | a-Barbatenal | 1659 | C15H22O | 218 |
| 1877 | 15-Nor-3-gymnomitrone | 1609 | C14H22O | 206 |
| 1878 | Bergaptene | 2023 | C12H8O4 | 216 |
| 1879 | Peucedanin | 2243 | C15H14O4 | 258 |
| 1880 | syn-Copalol | 2165 | C20H34O | 290 |
| 1881 | Melanene | 1455 | C15H24 | 204 |
| 1882 | Panaxene | 1312 | C15H24 | 204 |
| 1883 | Panaginsene | 1336 | C15H24 | 204 |
| 1884 | Iso-g-bisabolene | 1523 | C15H24 | 204 |
| 1885 | Viscida-4,9,14-triene | 1862 | C20H32 | 272 |
| 1886 | Trichodiene | 1523 | C15H24 | 204 |
| 1887 | 2-Methyl-3-(4-methoxyphenyl)- | 1324 | C11H14O | 162 |
| | prop-2-ene | | | |
| 1888 | Gymnomitr-3(15)-en-12-oic acid | 1790 | C15H22O2 | 234 |
| 1889 | 12-Acetoxygymnomitr-3(15)-ene | 1795 | C17H26O2 | 262 |
| 1890 | Gymnomitr-3(15)-en-12-al | 1627 | C15H22O | 218 |
| 1891 | Scapanol | 1586 | C15H26O | 222 |
| 1892 | Hydroxycitronellal | 1263 | C10H20O2 | 172 |
| 1893 | 2-(2-Ethoxyethoxy)-ethanol | 985 | C6H14O3 | 134 |
| 1894 | Di-(2-hydroxypropyl)-ether | 1003 | C6H14O3 | 134 |
| 1895 | Benzyl propionate | 1231 | C10H12O2 | 164 |
| 1896 | 2-Methyl-3-(4-isopropylphenyl)- | 1433 | C13H18O | 190 |
| | propanal | | | |
| 1897 | (Z)-b-Curcumen-12-ol | 1732 | C15H24O | 120 |
| 1898 | (Z)-b-Phenylethyl cinnamate | 2006 | C17H16O2 | 152 |
| 1899 | (E)-b-Phenylethyl cinnamate | 1123 | C17H16O2 | 252 |
| 1900 | Phenylethyl benzoate | 1815 | C15H14O2 | 126 |
| 1901 | Phenyl ethyl phenylacetate | 1868 | C16H16O2 | 240 |
| 1902 | Phenyl ethyl propionate | 1468 | C11H14O2 | 178 |
| 1903 | 2-(4-Methoxyphenyl)-5-methoxy- | 2237 | C16H16O3 | 256 |
| | 2,3-dihydrobenzo[b]furan | | | |
| 1904 | (Z)-Coriandrin | 1234 | C12H15ONS2 | 153 |
| 1905 | (Z)-Coridrin | 1708 | C10H9ONS | 191 |
| 1906 | (E)-Coridrin | 1784 | C10H9ONS | 191 |
| 1907 | 2-Methylene-6,6-dimethylcyclohex- | 1092 | C10H14O | 150 |
| | 3-ene-1-carbaldehyde | | | |
| 1908 | 1-p-Menthan-8-thiol | 1196 | C10H20S | 172 |
| 1909 | 1-p-Menthen-8-thiol | 1279 | C10H18S | 170 |
| 1910 | 5,5-Dimethylcyclohex-2-en-1,4- | 1002 | C8H10O2 | 138 |
| | dione | | | |
| 1911 | Neoisomenthol | 1176 | C10H20O | 156 |
| 1912 | Menth-2-en-1,4-diol | 1269 | C10H18O2 | 170 |
| 1913 | Carvone hydrate | 1388 | C10H16O2 | 168 |
| 1914 | Carvone hydrate acetate | 1528 | C12H18O3 | 210 |
| 1915 | 8-Hydroxylinalyl isobutyrate | 1588 | C14H24O3 | 240 |
| 1916 | Lyral | 1637 | C13H22O2 | 210 |
| 1917 | Lyral (Isomer) | 1625 | C13H22O2 | 210 |
| 1918 | 2-(4-tert-butylbenzyl)-propione | 1501 | C14H20O | 204 |
| | aldehyde | | | |
| 1919 | Methyl 2-octynoate | 1177 | C9H14O2 | 154 |
| 1920 | Caparapidiol | 1686 | C15H28O2 | 240 |
| 1921 | Jaeschkeanadiol | 1754 | C15H26O2 | 238 |
| 1922 | 2,8-Epithio-cis-p-menthane | 1242 | C10H18S | 170 |
| 1923 | Gorgona-1,4(15),11-triene | 1426 | C15H22 | 202 |
| 1924 | Aromadendra-1(10),3-diene | 1509 | C15H22 | 202 |
| 1925 | 8-Hydroxylinalyl 2-methylbutyrate | 1688 | C15H26O3 | 254 |
| 1926 | trans-Pinane | 951 | C10H18 | 138 |
| 1927 | 4b-Acetoxygymnomitr-3(15)-ene | 1739 | C17H26O2 | 262 |
| 1928 | 1,8-Dimethyl-3-ethyl-2,9- | 1344 | C11H16O3 | 196 |
| | dioxabicyclo[3.3.1]non-7-en-6-one | | | |
| 1929 | 2,6-Diethyl-2,3-dihydro-4H-pyran- | 1221 | C9H14O2 | 154 |
| | 4-one | | | |
| 1930 | Frontaline | 907 | C8H14O2 | 142 |
| 1931 | endo-Brevicomin | 1039 | C9H16O2 | 156 |
| 1932 | cis-Pinane | 963 | C10H18 | 138 |
| 1933 | Chalcograne (Isomer 1) | 1051 | C9H16O | 140 |
| 1934 | Chalcograne (Isomer 2) | 1055 | C9H16O | 140 |
| 1935 | Lineatine | 1102 | C10H16O2 | 168 |
| 1936 | Methyl n-propyl trisufide | 1121 | C4H10S3 | 154 |
| 1937 | (E)n-Propyl 1-propenyl disulfide | 1063 | C6H12S2 | 148 |
| 1938 | Di-n-propyl trisulfide | 1302 | C6H14S3 | 182 |
| 1939 | Methyl n-propyl disulfide | 900 | C4H10S2 | 122 |
| 1940 | Di-n-propyl disulfide | 1081 | C6H14S2 | 150 |
| 1941 | Di-n-propyl tetrasulfide | 1558 | C6H14S4 | 214 |
| 1942 | 5-Pentyl-3,4,5-trimethyl-5H-furan-2- | 1474 | C12H20O2 | 196 |
| | one | | | |
| 1943 | Plagiochilline T | 1665 | C15H20O | 216 |
| 1944 | Plagiochilline U | 1625 | C15H22O | 218 |
| 1945 | 5-Methylcyclohex-2-en-1-one | 935 | C7H10O | 110 |
| 1946 | 3-Ethylcyclohexanone | 1020 | C8H14O | 126 |
| 1947 | Methyl 3-ethyl-4-methylpentanoate | 1021 | C9H18O2 | 158 |
| 1948 | 2,4-Diethyloct-1-ene | 1106 | C12H24 | 168 |
| 1949 | Methyl trans-Dihydrojasmonate | 1623 | C13H22O3 | 226 |
| 1950 | cis-Methyl dihydrojasmonate | 1651 | C13H22O3 | 226 |
| 1951 | 1,7-Dioxaspiro[5.5]undecane | 1108 | C9H16O2 | 156 |
| 1952 | (2Z,4E)-Methyl abscisate | 2076 | C16H22O4 | 278 |
| 1953 | (2Z,4E)-Methyl phaseate | 2141 | C16H22O5 | 294 |
| 1954 | (2E,4E)-Methyl abscisate | 2164 | C16H22O4 | 278 |
| 1955 | Pityol | 945 | C8H16O2 | 144 |
| 1956 | 6-Ethyl-2-methyl-2,3-dihydro-4H- | 1117 | C8H12O2 | 140 |
| | pyran-2-one | | | |
| 1957 | n-Nonyl acetate | 1283 | C11H22O2 | 186 |
| 1958 | 4-epi-Maaliol | 1549 | C15H26O | 222 |
| 1959 | Plagiochiline H | 1807 | C17H24O3 | 276 |
| 1960 | 5-Methyloctahydrofuro [3,2- | 1028 | C9H16O2 | 156 |
| | b]oxepine | | | |
| 1961 | Methyl 2-hydroxyhexanoate | 993 | C7H14O3 | 146 |
| 1962 | Methyl 2-hydroxytetradecanoate | 1838 | C15H30O3 | 258 |
| 1963 | Seudenol | 941 | C7H12O | 112 |
| 1964 | 2,8-Dimethyl-1,7- | 1121 | C11H20O2 | 184 |
| | dioxaspiro[5.5]undecane (Isomer 1) | | | |
| 1965 | 2,8-Dimethyl-1,7- | 1189 | C11H20O2 | 184 |
| | dioxaspiro[5.5]undecane (Isomer 2) | | | |
| 1966 | 4-Methyl-2-buten-4-olide | 869 | C5H6O2 | 98 |
| 1967 | Methyl 2-methyltetradecanoate | 1758 | C16H32O2 | 256 |
| 1968 | Methyl 3-methylpentanoate | 840 | C7H14O2 | 130 |
| 1969 | Methyl 2-methylundecanoate | 1509 | C13H26O2 | 214 |
| 1970 | Methyl 2-methyldodecanoate | 1550 | C14H28O2 | 228 |
| 1971 | Methyl 2-hydroxyisopentanoate | 845 | C6H12O3 | 132 |
| 1972 | Methyl 2-hydroxypentanoate | 894 | C6H12O3 | 132 |
| 1973 | 4a-Methyloctahydronaphthalen-2- | 1369 | C11H18O | 166 |
| | one | | | |
| 1974 | Methyl madelate | 1245 | C9H10O3 | 166 |
| 1975 | Methyl 2-hydroxydodecanoate | 1627 | C13H26O3 | 230 |
| 1976 | 1-Phenylethanol | 1037 | C8H10O | 122 |
| 1977 | 2,3,5-Trimethylvalerolactone | 1158 | C8H14O2 | 142 |
| 1978 | 10-Methyldecalin-2,7-dione | 1520 | C11H16O2 | 180 |
| 1979 | 6-Hexyl-5,6-dihydropyran-2-one | 1551 | C11H18O2 | 182 |
| 1980 | Methyl 2-methylpentadecanoate | 2195 | C17H34O2 | 270 |
| 1981 | 2,3-Epoxycinnamyl alcohol | 1309 | C9H10O2 | 150 |
| 1982 | Methyl 2-methylhexadecanoate | 1972 | C18H36O2 | 284 |

In some embodiments, a composition herein can comprise at least a portion of a plant, an extract thereof or both. In some cases, at least a portion of a plant or an extract thereof can comprise a Chinese herbal remedy. In some instances, a portion of a plant can comprise: a leaf, a stem, a seed, a root, a flower, bark, a wood, a fruit, or any combination thereof. An extract of a plant can comprise an extract of: a leaf, a stem, a seed, a root, a flower, bark, a wood, a fruit, or any combination thereof. In some cases, at least a portion of a plant, an extract thereof, or a tincture thereof, can comprise Boswellia serrata, lemon grass, Borago officinalis, eucalyptus, bromelain, capsaicin, cat's claw, curcumin, turmeric, cinnamon, evening primrose, ginger, garlic, black pepper, willow bark, frankincense, ginkgo, green tea, flaxseed, pine bar, aloe vera, flax, burdock root, nettles, licorice, rose hips, St. John's wort, Devil's claw, hemp, stinging nettle, thyme, thunder god vine, valerian root, or any combination thereof.

In some embodiments, a composition herein can comprise an oil. In some cases, an oil can be an oil of a plant, an animal, or both. In some instances, an oil can be an essential oil. An essential oil can be a plant extract. In some cases, an oil can be a cooking oil. In some cases, an oil can comprise avocado oil, soybean oil, black currant oil, lemon grass oil, peppermint oil, lavender oil, sandalwood oil, bergamot oil, rose oil, chamomile oil, ylang-ylang oil, basil oil, turmeric oil, ginger oil, evening primrose oil, tea tree oil, jasmine oil, rosemary oil, eucalyptus oil, myrrh oil, orange oil, clary sage oil, helichrysum oil, black pepper oil, rose oil, frankincense oil, hemp oil, borage oil or any combination thereof. In some cases, an oil can comprise, fish oil, turtle oil, mink oil, medium-chain triglyceride (MCT) oil, olive oil, vegetable oil, coconut oil, pumpkin seed oil, walnut oil, grapeseed oil, peanut oil, ghee oil, sesame oil, flaxseed oil, sunflower seed oil, canola oil, poppy seed oil, almond oil, apricot kernel oil, jojoba oil, wheat germ oil, safflower oil or any combination thereof.

In some embodiments, a composition herein can comprise a fatty acid. In some cases, a fatty acid can be a short chain fatty acid, a medium chain fatty acid, a long chain fatty acid, an ester of any of these, a salt of any of these, or any combination thereof. A fatty acid can be a saturated fatty acid, an unsaturated fatty acid, a monounsaturated fatty acid, a polyunsaturated fatty acid, an ester of any of these, a salt of any of these, or any combination thereof. In some cases, a fatty acid can comprise an omega-3 fatty acid, an omega-6 fatty acid, an omega-7 fatty acid, an omega-9 fatty acid, an ester of any of these, a salt of any of these, or any combination thereof. For example, an omega-3 fatty acid can comprise alpha-linolenic acid (ALA), eicosapentaenoic acid (EPA), docosahexaenoic acid (DHA), stearidonic acid, docosapentaenoic acid, eicosatrienoic acid, eicosatetraenoic acid, heneicosapentaenoic, heneicosapentaenoic acid, tetracosapentaenoic acid, tetracosahexaenoic acid, an ester of any of these, a salt of any of these or any combination thereof. Omega-6 fatty acids can comprise linoleic acid, gamma-linolenic acid, calendic acid, eicosadienoic acid, dihomo-gamma-linolenic acid, arachidonic acid, docosadienoic acid, adrenic acid, osbond acid, tetracosatetraenoic acid, tetracosapentaenoic acid, an ester of any of these, or a salt of any of these, or any combination thereof. In some cases, Omega-9 fatty acids can comprise hypogeic acid, oleic acid, elaidic acid, gondoic acid, mead acid, erucic acid, nervonic acid, ximenic acid, an ester of any of these, a salt of any of these, or any combination thereof. In some cases, an omega-7 fatty acid can comprise palmitoleic acid, vaccenic acid, rumenic acid, paullinic acid or any combination thereof. In some instances, a fatty acid can be a stearic acid, lauric acid, myristic acid, palmitic acid, a hydroxy fatty acid, a prostaglandin, a derivative or any of these, an ester of any of these, a salt of any of these, or any combination thereof.

A composition described herein can comprise a ceramide, a phospholipid, any combination thereof. In some instances, the phospholipid can be a saturated phospholipid, an unsaturated phospholipid, a monoacylphospholipid or any combination thereof. In some instances, the phospholipid can comprise a liposome. In some instances, the phospholipid can be phosphatidic acid (phosphatidate), phosphatidylethanolamine (cephalin), phosphatidylcholine (lecithin), phosphatidylserine, phosphoinositides, phosphatidylinositol, phosphatidylinositol phosphate, phosphatidylinositol bisphosphate, phosphatidylinositol trisphosphate, ceramide phosphorylcholine (sphingomyelin), ceramide phosphorylethanolamine (sphingomyelin), ceramide phosphoryl lipid, a derivative or any of these, or any combination thereof.

In some embodiments, a composition described herein can comprise a sugar, a sweetening agent or any combination thereof. In some cases, a sugar can comprise granulated sugar, brown sugar, confectioners' sugar, fruit sugar, baker's special sugar, a sugar cube, cane sugar, pearl sugar, or any form of sugar. In some cases, a sugar can comprise a liquid sugar. A liquid sugar can comprise honey, a syrup, a corn syrup (e.g. high fructose corn syrup), glucose syrup, maple syrup, molasses, a nectar (e.g. agave nectar) or any combination thereof. In some cases, sweetening agent can comprise a sugar alcohol (e.g. sorbitol, xylitol, mannitol), aspartame, saccharin, aspartame, acesulfame potassium, sucralose, advantame, neotame, stevia, isomalt maltodextrin or any combination thereof.

In some embodiments, a composition can comprise an inactive ingredient. In some cases an inactive ingredient can comprise beta-caryophyllene, beta-ocimene, caryophyllene oxide, caryophyllene, a terpinolene tincture, terpene oil, humulene, a cannabis plant wax, a nitrogenous compound, an amino acid or salt thereof, an aldehyde, lecithin, flour, chromium picolinate, an herb, a ketone, chocolate, pectin, citric acid, a glycoside, shortening, coffee, coffee beans, a spice, calcium ascorbate, vanilla, manganese proteinate, potassium bicarbonate, egg, carprofen, a pigment, milk, cranberry, a fruit, a berry, a vegetable, a pepper, ethanol, trisodium citrate, a peppercorn, a habanero, a jalapeno, chocolate liquor, cream, glucose sugar syrup, vanilla extract, sea salt, vanilla bean powder, sodium bicarbonate, sorbitol powder, salt, smoked salt, milk fat, milk cream powder, dried skim milk, sunflower lecithin, blueberries, white chocolate, FD&C dye and lake colors, blue 1, blue 2, red 40, yellow 5, yellow 6, green 3, titanium dioxide, Arabic gum, unsweeted chocolate, soy lecithin, butter, water, or any combination thereof. In some embodiments, a composition such as a balm or a lotion can comprise shea butter, cocoa butter, bees wax, mango butter, kokum butter, red cocoa butter, or any combination thereof. In some cases, a composition such as a cream can comprise hemp cream.

In some embodiments, a composition described herein can comprise a flavor. In some cases, a flavor can be a natural flavor, a flavor can be an artificial flavor, or a combination thereof. In some instances, a flavor can comprise acai berry, almond, amaretto, anise, apple, apricot, banana, bacon, bavarian cream, berry cola, birch beer, black licorice, black cherry, black currant, black walnut, blackberry, blue raspberry, blueberry, bourbon, bubble gum, butter, butter almond, butter milk, butter pecan, butter rum, butter toffee, butterscotch, cake batter, candy corn, caramel, caramel apple, caramel cream, carrot cake, champagne, cheesecake, cherry, cherry blossom, cherry cola, cherry cream spice, chocolate, chocolate cake, chocolate mint, cinnamon, cinnamon roll, coconut, coffee, cola, cookie dough, cotton candy, cranberry, cream soda, creme de menthe, cucumber, dill pickle, doughnut, elderberry, egg nog, eucalyptus, flan, fenugreek, fruity cereal, fruit punch, fuzzy navel, ginger ale, ginger beer, ginger lime, gingerbread, gingersnap, grand mariner, graham cracker, grape, grapefruit, green onion, hazelnut, honey, horchata, huckleberry, irish cream, irish whiskey, kettle corn, key lime, kiwi, lemon, lemon bar, lemon custard, lemon lime, lemon meringue pie, lemonade, lime, long island tea, macadamia nut, malted milk, mango, mango orange pineapple, maple, margarita, marshmallow, melon, melon kiwi, mint, mixed berry, mocha, mocha irish cream, mojito, moscow mule, nutmeg, orange, orange brandy, orange cream, orange mandarin, orange mango, orange pineapple, papaya, passion fruit, peach, peanut, pear, pecan, pecan roll, peppermint, pina colada, pineapple, pink lemonade, pistachio, plantation punch, plum, pomegranate, prickly pear, pumpkin, pumpkin pie, punch, quinine, raspberry, raspberry ginger ale, rock and rye, root beer, rum, sarsaparilla, sassafras, smoke, sour, strawberry, strawberry kiwi, spice, spearmint, tangerine, tart lemon, tequila, toasted coconut, toffee, tonic, tropical punch, tutti frutti, vanilla, vanilla cream, watermelon, white chocolate, whipped cream, wild cherry cream, wintergreen, or any combination thereof.

In some embodiments, a composition described herein can comprise a fragrance. In some cases, a fragrance can be a natural fragrance, an artificial fragrance or a combination thereof. In some cases, a fragrance can come from an essential oil or from a plant extract. In some cases, a fragrance can be a spice such as nutmag, can comprise a flavor or both. In some instances, the composition can comprise a fragrance, a paraben, a phthalate, an alcohol, or any combination thereof, for example less than about: 5%, 4%, 3%, 2%, 1%, 0.5%, or 0.1% with respect to a total weight or a total volume of a composition. In some instances, the fragrance can be a perfume, cologne, an aftershave, or any combination thereof. In some instances, the composition can be free from a fragrance, a paraben, a phthalate, an alcohol, or any combination thereof.

In some cases, a composition herein does not comprise a color additive. In some cases, the composition can comprise a color additive. In some cases, the composition may contain an incidental ingredient such as a color additive in an insignificant level in the composition, for example less than about: 5%, 4%, 3%, 2%, 1%, 0.5%, or 0.1% with respect to a total weight or a total volume of a composition. In some cases, the incidental ingredient may have no technical/structural, functional or any combination thereof effect in the composition, e.g., no an active ingredient.

In some cases, a cosmetic composition described herein can include an alpha hydroxy acid, a beta hydroxyl acid, diethanolamine (DEA), a talc or any combination thereof. In some instances, a beta hydroxyl acid can be salicylic acid, beta hydroxybutanoic acid, tropic acid, trethocanic acid, a salt thereof or any combination thereof. In some instances, the diethanolamine can comprise cocamide DEA, cocamide monoethanolamine (MEA), DEA-cetyl phosphate, DEA Oleth-3 phosphate, lauramide DEA, linoleamide MEA, myristamide DEA, oleamide DEA, stearamide MWA, TEA-lauryl sulfate, triethanolamine, a salt thereof, or any combination thereof. In some cases, a composition, such as a cosmetic, can comprise water, glycerol, saline, a flower extract, a mineral oil, a synthetic oil, a silicate, a calcium salt, a magnesium salt potassium chloride, lactic acid, a starch, a sugar alcohol, a cellulose, an activated charcoal, a glycerin, a butter, an amino acid, a paraffin, honey, a wax, an agar, calcium carbonate, a citric acid, tartaric acid, a steric acid, xanthan gum, benzoic acid, a polyethylene glycol, a silicon, derivatives thereof, salts thereof, or any combination thereof. In some cases, a composition disclosed herein can comprise a filler, a binder, a disintegrant, a coating, a sorbent, an anti-adherent, a lubricant, a glidant, an antioxidant, a surfactant, a solvent, a chelating agent, a viscosity imparting agent, a surface active agent a humectant or any combination thereof.

In some cases, a composition disclosed herein can comprise a preservative. In some instances, the preservative can be an organic/natural compound, a synthetic compound or any combination thereof. In some instances, the preservative can be an antimicrobial, an antibacterial, an antifungal, an antiviral, an antiseptic, a detergent, or any combination thereof. In some cases, a cosmetic preservative can be a paraben, a formaldehyde releaser, an isothiazolinone, a phenoxyethanol, an organic acid, a quaternary ammonium compound, or any combination thereof. In some cases, a food preservative can comprise benzoates, nitrites, sulphites, sorbates or any combination thereof.

In some cases, a composition disclosed herein can comprise a cosmetically appropriate ingredient. In some instances, the composition may be safe under labeled or customary conditions of use. In some instances, a packaged product comprising the composition can be properly labeled, and the use of the ingredient does not otherwise cause the cosmetic to be adulterated or misbranded. In some instances, the adulterated condition can include: any poisonous or deleterious substance that can injure a user; a filthy, putrid, or decomposed substance; a cosmetic that may have been prepared, packed or held under insanitary conditions; a container that can comprise poisonous or deleterious substance which may render the contents injurious to health; or any combination thereof.

In some cases, a composition disclosed herein can comprise a dietary supplement appropriate ingredient. In some instances, the composition may be safe under labeled or customary conditions of use. In some instances, a packaged product comprising the composition can be properly labeled, and the use of the ingredient does not otherwise cause the dietary supplement to be adulterated or misbranded. In some instances, the supplement can be manufactured according to good manufacturing practice (cGMP) and labeling regulations.

In some cases, a composition disclosed herein can be aseptic. In some cases, the composition can comprise resident microbes. The microbes can be viruses, bacteria, eukaryotic cells or any combination thereof. In some instances, the microbes may not be pathogenic. In some instances, the composition can comprise a bacterium or bacteria at a concentration of less than about: 10 colony forming units (CFU)/gram (g), 50 CFU/g 100 CFU/g, 150 CFU/g, 200 CFU/g, 300 CFU/g, 400 CFU/g, 500 CFU/g, 600 CFU/g, 700 CFU/g, 800 CFU/g, 900 CFU/g, or 1000 CFU/g. In some cases, the composition can comprise bacteria at a concentration of about: 10 CFU/g to about 1000 CFU/g, 10 CFU/g to about 50 CFU/g, 20 CFU/g to about 100 CFU/g, 50 CFU/g to about 200 CFU/g, 100 CFU/g to about 250 CFU/g, 200 CFU/g to about 500 CFU/g, 500 CFU/g to about 700 CFU/g, or 600 CFU/g to about 1000 CFU/g. In some instances, the composition may be substantially free or free of: Staphylococcus aureus, Streptococcus pyogenes, Pseudomonas aeruginosa, Pseudomonas species, Klebsiella pneumoniae, or any combination thereof.

In some cases, a composition disclosed herein may not contain a heavy metal or toxic ingredients such as lead, bithionol, chlorofluorocarbon propellants, nitrosamines, chloroform, halogenated salicylanilides, hexachlorophene, mercury compounds, 1,4-dioxane, methylene chloride, prohibited cattle materials, sunscreen compounds, vinyl chloride, zirconium-containing complexes, or any combination thereof. In some instances, the prohibited cattle materials can comprise the brain, skull, eyes, trigeminal ganglia, spinal cord, vertebral column, dorsal root ganglia, tonsils, distal ileum of the small intestine or any combination thereof. In some instances, the composition may comprise lead at levels of 10 (parts per million) ppm or less.

In some cases, a kit can comprise a composition disclosed herein. In some cases, a kit can comprise a container. In some instances, a container can be plastic, glass, metal or any combination thereof.

### Methods of Formulating Compositions

In some embodiments, a composition disclosed herein can be formulated as a pharmaceutical composition or a supplement composition. In some cases, a composition can comprise an excipient, a diluent, a carrier or any combination thereof. In some cases, the compositions can be made by mixing a composition described herein, and a pharmaceutically acceptable excipient. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent or any combination thereof. In some instances, the excipient comprising one or more of cellulose, disodium hydrogen phosphate, hydroxypropyl cellulose, hypromellose, lactose, mannitol, or sodium lauryl sulfate. In some instances, the compositions further comprise glyceryl monostearate 40-50, hydroxypropyl cellulose, hypromellose, magnesium stearate, methacrylic acid copolymer type C, polysorbate 80, sugar spheres, talc, or triethyl citrate. In some instances, a composition can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, or yellow ferric oxide. In some instances, a composition can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate. Examples of carriers for the composition include any degradable, partially degradable or non-degradable and generally biocompatible polymer, e.g., polystirex, polypropylene, polyethylene, polacrilex, poly-lactic acid (PLA), polyglycolic acid (PGA) and/or poly-lactic polyglycolic acid (PGLA), e.g., in the form or a liquid, matrix, or bead. In some instances, a binder can comprise starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides or any combination thereof.

In, some embodiments an excipient can comprise a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium gluconate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some embodiments, a pharmaceutical composition can comprise a diluent. Non-limiting examples of diluents can include water, glycerol, methanol, ethanol, and other similar biocompatible diluents. In some cases, a diluent can be an aqueous acid such as acetic acid, citric acid, maleic acid, hydrochloric acid, phosphoric acid, nitric acid, sulfuric acid, or similar. In other cases, a diluent can be selected from a group comprising alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some embodiments, a salt can include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N, N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. In some cases, a salt can comprise a pharmaceutically acceptable salt. In some instances, a salt of a polypeptide or derivative thereof or a compound can be a Zwitterionic salt.

In some embodiments, compositions disclosed herein can be in unit dose forms or multiple dose forms. For example, a pharmaceutical composition or a supplemental composition described herein can be in unit dose form. Unit dose forms, as used herein, refer to physically discrete units suitable for administration to human or non-human subjects (e.g. pets) and packaged individually. Each unit dose can contain a predetermined quantity of an active ingredient(s) that may be sufficient to produce the desired therapeutic effect or to supplement a diet, in association with pharmaceutical carriers, diluents, excipients or any combination thereof. Examples of unit dose forms can include, ampules, syringes, and individually packaged tablets and capsules. In some cases, a unit dose form can be comprised in a food, for example, a chocolate supplement. In some instances, unit-dosage forms can be administered in fractions or multiples thereof. A multiple dose form can be a plurality of identical unit dose forms packaged in a single container, which can be administered in segregated a unit dose form. Examples of a multiple dose form can include vials, bottles of tablets or capsules, bottles of gummies, or bottles of pints or gallons. In some instances, a multiple dose form can comprise different pharmaceutically active agents. In some cases, a unit dose form can be a serving. In some cases, a multiple dose form can have more than about: 1, 2, 3, 4, 5, 6, 7, 8, 9, 10, 15, 20, 30, 40, 50, 60, 70, 80, 90, 100 or 200 servings. In some cases, a multiple dose form can have less than about: 1, 2, 3, 4, 5, 6, 7, 8, 9, 10, 15, 20, 30, 40, 50, 60, 70, 80, 90, 100 or 200 servings. In some cases, a multiple dose form can have from about: 1 serving to about 200 servings, 1 serving to about 20 serving, 5 serving to about 50 servings, 10 servings to about 100 serving, or about 30 serving to about 150 servings.

### Methods of Use

In some embodiments, disclosed herein can be a method, comprising administering a composition disclosed herein to a subject (e.g., a human or a pet) in need thereof. In some instances, the method can treat or prevent a disease in the subject. In some instances, the method can ameliorate a condition of the subject' arthritis. In some instances, a method described herein can comprise administering a composition described herein as a dietary supplement. In some cases, a method described herein can comprise administering a composition herein as a cosmetic.

In some embodiments, disclosed herein can be a method for improving, maintaining, or supplementing joint health. In some instances, the methods can be used to treat arthritis. In some instances, the disease or condition can be chronic arthritis, acute arthritis or both. In some instances, the method can reduce the symptoms of arthritis such as joint stiffness, a tender joint, locking of a joint, a warm joint, a swollen joint, redness of a joint, fatigue, gland swelling, or any combination thereof. In some instances, a method can prevent, reduce or eliminate the severity of an arthritic symptom.

Administration disclosed herein to an area in need of treatment or therapy can be achieved by, for example and not by way of limitation, oral administration, topical administration, intravenous administration, inhalation administration, or any combination thereof. In some cases, delivery can include injection, catheterization, gastrostomy tube administration, intraosseous administration, ocular administration, otic administration, transdermal administration, oral administration, rectal administration, nasal administration, intravaginal administration, intracavernous administration, transurethral administration, sublingual administration, or a combination thereof. Delivery can include direct application to the affect tissue or region of the body. Delivery can include a parenchymal injection, an intrathecal injection, an intra-ventricular injection, or an intra-cisternal injection. A composition provided herein can be administered by any method. A method of administration can be by joint injection, intraarterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump or a combination thereof. In some cases, delivery can be by an enema, an eye drop, a nasal spray, an ear drop, or any combination thereof.

In some cases, administering can be oral ingestion. Oral ingestion delivery can comprise a tea, an elixir, a food, a drink, a syrup, a liquid, a gel, a capsule, a tablet, an oil, a tincture, or any combination thereof. In some cases, a food can be a medical food. In some cases, a medical food can comprise a capsule, a gummy, a candy, a chocolate, a beverage, a food, a liquid, an oil, a syrup or any combination thereof. In some cases, a dietary supplement can be administered by oral injection. In some cases, delivery can include a gummy (e.g. a carrageenan gummy). In some instances, a food can comprise a candy. In some instances, a food can comprise a chocolate. For example, a candy can comprise a gummy, a fruit candy drop, a sea salt caramel, a cookie, a cake, a brownie, a dessert, a caramel, a sucker, a hard candy, or any combination thereof. In some cases, a chocolate can comprise blueberry chocolate, a cranberry chocolate, a chocolate bar, an orange chocolate, a mint chocolate, a raspberry chocolate, a strawberry chocolate, a peanut butter cup, a fruit flavored chocolate, a citrus flavored chocolate, a fruit covered chocolate, a chocolate covered fruit, a pepper covered chocolate (e.g. habanero chocolate), a chocolate caramel, a coffee bean chocolate, a chocolate caramel cup, a chocolate caramel, a chocolate bar flow, a chocolate candy bar, a nut covered chocolate, or any combination thereof. In some cases, a food can be a granola bar, a snack, a cheese, a chip, or any combination thereof. In some cases, a food can comprise a drink, a beverage, a liquid, a syrup, or any combination thereof. In some cases, delivery can be a capsule or a tablet.

In some instances, a capsule can comprise hydroxymethylcellulose. In some cases, a capsule can comprise a gelatin, hydroxypropylmethyl cellulose, pullulan, or any combination thereof. In some cases, capsules can comprise a coating, for example, an enteric coating. In some cases, a capsule can comprise a vegetarian product or a vegan product such as a hypromellose capsule. In some cases, a capsule size can comprise 000, 00, 0, 1, 2, 3, 4, 5, or any size. In some instances, the density of the capsules can be greater than about: 0.1, 0.2, 0.3, 0.4, 0.5, 0.6, 0.7, 0.8, 0.9, or 0.99 grams per milliliter (g/mL). In some cases, the density of the capsules can be less than: 0.1, 0.2, 0.3, 0.4, 0.5, 0.6, 0.7, 0.8, 0.9, or 0.99 g/mL. In some cases, the density of the capsules can be from about 0.01 g/ml to about 0.99 g/mL, from about 0.1 g/ml to about 0.9 g/mL, from about 0.4 g/ml to about 0.7 g/mL or from about 0.8 g/ml to about 0.9 g/mL. In some cases, delivery can comprise inhalation by an inhaler, a diffuser, a nebulizer, a vaporizer or a combination thereof.

In some cases, delivery can include topical application (topical administration). For example, delivery can include topical application of: a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment or any combination thereof. In some cases, topical application can comprise administering to an external surface, such as a skin. In some cases, topical application can comprise applying a cosmetic. In some instances, the composition can be applied by pouring, sprinkling, spraying, rubbing, introduced onto, or otherwise. In some instances, topical administration can be administered directly to the site of the condition.

In some instances, a subject can administer the composition in the absence of supervision. In some cases, a human can administer the composition to the subject. For example, an owner can administer a composition described herein to a pet. In some instances, a subject can administer the composition under the supervision of a medical professional (e.g., a physician, nurse, physician's assistant, orderly, hospice worker, etc.). In some cases, a medical professional can administer the composition. In some cases, a cosmetic professional can administer the composition. In some cases, an animal professional (e.g. a veterinarian, a veterinary assistant, a zookeeper, etc.) can administer the composition.

Administration or application of a composition disclosed herein can be performed for a duration of at least about at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000 days consecutive or nonconsecutive days. In some cases, the composition can be administered for life. In some cases, administration or application of the composition described herein can be from about 1 to about 30 days, from about 1 to about 60 days, from about 1 to about 90 days, from about 1 to about 300 days, from about 1 to about 3000 days, from about 30 day to about 90 days, from about 60 days to about 900 days, from about 30 days to about 900 days, or from about 90 days to about 1500 days. In some cases, administration or application of the composition described herein can be from about: 1 week to about 5 weeks, 1 month to about 12 months, 1 year to about 3 years, 2 years to about 8 years, 3 years to about 10 years, 10 years to about 50 years, 15 years to about 40 years, 25 years to about 100 years, 30 years to about 75 years, 60 years to about 110 years, or about 50 years to about 130 years.

Administration or application of a composition disclosed herein can be performed for a duration of at least about: 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or for life. Administration can be performed repeatedly over a lifetime of a subject, such as once a day, once a week, or once a month for the lifetime of a subject. Administration can be performed repeatedly over a substantial portion of a subject's life, such as once a day, once a week, or once a month for at least about: 1 year, 5 years, 10 years, 15 years, 20 years, 25 years, 30 years, or more.

Administration or application of composition disclosed herein can be performed at least about: 1, 2, 3, 4, 5, 6, 7, 8, 9, 10, 11, 12, 13, 14, 15, 16, 17, 18, 19, 20, 21, 22, 23, or 24 times a in a 24-hour period. In some instances, administration of a capsule, a tablet, a gummy, a chocolate, or a candy can comprise administration of more than one capsules, tablets, gummies, chocolates or candies. In some cases, administration or application of a composition disclosed herein can be performed continuously throughout a 24-hour period. In some cases, administration or application of composition disclosed herein can be performed at least about: 1, 2, 3, 4, 5, 6, 7, 8, 9, 10, 11, 12, 13, 14, 15, 16, 17, 18, 19, 20, or 21 times a week. In some cases, administration or application of a composition disclosed herein can be performed at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or more times a month. In some cases, a composition can be administered as a single dose or as divided doses. In some cases, the compositions described herein can be administered at a first time point and a second time point. In some cases, a composition can be administered such that a first administration can be administered before the other with a difference in administration time of about: 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

In some embodiments, treatment for arthritis can comprise administering a second therapy. A second therapy can comprise acetaminophen, an opioid, a nonsteroidal anti-inflammatory drug, methotrexate, hydroxychloroquine, doxycycline, prednisone, cortisone, a biological response modifier, a salt thereof, or any combination thereof. In some cases, a second therapy can comprise a steroid. A second therapy can comprise a corticosteroid, such as a cortisone joint injection. In some cases, a second therapy can comprise biological response modifier. A biological modified can include abatacept, adalimumab, adalimumab-atto, anakinra, azathioprine, certolizumab pegol, etanercept, etanercept-szzs, hydroxychloroquine, leflunomide, methotrexate, sulfasalazine, tofacitinib, golimumab, infliximab, infliximab-dyyb, rituximab, sarilumab, tocilizumab, a biologically active fragment of any of these, a salt of any of these, or any combination thereof. In some cases, a second therapy can comprise a biosimilar. In some instances, a second therapy can include hyaluronic acid. In some cases, a second therapy can be an immunosuppressive drug, an antibiotic or both. In some cases, a second therapy can comprise a nonsteroidal anti-inflammatory drug (NSAID) such as naproxen, ibuprofen, aspirin, celecoxib, diclofenac, diflunisal, etodolac, indomethacin, ketoprofen, ketorolac, nabumetone, oxaprozin, piroxicam, salsalate, a salt of any of these, or any combination thereof. In some instances, a second therapy can comprise occupational therapy for example, occupational therapy can comprise adding braces or supports to protect one or more joints. In some cases, a second therapy can comprise exercise, altered diet and nutritional intake or any combination thereof. A second therapy can comprise joint aspiration to remove fluid from a joint. In some instances, a second therapy can comprise surgery. For example, a surgery can comprise a synovectomy to remove damaged connective tissue in a joint cavity, a joint replacement (e.g. knee or hip joint) or any combination thereof.

In some embodiments, a subject may have been diagnosed with arthritis. In some cases, a subject may have diagnosed prior to treatment. In some cases, a method described herein can further comprise diagnosing a subject. In some cases, diagnosis may comprise diagnosis with acute arthritis, chronic arthritis, or both. In some cases, diagnosis can comprise a physical examination, a radiological image, a blood test, an antibody test or any combination thereof. A radiological image can comprise a computed tomography (CT) image, an X-Ray image, a magnetic resonance image (MRI), an ultrasound image, a bone scan or any combination thereof. A blood test can comprise measuring rheumatoid factor, anti-cyclic citrullinated peptide, erythrocyte sedimentation rate, C-reactive protein, antinuclear antibody, HLA-B27, complete blood count, creatine kinase, complement, cryoglobulins, or any combination thereof. In some cases, a physical examination can comprise an examination of joints for example, assessing the pain in joints. In some instances, a physical examination can comprise examination of one or more symptoms. In some cases, a diagnosis can comprise measuring a level compared to a standard level. For example, determining the level of rheumatoid factor or determining the level of joint damage on an X-Ray.

### EXAMPLES

### Example 1. Making a Capsule Composition Comprising CBD, Naringenin, Glucosamine Sulfate, Chondroitin Sulfate, and Methylsulfonylmethane

A mixture comprising CBD, naringenin, glucosamine sulfate, chondroitin sulfate, and methylsulfonylmethane (MSM) were contacted together as a dry mix. The composition comprises: 10 mg CBD, 10 mg naringenin, 600 mg glucosamine sulfate, 300 mg chondroitin sulfate, and 375 mg MSM. The composition was added to a 00 size capsule at a density of about 0.8-0.9 g/ml.

### Example 2. Making a Capsule Composition Comprising CBD, Naringenin, Calcium Ascorbate, and MSM

A mixture comprising CBD, naringenin, calcium ascorbate, and methylsulfonylmethane (MSM) were contacted together as a dry mix. The composition comprises: 40 mg CBD, 10 mg naringenin, 500 mg calcium ascorbate, and 750 mg MSM. The composition was added to a 00 size capsule at a density of about 0.85-0.9 g/ml.

### Example 3. Making a Capsule Composition Comprising CBD, Naringenin, Glucosamine Sulfate, and MSM

A mixture comprising CBD, naringenin, glucosamine sulfate, and methylsulfonylmethane (MSM) were contacted together as a dry mix. The composition comprises: 20 mg CBD, 10 mg naringenin, 600 mg glucosamine sulfate, and 700 mg MSM. The composition was added to a 00 size capsule at a density of about 0.85-0.9 g/ml.

### Example 4. Making a Capsule Composition Comprising CBD, Naringenin, Chondroitin Sulfate, and MSM

A mixture comprising CBD, naringenin, chondroitin sulfate, and methylsulfonylmethane (MSM) is contacted together as a dry mix. The composition comprises: 10 mg CBD, 10 mg naringenin, 500 mg chondroitin sulfate, and 750 mg MSM. The composition is added to a 00 size capsule at a density of about 0.85-0.9 g/ml. The composition described is administered as two capsules three times daily to a human subject and to a canine subject. The human subject and the canine subject have increased joint mobility after administration compared to joint mobility prior to treatment.

### Example 5. Making a CBD Balm

A mixture comprising shea butter, cocoa butter, bees wax, MCT oil, lemon grass oil, frankincense oil, eucalyptus oil, naringenin and water-soluble CBD is made into a balm. The composition comprises: 12.37 g shea butter, 12.37 g cocoa butter, 3.32 g bees wax, 12 mL MCT Oil, 0.72 mL lemon grass oil, 0.72 mL frankincense oil 0.72 ml, 0.72 mL eucalyptus oil. 1.0 g of naringenin and 1.5 g CBD. The ingredients are mixed together in large metal pot for 15 to 20 minutes on hot plate at 67 degrees Celsius. Once the ingredients have melted and been mixed for 3 to 5 minutes, the composition is poured into jars. The balm is directly applied to a joint to help improve mobility and decrease swelling.

### Example 6. Making a CBD Tincture

A mixture comprising MCT oil, naringenin and CBD is made into a tincture. The composition comprises: 30 mL MCT Oil, 1.0 g of naringenin, 0.24 mL of mint flavor, and 3 g CBD. The ingredients are mixed together in a 3000 mL beaker. The solution is placed in a double boiler until the CBD is fully emulsified. Then the solution is placed on a hot plate and stirred until the solution is emulsified. The solution is aliquoted into 5 mL oil bottles. The tincture is applied to a finger joint to supplement joint health.

### Example 7. Making a CBD, Naringenin, D-Limonene, and Vitamin C Infused Chocolate Bar Flow Wrap

A mixture comprising liquid CBD, Naringenin, Vitamin C (calcium ascorbate) and D-limonene is added to a recipe for a chocolate bar flow wrap. The chocolate bar flow wrap is a chocolate bar with a caramel center. The chocolate bar flow wrap comprises: 25 mg of CBD, 10 mg of naringenin, 50 mg of Vitamin C, and 125 mcg of D-limonene. The CBD, Naringenin, Vitamin C (calcium ascorbate) and D-limonene are mixed together and added to the dark chocolate bar ingredients (e.g. dark chocolate, cream, glucose syrup, sorbitol powder, sunflower lecithin, cocoa butter, naturally smoked salt). The combined ingredients are mixed completely under low heat until homogenized and poured into a chocolate bar mold that permits caramel to be added once the chocolate mixture is hardened. The chocolate bar flow wrap is ingested by a human subject to supplement the diet and maintain joint health.

### Example 8. Making a CBD, Naringenin, D-Limonene, and Vitamin C Infused Fruit Candy Drop

A mixture comprising liquid CBD, Naringenin, Vitamin C (calcium ascorbate) and D-limonene is added to a recipe for a fruit candy drop. The fruit candy drop is a candy that is sweetened with isomalt. The fruit candy comprises: 10 mg of CBD, 10 mg of naringenin, 100 mg of Vitamin C, and 125 mcg of D-limonene. The CBD, Naringenin, Vitamin C (calcium ascorbate) and D-limonene are mixed together and added to the fruit candy drop ingredients (e.g. isomalt, water, citric acid, flavoring and coloring). The combined ingredients are mixed completely under low heat until homogenized and poured into a fruit candy drop mold. The fruit candy drop is ingested by a pet or a human to supplement the diet and maintain joint health.

### Example 9. Administering a Composition

The composition of example 1 or example 2 is administered as two capsules taken three times daily by a human subject and a canine subject. The human subject and the canine subject have decreased joint inflammation compared to the joint prior to treatment.

What is claimed is:

1. A composition comprising:
a cannabidiol (CBD), a flavanone, or a salt thereof;
and one or more of: a glucosamine, a salt thereof, a glycosaminoglycan, a salt thereof, a vitamin, a derivative thereof, a salt thereof, or methylsulfonylmethane (MSM).

2. The composition of claim 1, wherein the composition is in the form of a capsule, a tablet, a gummy, an oil, a tincture, a lotion, a cream, a balm, a candy, a chocolate, a food, a drink, a liquid, an oil, a syrup, or any combination thereof.

3. The composition of claim 1, wherein the composition further comprises: a monoterpene, a derivative thereof, a salt thereof, or any combination thereof.

4. The composition of claim 1, wherein the composition further comprises: a mineral, a salt thereof, or a chelated form thereof, an amino acid or a salt thereof, at least a portion of a plant or an extract thereof, an oil, a fatty acid, or any combination thereof.

5. The composition of claim 1, wherein the composition further comprises: a sugar, a sucralose, an aspartame, a stevia, or any combination thereof.

6. The composition of claim 1, wherein the composition comprises substantially no Δ9-tetrahydrocannabinol (THC).

7. The composition of claim 1, wherein the composition further comprises Δ8-THC.

8. The composition of claim 1, wherein the composition is a dietary supplement and is in the form of a capsule, a gummy, a candy, a chocolate, a beverage, a food, a liquid, an oil, a syrup or any combination thereof; or is medical food and is in the form of a capsule, a gummy, a candy, a chocolate, a beverage, a food, a liquid, an oil, a syrup, or any combination thereof.

9. The composition of claim 1, wherein the CBD comprises cannabidiol, cannabidiolic acid, or any combination thereof.

10. The composition of claim 1, wherein the CBD is formulated as a powder, an oil, a liquid, or any combination thereof.

11. The composition of claim 10, wherein the CBD is at least partially water soluble.

12. The composition of claim 1, comprising the flavanone or salt thereof which comprises naringenin, a salt thereof, a derivative of naringenin, a salt thereof, naringin, a salt thereof, a derivative of naringin, a salt thereof, or any combination thereof.

13. The composition of claim 12, wherein the flavanone is in the form of an extract from: a grapefruit, a sour orange, a tart cherry, a citrus fruit, a tomato or any combination thereof.

14. The composition of claim 1, comprising the salt of glucosamine which comprises glucosamine sulfate.

15. The composition of claim 1, comprising the salt of glycosaminoglycan which comprises chondroitin sulfate, dermatan sulfate, or any combination thereof.

16. The composition of claim 1, comprising the glycosaminoglycan or salt thereof which comprises hyaluronan or a salt thereof.

17. The composition of claim 1, comprising the salt of glycosaminoglycan which comprises keratan sulfate.

18. The composition of claim 1, comprising the vitamin, the derivative thereof, or the salt thereof which comprises: vitamin A, vitamin C, vitamin D, vitamin E, vitamin K, vitamin B1, vitamin B2, vitamin B3, vitamin B5, vitamin B6, vitamin B7, vitamin B9, vitamin B12, a derivative of any of these, a salt of any of these, or any combination thereof.

19. The composition of claim 18, comprising the vitamin C or salt thereof.

20. The composition of claim 19, comprising the salt of vitamin C which comprises calcium ascorbate.

21. The composition of claim 3, comprising the monoterpene or salt thereof which comprises D-limonene or a salt thereof.

22. The composition of claim 4, comprising the mineral, the chelated form thereof, the salt thereof, or any combination thereof which comprises: a calcium, a phosphorus, a potassium, a sulfur, a sodium, a chloride, a magnesium, an iron, a zinc, a copper, a manganese, an iodine, a selenium, a molybdenum, a chromium, a fluoride, a chelated form thereof, a salt thereof, or any combination thereof.

23. The composition of claim 4, comprising the at least the portion of the plant or the extract thereof which comprises: Boswellia serrata, lemon grass, eucalyptus, bromelain, capsaicin, cat's claw, curcumin, evening primrose, ginger, gingko, green tea, flaxseed, pine bar, rose hips, St. John's wort, Devil's claw, hemp, stinging nettle, thunder god vine, valerian root, any portion thereof, any extract thereof, a tincture thereof, or any combination thereof.

24. The composition of claim 4, comprising the oil which comprises: fish oil, avocado oil, soybean oil, black currant oil, lemon grass oil, eucalyptus oil, frankincense oil, medium-chain triglyceride (MCT) oil, hemp oil, borage oil, or any combination thereof.

25. The composition of claim 4, comprising the fatty acid, which comprises a short chain fatty acid, a medium chain fatty acid, a long chain fatty acid, an ester of any of these, a salt of any of these, or any combination thereof.

26. The composition of claim 4, comprising the fatty acid, which comprises a saturated fatty acid, an unsaturated fatty acid, a monounsaturated fatty acid, a polyunsaturated fatty acid, an ester of any of these, a salt of any of these, or any combination thereof.

27. The composition of claim 26, comprising the saturated fatty acid, the unsaturated fatty acid, the monounsaturated fatty acid, the polyunsaturated fatty acid, or any combination thereof which comprises arachidonic acid, linoleic acid, palmitic acid, oleic acid, adrenic acid, docosapentaenoic acid, mead acid, an ester of any of these, a salt of any of these, or any combination thereof.

28. The composition of claim 4, comprising the fatty acid, which comprises an omega-3 fatty acid, an omega-6 fatty acid, an omega-9 fatty acid, an ester of any of these, a salt of any of these, or any combination thereof.

29. The composition of claim 28, comprising the omega-3 fatty acid, which comprises eicosapentaenoic acid (EPA), docosahexaenoic acid (DHA), alpha-linolenic acid (ALA), an ester of any of these, a salt of any of these, or any combination thereof.

30. The composition of claim 2, wherein the composition is in the form of the capsule, and wherein the capsule is a hydroxymethylcellulose capsule.

31. The composition of claim 2, wherein the composition is in the form of the candy, and wherein the candy is in the form of: a caramel, a fruit candy drop, or any combination thereof.

32. The composition of claim 2, wherein the composition is in the form of the chocolate, and wherein the chocolate is in the form of: a blueberry chocolate, a cranberry chocolate, a fruit flavored chocolate, a citrus flavored chocolate, a fruit covered chocolate, a chocolate bar, a chocolate caramel, a coffee bean chocolate or any combination thereof.

33. The composition of claim 2, wherein the composition is in the form of the balm, and wherein the balm further comprises shea butter, cocoa butter, bees wax, or any combination thereof.

34. The composition of claim 2, wherein the composition is in the form of the cream, and wherein the cream further comprises hemp cream.

35. The composition of claim 2, wherein the composition is in the form of the gummy, and wherein the gummy further comprises carrageenan.

36. A method comprising administering the composition of any one of claims 1-35 to a subject, wherein the composition is in unit dose form.

37. The method of claim 36, wherein the administering is by oral ingestion, topical application, or inhalation.

38. The method of claim 37, comprising the oral ingestion which comprises oral ingestion of a food, a liquid, a gel, a capsule, or any combination thereof.

39. The method of claim 37, comprising the topical application which comprises topical application of a lotion, a tincture, a balm, a cream, an oil, a gel, a butter, a liquid, a spray, an ointment, a paste, a jelly, or any combination thereof.

40. The method of claim 37, comprising the inhalation which comprises inhalation by a diffuser.

41. The method of claim 36, wherein the administering is performed at least about: 1 time per day, 2 times per day, 3 times per day, 4 times per day, 5 times per day, or 6 times per day.

42. The method of claim 36, wherein the administering is performed for about: 1 day to about 8 days, 1 week to about 5 weeks, 1 month to about 12 months, 1 year to about 3 years, 3 years to about 10 years, 10 years to about 50 years, 25 years to about 100 years, or about 50 years to about 130 years.

43. The method of claim 36, wherein the subject has joint pain, join inflammation, joint immobilization, joint deterioration, or any combination thereof.

44. The method of claim 43, wherein the subject was previously diagnosed with acute arthritis, chronic arthritis, or any combination thereof.

45. The method of claim 44, comprising the acute arthritis which comprises: an infection, gout, calcium pyrophosphate arthritis, bursitis, tendinitis, or any combination thereof.

46. The method of claim 44, comprising the chronic arthritis which comprises: rheumatoid arthritis, psoriatic arthritis, lupus, erythematosus, osteoarthritis, juvenile idiopathic arthritis, fibromyalgia, polymyalgia rheumatica, or any combination thereof.

47. The method of claim 36, wherein the subject is a human.

48. The method of claim 47, wherein the human is from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

49. The method of claim 36, wherein the subject is an animal.

50. The method of claim 49, wherein the animal comprises a pet.

51. The method of claim 50, wherein the pet comprises a dog, a cat, a rodent, a horse, a goat, or a rabbit.

52. The method of claim 49, wherein the animal is from about 1 day to about 10 months old, from about 9 months to about 24 months old, from about 1 year to about 8 years old, from about 5 years to about 25 years old, or from about 20 years to about 50 years old.

53. A method of treating or preventing a disease comprising administering the composition of any one of claims 1-35 to a subject as a pharmaceutical composition in unit dose form.

54. The method of claim 53, wherein the disease comprises acute arthritis, chronic arthritis, or any combination thereof.

55. The method of claim 54, wherein the disease comprises acute arthritis which comprises: an infection, gout, calcium pyrophosphate arthritis, bursitis, tendinitis, or any combination thereof.

56. The method of claim 55, wherein the disease comprises chronic arthritis which comprises: rheumatoid arthritis, psoriatic arthritis, lupus, erythematosus, osteoarthritis, juvenile idiopathic arthritis, fibromyalgia, polymyalgia rheumatica, or any combination thereof.

57. The method of claim 53, wherein prior to treating, the subject has been diagnosed with acute arthritis, chronic arthritis, or any combination thereof.

58. The method of claim 53, further comprising diagnosing the subject as having acute arthritis, chronic arthritis, or any combination thereof.

59. The method of claim 58, wherein the diagnosis comprises a physical examination, a radiological image, a blood test, an antibody test, or any combination thereof.

60. The method of claim 59, comprising the radiological image which comprises: a computed tomography (CT) image, an X-Ray image, a magnetic resonance image (MRI), an ultrasound image, or any combination thereof.

61. The method of claim 53, further comprising administering a second therapy to the subject.

62. The method of claim 61, wherein the second therapy comprises acetaminophen, an opioid, a nonsteroidal anti-inflammatory drug, methotrexate, hydroxychloroquine, prednisone, cortisone, a biological response modifier, a salt thereof, or any combination thereof.

63. The method of claim 62, wherein the second therapy comprises the biological response modifier which comprises: abatacept, adalimumab, adalimumab-atto, anakinra, certolizumab pegol, etanercept, etanercept-szzs, golimumab, infliximab, infliximab-dyyb, rituximab, sarilumab, tocilizumab, a biologically active fragment of any of these, a salt of any of these, or any combination thereof.

64. The method of claim 62, wherein the second therapy comprises the nonsteroidal anti-inflammatory drug which comprises naproxen, ibuprofen, a salt of any of these, or any combination thereof.

65. The method of claim 53, wherein the composition further comprises an excipient, a diluent, a carrier, or any combination thereof.

66. The method of claim 53, wherein the administering is by oral ingestion, topical application, or inhalation.

67. The method of claim 66, comprising the oral ingestion which comprises oral ingestion of a food, a liquid, a gel, a capsule, a tablet, or any combination thereof.

68. The method of claim 66, comprising the topical application which comprises topical application of a lotion, a tincture, a balm, a cream, an oil, a gel, a butter, a liquid, a spray, an ointment, a paste, a jelly, or any combination thereof.

69. The method of claim 66, comprising the inhalation which comprises inhalation by a diffuser, an inhaler, a nebulizer or any combination thereof.

70. The method of claim 53, wherein the administering is performed at least about: 1 time per day, 2 times per day, 3 times per day, 4 times per day, 5 times per day, or 6 times per day.

71. The method of claim 53, wherein the administering is performed for about: 1 day to about 8 days, 1 week to about 5 weeks, 1 month to about 12 months, 1 year to about 3 years, 3 years to about 10 years, 10 years to about 50 years, 25 years to about 100 years, or about 50 years to about 130 years.

72. The method of claim 53, wherein the subject is a subject in need thereof.

73. The method of claim 53, wherein the subject is a human.

74. The method of claim 73, wherein the human is from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

75. The method of claim 53, wherein the subject is an animal.

76. The method of claim 75, wherein the animal comprises a pet.

77. The method of claim 76, comprising the pet which comprises a dog, a cat, a rodent, a horse, a goat, or a rabbit.

78. The method of claim 75, wherein the animal is about 1 day to about 10 months old, from about 9 months to about 24 months old, from about 1 year to about 8 years old, from about 5 years to about 25 years old, or from about 20 years to about 50 years old.

79. A method of making a composition of any one of claim 1-35, comprising formulating the following into a composition: CBD, a flavanone or a salt thereof; and one or more of: a glucosamine, a salt thereof, a glycosaminoglycan, a salt thereof, a vitamin, a derivative thereof, a salt thereof, or methylsulfonylmethane (MSM).

80. A method of making a composition, comprising formulating the following into a composition: CBD, naringenin or a salt thereof, and one or more of eucalyptus oil or lemon grass oil.

81. The method of claim 80, further comprising formulating D-limonene, a salt thereof, vitamin C, a salt thereof, MSM, a hemp cream, or any combination thereof into the composition.

82. The method of claim 80, wherein the CBD is at least partially water soluble.

83. The method of any one of claims 80-82, wherein the composition is formulated as a cream or a lotion.

84. A method of making a composition, comprising formulating the following into a composition: a medium-chain triglyceride (MCT) oil, CBD, and naringenin or a salt thereof.

85. The method of claim 84, further comprising formulating shea butter, cocoa butter, bees wax, lemon grass oil, frankincense oil, eucalyptus oil, D-limonene or a salt thereof, MSM, vitamin C or a salt thereof, or any combination thereof into the composition.

86. The method of claim 84, further comprising formulating a flavor or a fragrance into the composition.

87. The method of claim 84, wherein the CBD is at least partially water soluble.

88. The method of any one of claims 84-87, further comprising formulating the composition as a balm or a tincture.

89. A kit comprising the composition of any one of claims 1-35, and a container.
